SEQUENCE LISTING (86 sequences)

SEQ ID NO: 1 - protein, 447 aa, Artificial Sequence
  Heavy Chain Amino Acid Sequence; Anti-LAG-3 mAb (BMS-986016)
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence
  Light Chain Amino Acid Sequence; Anti-LAG-3 mAb (BMS-986016)
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, Artificial Sequence
  Heavy Chain Variable Region (VH) Amino Acid Sequence; Anti-LAG-3 mAb (BMS-986016)
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 4 - protein, 107 aa, Artificial Sequence
  Light Chain Variable Region (VL) Amino Acid Sequence; Anti-LAG-3 mAb (BMS-986016)
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 5 - protein, 5 aa, Artificial Sequence
  Heavy Chain CDR1 Amino Acid Sequence; Anti-LAG-3 mAb (BMS-986016)
DYYWN

SEQ ID NO: 6 - protein, 16 aa, Artificial Sequence
  Heavy Chain CDR2 Amino Acid Sequence; Anti-LAG-3 mAb (BMS-986016)
EINHRGSTNSNPSLKS

SEQ ID NO: 7 - protein, 12 aa, Artificial Sequence
  Heavy Chain CDR3 Amino Acid Sequence; Anti-LAG-3 mAb (BMS-986016)
GYSDYEYNWFDP

SEQ ID NO: 8 - protein, 11 aa, Artificial Sequence
  Light Chain CDR1 Amino Acid Sequence; Anti-LAG-3 mAb (BMS-986016)
RASQSISSYLA

SEQ ID NO: 9 - protein, 7 aa, Artificial Sequence
  Light Chain CDR2 Amino Acid Sequence; Anti-LAG-3 mAb (BMS-986016)
DASNRAT

SEQ ID NO: 10 - protein, 9 aa, Artificial Sequence
  Light Chain CDR3 Amino Acid Sequence; Anti-LAG-3 mAb (BMS-986016)
QQRSNWPLT

SEQ ID NO: 11 - protein, 440 aa, Artificial Sequence
  Heavy Chain Amino Acid Sequence; Anti-PD-1 mAb (BMS-936558)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 213 aa, Artificial Sequence
  Light Chain Amino Acid Sequence; Anti-PD-1 mAb (BMS-936558)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLLSKADYEKHKVYACEVTHQGLSSPVTKSFNRGEC

SEQ ID NO: 13 - protein, 113 aa, Artificial Sequence [sequence omitted]

SEQ ID NO: 14 - protein, 107 aa, Artificial Sequence [sequence omitted]

SEQ ID NO: 15 - protein, 5 aa, Artificial Sequence
  Heavy Chain CDR1 Amino Acid Sequence; Anti-PD-1 mAb (BMS-936558)
NSGMH

SEQ ID NO: 16 - protein, 17 aa, Artificial Sequence
  Heavy Chain CDR2 Amino Acid Sequence; Anti-PD-1 mAb (BMS-936558)
VIWYDGSKRYYADSVKG

SEQ ID NO: 17 - protein, 4 aa, Artificial Sequence
  Heavy Chain CDR3 Amino Acid Sequence; Anti-PD-1 mAb (BMS-936558)
NDDY

SEQ ID NO: 18 - protein, 11 aa, Artificial Sequence
  Light Chain CDR1 Amino Acid Sequence; Anti-PD-1 mAb (BMS-936558)
RASQSVSSYLA

SEQ ID NO: 19 - protein, 7 aa, Artificial Sequence
  Light Chain CDR2 Amino Acid Sequence; Anti-PD-1 mAb (BMS-936558)
DASNRAT

SEQ ID NO: 20 - protein, 9 aa, Artificial Sequence
  Light Chain CDR3 Amino Acid Sequence; Anti-PD-1 mAb (BMS-936558)
QQSSNWPRT

SEQ ID NO: 21 - protein, 446 aa, Artificial Sequence
  Heavy Chain Amino Acid Sequence; Anti-LAG-3 mAb (BMS-986016)
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 525 aa, Artificial Sequence
  Lymphocyte Activation Gene 3 Protein Amino Acid Sequence
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 23 - protein, 449 aa, artificial sequence
  Heavy Chain Amino Acid Sequence; Anti-LAG-3 mAb (REGN3767)
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, artificial sequence
  Light Chain Amino Acid Sequence; Anti-LAG-3 mAb (REGN3767)
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 123 aa, artificial sequence
  Heavy Chain Variable Region (VH) Amino Acid Sequence; Anti-LAG-3 mAb (REGN3767)
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 26 - protein, 107 aa, artificial sequence
  Light Chain Variable Region (VL) Amino Acid Sequence; Anti-LAG-3 mAb (REGN3767)
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 27 - protein, 8 aa, artificial sequence
  Heavy Chain CDR1 Amino Acid Sequence; Anti-LAG-3 mAb (REGN3767)
GFTFSSYG

SEQ ID NO: 28 - protein, 8 aa, artificial sequence
  Heavy Chain CDR2 Amino Acid Sequence; Anti-LAG-3 mAb (REGN3767)
IWYDGSNK

SEQ ID NO: 29 - protein, 16 aa, artificial sequence
  Heavy Chain CDR3 Amino Acid Sequence; Anti-LAG-3 mAb (REGN3767)
ASVATSGDFDYYGMDV

SEQ ID NO: 30 - protein, 6 aa, artificial sequence
  Light Chain CDR1 Amino Acid Sequence; Anti-LAG-3 mAb (REGN3767)
QRISTY

SEQ ID NO: 31 - protein, 3 aa, artificial sequence
  Light Chain CDR2 Amino Acid Sequence; Anti-LAG-3 mAb (REGN3767)
DAS

SEQ ID NO: 32 - protein, 9 aa, artificial sequence
  Light Chain CDR3 Amino Acid Sequence; Anti-LAG-3 mAb (REGN3767)
QQRSNWPLT

SEQ ID NO: 33 - protein, 444 aa, artificial sequence
  Heavy Chain Amino Acid Sequence; Anti-PD-1 mAb (REGN2810)
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 214 aa, artificial sequence
  Light Chain Amino Acid Sequence; Anti-PD-1 mAb (REGN2810)
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 117 aa, artificial sequence [sequence omitted]

SEQ ID NO: 36 - protein, 107 aa, artificial sequence [sequence omitted]

SEQ ID NO: 37 - protein, 8 aa, artificial sequence
  Heavy Chain CDR1 Amino Acid Sequence; Anti-PD-1 mAb (REGN2810)
GFTFSNFG

SEQ ID NO: 38 - protein, 8 aa, artificial sequence
  Heavy Chain CDR2 Amino Acid Sequence; Anti-PD-1 mAb (REGN2810)
ISGGGRDT

SEQ ID NO: 39 - protein, 10 aa, artificial sequence
  Heavy Chain CDR3 Amino Acid Sequence; Anti-PD-1 mAb (REGN2810)
VKWGNIYFDY

SEQ ID NO: 40 - protein, 6 aa, artificial sequence
  Light Chain CDR1 Amino Acid Sequence; Anti-PD-1 mAb (REGN2810)
LSINTF

SEQ ID NO: 41 - protein, 3 aa, artificial sequence
  Light Chain CDR2 Amino Acid Sequence; Anti-PD-1 mAb (REGN2810)
AAS

SEQ ID NO: 42 - protein, 9 aa, artificial sequence
  Light Chain CDR3 Amino Acid Sequence; Anti-PD-1 mAb (REGN2810)
QQSSNTPFT

SEQ ID NO: 43 - protein, 451 aa, artificial sequence
  Heavy Chain Amino Acid Sequence; Anti-LAG-3 mAb (LAG525)
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 451 aa, artificial sequence
  Heavy Chain Amino Acid Sequence; Anti-LAG-3 mAb (LAG525)
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 214 aa, artificial sequence
  Light Chain Amino Acid Sequence; Anti-LAG-3 mAb (LAG525)
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 214 aa, artificial sequence
  Light Chain Amino Acid Sequence; Anti-LAG-3 mAb (LAG525)
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 125 aa, artificial sequence
  Heavy Chain Variable Region (VH) Amino Acid Sequence; Anti-LAG-3 mAb (LAG525)
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 48 - protein, 125 aa, artificial sequence
  Heavy Chain Variable Region (VH) Amino Acid Sequence; Anti-LAG-3 mAb (LAG525)
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 49 - protein, 107 aa, artificial sequence
  Light Chain Variable Region (VL) Amino Acid Sequence; Anti-LAG-3 mAb (LAG525)
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 50 - protein, 107 aa, artificial sequence
  Light Chain Variable Region (VL) Amino Acid Sequence; Anti-LAG-3 mAb (LAG525)
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 51 - protein, 5 aa, artificial sequence
  Heavy Chain CDR1 Amino Acid Sequence; Anti-LAG-3 mAb (LAG525)
NYGMN

SEQ ID NO: 52 - protein, 17 aa, artificial sequence
  Heavy Chain CDR2 Amino Acid Sequence; Anti-LAG-3 mAb (LAG525)
WINTDTGEPTYADDFKG

SEQ ID NO: 53 - protein, 16 aa, artificial sequence
  Heavy Chain CDR3 Amino Acid Sequence; Anti-LAG-3 mAb (LAG525)
NPPYYYGTNNAEAMDY

SEQ ID NO: 54 - protein, 11 aa, artificial sequence
  Light Chain CDR1 Amino Acid Sequence; Anti-LAG-3 mAb (LAG525)
SSSQDISNYLN

SEQ ID NO: 55 - protein, 7 aa, artificial sequence
  Light Chain CDR2 Amino Acid Sequence; Anti-LAG-3 mAb (LAG525)
YTSTLHL

SEQ ID NO: 56 - protein, 9 aa, artificial sequence
  Light Chain CDR3 Amino Acid Sequence; Anti-LAG-3 mAb (LAG525)
QQYYNLPWT

SEQ ID NO: 57 - protein, 443 aa, artificial sequence
  Heavy Chain Amino Acid Sequence; Anti-PD-1 mAb (PDR001)
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 220 aa, artificial sequence
  Light Chain Amino Acid Sequence; Anti-PD-1 mAb (PDR001)
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 117 aa, artificial sequence
  Heavy Chain Variable Region (VH) Amino Acid Sequence; Anti-PD-1 mAb (PDR001)
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 60 - protein, 113 aa, artificial sequence
  Light Chain Variable Region (VL) Amino Acid Sequence; Anti-PD-1 mAb (PDR001)
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 61 - protein, 5 aa, artificial sequence
  Heavy Chain CDR1 Amino Acid Sequence; Anti-PD-1 mAb (PDR001)
TYWMH

SEQ ID NO: 62 - protein, 17 aa, artificial sequence
  Heavy Chain CDR2 Amino Acid Sequence; Anti-PD-1 mAb (PDR001)
NIYPGTGGSNFDEKFKN

SEQ ID NO: 63 - protein, 8 aa, artificial sequence
  Heavy Chain CDR3 Amino Acid Sequence; Anti-PD-1 mAb (PDR001)
WTTGTGAY

SEQ ID NO: 64 - protein, 17 aa, artificial sequence
  Light Chain CDR1 Amino Acid Sequence; Anti-PD-1 mAb (PDR001)
KSSQSLLDSGNQKNFLT

SEQ ID NO: 65 - protein, 7 aa, artificial sequence
  Light Chain CDR2 Amino Acid Sequence; Anti-PD-1 mAb (PDR001)
WASTRES

SEQ ID NO: 66 - protein, 9 aa, artificial sequence
  Light Chain CDR3 Amino Acid Sequence; Anti-PD-1 mAb (PDR001)
QNDYSYPYT

SEQ ID NO: 67 - protein, 446 aa, artificial sequence
  Heavy Chain Amino Acid Sequence; Anti-LAG-3 mAb (MK4280)
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 218 aa, artificial sequence
  Light Chain Amino Acid Sequence; Anti-LAG-3 mAb (MK4280)
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 119 aa, artificial sequence
  Heavy Chain Variable Region (VH) Amino Acid Sequence; Anti-LAG-3 mAb (MK4280)
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 70 - protein, 111 aa, artificial sequence
  Light Chain Variable Region (VL) Amino Acid Sequence; Anti-LAG-3 Anti-LAG-3 mAb (MK4280)
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 71 - protein, 5 aa, artificial sequence
  Heavy Chain CDR1 Amino Acid Sequence; Anti-LAG-3 mAb (MK4280)
DYNVD

SEQ ID NO: 72 - protein, 17 aa, artificial sequence
  Heavy Chain CDR2 Amino Acid Sequence; Anti-LAG-3 mAb (MK4280)
DINPNDGGTIYAQKFQE

SEQ ID NO: 73 - protein, 10 aa, artificial sequence
  Heavy Chain CDR3 Amino Acid Sequence; Anti-LAG-3 mAb (MK4280)
NYRWFGAMDH

SEQ ID NO: 74 - protein, 15 aa, artificial sequence
  Light Chain CDR1 Amino Acid Sequence; Anti-LAG-3 mAb (MK4280)
KASQSLDYEGDSDMN

SEQ ID NO: 75 - protein, 7 aa, artificial sequence
  Light Chain CDR2 Amino Acid Sequence; Anti-LAG-3 mAb (MK4280)
GASNLES

SEQ ID NO: 76 - protein, 9 aa, artificial sequence
  Light Chain CDR3 Amino Acid Sequence; Anti-LAG-3 mAb (MK4280)
QQSTEDPRT

SEQ ID NO: 77 - protein, 447 aa, artificial sequence
  Heavy Chain Amino Acid Sequence; Anti-PD-1 mAb (MK3475)
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 218 aa, artificial sequence
  Light Chain Amino Acid Sequence; Anti-PD-1 mAb (MK3475)
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 120 aa, artificial sequence [sequence omitted]

SEQ ID NO: 80 - protein, 111 aa, artificial sequence [sequence omitted]

SEQ ID NO: 81 - protein, 5 aa, artificial sequence
  Heavy Chain CDR1 Amino Acid Sequence; Anti-PD-1 mAb (MK3475)
NYYMY

SEQ ID NO: 82 - protein, 17 aa, artificial sequence
  Heavy Chain CDR2 Amino Acid Sequence; Anti-PD-1 mAb (MK4280)
GINPSNGGTNFNEKFKN

SEQ ID NO: 83 - protein, 11 aa, artificial sequence
  Heavy Chain CDR3 Amino Acid Sequence; Anti-PD-1 mAb (MK3475)
RDYRFDMGFDY

SEQ ID NO: 84 - protein, 15 aa, artificial sequence
  Light Chain CDR1 Amino Acid Sequence; Anti-PD-1 mAb (MK3475)
RASKGVSTSGYSYLH

SEQ ID NO: 85 - protein, 7 aa, artificial sequence
  Light Chain CDR2 Amino Acid Sequence; Anti-PD-1 mAb (MK3475)
LASYLES

SEQ ID NO: 86 - protein, 9 aa, artificial sequence
  Light Chain CDR3 Amino Acid Sequence; Anti-PD-1 mAb (MK3475)
QHSRDLPLT

(19) United States
(12) Patent Application Publication
(10) Pub. No.: US 2024/0101666 A1
(43) Pub. Date: Mar. 28, 2024

(54) LAG-3 ANTAGONIST THERAPY FOR LUNG CANCER

(71) Applicant: Bristol-Myers Squibb Company, Princeton, NJ (US)

(72) Inventors: Laurence David TOMS, Princeton, NJ (US); Paul Andrew BASCIANO, Princeton, NJ (US)

(73) Assignee: Bristol-Myers Squibb Company, Princeton, NJ (US)

(21) Appl. No.: 18/033,200

(22) PCT Filed: Oct. 22, 2021
(86) PCT No.: PCT/US2021/056241

§ 371 Date: Apr. 21, 2023

Related U.S. Application Data
(60) Provisional application No. 63/110,210, filed on Nov. 5, 2020.
(60) Provisional application No. 63/104,744, filed on Oct. 23, 2020.

(51) Int. Cl. C07K 16/28; A61K 31/282; A61K 31/337; A61K 31/519; A61K 33/243; A61P 35/00

(52) U.S. Cl. CPC C07K 16/2803; A61K 31/282; A61K 31/337; A61K 31/519; A61K 33/243; A61P 35/00; C07K 16/2818; A61K 2039/507

(57) ABSTRACT

The disclosure provides a method of treating a human subject afflicted with lung cancer with a lymphocyte activation gene-3 (LAG-3) antagonist. In some aspects, the method comprises combination of the LAG-3 antagonist with an additional therapeutic agent (e.g., a programmed death-1 pathway inhibitor) and/or anti-cancer therapy (e.g., chemotherapy such as a platinum doublet chemotherapy).

### CROSS REFERENCE TO RELATED APPLICATIONS

This PCT application claims the priority benefit of U.S. Provisional Application Nos. 63/104,744, filed Oct. 23, 2020, and 63/110,210, filed Nov. 5, 2020, which are incorporated herein by reference in their entireties.

### REFERENCE TO SEQUENCE LISTING SUBMITTED ELECTRONICALLY VIA EFS-WEB

The content of the electronically submitted sequence listing (Name: 3338_240PC02_Seqlisting_ST25.txt; Size: 94,766 Bytes; and Date of Creation: Oct. 21, 2021) is herein incorporated by reference in its entirety.

### FIELD OF THE INVENTION

The present disclosure provides methods of treating human subjects afflicted with lung cancer comprising a lymphocyte activation gene-3 (LAG-3) antagonist.

### BACKGROUND OF THE INVENTION

Lung cancer, and particularly non-small cell lung cancer (NSCLC) remains the leading cause of cancer-related mortality worldwide, accounting for approximately 18% of all cancer deaths (Jenal A, et al., CA Cancer J. Clin. 2011; 61:69-90).

Until recently, the treatment of patients with advanced NSCLC whose tumors did not have a targetable genetic alteration was cytotoxic chemotherapy alone. In spite of treatment, patients with metastatic NSCLC treated with platinum doublet chemotherapy had a median survival of approximately 10 months and a 5-year survival rate of less than 5%. The introduction of immune checkpoint inhibitors targeting the PD-1 signaling pathway in the treatment of patients with NSCLC has had a significant effect on patient survival. The anti-PD-1 antibody pembrolizumab combined with chemotherapy in the front-line setting has demonstrated an improvement in overall survival in NSCLC patients as compared to chemotherapy alone (Gandhi L, et al., N. Engl. J. Med. 2018; 378:2078-2092; Paz-Ares L, et al., N. Engl. J. Med. 2018; 379:2040-2051). More recently, the anti-PD-1 antibody nivolumab plus the anti-CTLA-4 antibody ipilimumab, as well as nivolumab plus ipilimumab in combination with chemotherapy, also showed benefit over chemotherapy in this setting (Peters S, et al., Annals of Oncology 2019; 30 (suppl_5):v851-v934; Reck M, J. Clin. Oncol. 2020; (suppl):abstr. 9501). However, despite these advances, the median survival of first line patients with metastatic NSCLC is approximately 22 months in the non-squamous and 15.9 months in the squamous population (Paz-Ares L, et al.; Gadgeel S, et al., J. Clin. Oncol. 2020; 38(14):1505-1517).

There is a need for improved methods for treating human subjects afflicted with lung cancer.

### SUMMARY OF THE INVENTION

The present disclosure is directed to method of treating a human subject afflicted with lung cancer, the method comprising administering to the subject a lymphocyte activation gene-3 (LAG-3) antagonist and a platinum doublet chemotherapy (PDCT).

In some aspects, the method is a first line therapy.

In some aspects, the method is a second line therapy.

In some aspects, the method is a third line therapy.

In some aspects, the subject has progressed on a prior therapy.

In some aspects, the lung cancer is recurrent following multi-modal therapy for locally advanced lung cancer.

In some aspects, the subject has not received a prior systemic therapy for cancer, the subject has not received a prior systemic therapy for lung cancer, or the subject has not received a prior systemic therapy for advanced or metastatic lung cancer.

In some aspects, the subject is naïve to prior immuno-oncology therapy, the subject is naïve to prior immuno-oncology therapy for lung cancer, or the lung cancer is naïve to prior immuno-oncology therapy.

In some aspects, the lung cancer is unresectable, advanced, recurrent, and/or metastatic.

In some aspects, the subject is afflicted with a Stage IV lung cancer.

In some aspects, the lung cancer is small cell lung cancer.

In some aspects, the lung cancer is non-small cell lung cancer (NSCLC). In some aspects, the NSCLC has a squamous or non-squamous histology.

In some aspects, one or more immune cells in tumor tissue from the subject express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, the immune cells are tumor-infiltrating lymphocytes. In some aspects, the tumor-infiltrating lymphocytes are CD8⁺ cells.

In some aspects, one or more tumor cells in tumor tissue from the subject express PD-L1. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1.

In some aspects, the LAG-3 antagonist is an anti-LAG-3 antibody.

In some aspects, the anti-LAG-3 antibody is a full-length antibody.

In some aspects, the anti-LAG-3 antibody is a monoclonal, human, humanized, chimeric, or multispecific antibody. In some aspects, the multispecific antibody is a dual-affinity re-targeting antibody (DART), a DVD-Ig, or bispecific antibody.

In some aspects, the anti-LAG-3 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG-3 antibody is BMS-986016 (relatlimab), IMP731 (H5L7BW), MK4280 (28G-10, favezelimab), REGN3767 (fianlimab), GSK2831781, humanized BAP050, IMP-701 (LAG525, ieramilimab), aLAG3(0414), aLAG3(0416), Sym022, TSR-033, TSR-075, XmAb841 (XmAb22841), MGD013 (tebotelimab), B1754111, FS118, P 13B02-30, AVA-017, 25F7, AGEN1746, R07247669, INCAGNO2385, IBI-110, EMB-02, IBI-323, LBL-007, ABL501, or comprises an antigen binding portion thereof.

In some aspects, the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4.

In some aspects, the anti-LAG-3 antibody comprises: (a) a heavy chain variable region CDR1 comprising the sequence set forth in SEQ ID NO:5; (b) a heavy chain variable region CDR2 comprising the sequence set forth in SEQ ID NO: 6; (c) a heavy chain variable region CDR3 comprising the sequence set forth in SEQ ID NO:7; (d) a light chain variable region CDR1 comprising the sequence set forth in SEQ ID NO:8; (e) a light chain variable region CDR2 comprising the sequence set forth in SEQ ID NO:9; and (f) a light chain variable region CDR3 comprising the sequence set forth in SEQ ID NO:10.

In some aspects, the anti-LAG-3 antibody comprises heavy and light chain variable regions comprising the sequences set forth in SEQ ID NOs:3 and 4, respectively.

In some aspects, the anti-LAG-3 antibody comprises heavy and light chains comprising the sequences set forth in SEQ ID NOs:1 and 2, respectively.

In some aspects, the anti-LAG-3 antibody comprises heavy and light chains comprising the sequences set forth in SEQ ID NOs:21 and 2, respectively.

In some aspects, the LAG-3 antagonist is a soluble LAG-3 polypeptide. In some aspects, the soluble LAG-3 polypeptide is a fusion polypeptide. In some aspects, the soluble LAG-3 polypeptide comprises a ligand binding fragment of the LAG-3 extracellular domain. In some aspects, the ligand binding fragment of the LAG-3 extracellular domain comprises an amino acid sequence with at least about 90%, at least about 95%, at least about 98%, at least about 99%, or about 100% sequence identity to SEQ ID NO:22. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide is IMP321 (eftilagimod alpha).

In some aspects, the LAG-3 antagonist is formulated for intravenous administration.

In some aspects, the LAG-3 antagonist is administered at a flat dose.

In some aspects, the LAG-3 antagonist is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the LAG-3 antagonist is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the LAG-3 antagonist is administered at a weight-based dose.

In some aspects, the LAG-3 antagonist is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the LAG-3 antagonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the PDCT comprises a platinum agent in combination with a nucleoside analog, an antimetabolite, a taxane, a vinca alkaloid, or a topisomerase inhibitor. In some aspects, the platinum agent is cisplatin, carboplatin, oxaliplatin, satraplatin, picoplatin, nedaplatin, triplatin, lipoplatin, or phenanthriplatin. In some aspects, the platinum agent is cisplatin. In some aspects, the platinum agent is carboplatin. In some aspects, the nucleoside analog is cytarabine, gemcitabine, lamivudine, entecavir, or telbivudine. In some aspects, the nucleoside analog is gemcitabine. In some aspects, the antimetabolite is capecitabine, cladribine, clofarabine, cytarabine, floxuridine, fludarabine, fluorouracil, mercaptopurine, methotrexate, pemetrexed, pentostatin, pralatrexate, or thioguanine. In some aspects, the antimetabolite is pemetrexed. In some aspects, the taxane is paclitaxel, albumin-bound paclitaxel, docetaxel, or cabazitaxel. In some aspects, the vinca alkaloid is vinblastine, vincristine, vinorelbine, vindesine, vincaminol, vineridine, or vinburnine. In some aspects, the vinca alkaloid is vinorelbine or vinblastine. In some aspects, the topoisomerase inhibitor is etoposide, mitoxantrone, doxorubicin, irinotecan, topotecan, or camptothecin. In some aspects, the topoisomerase inhibitor is etoposide. In some aspects, the topoisomerase inhibitor is irinotecan.

In some aspects, the PDCT comprises cisplatin or carboplatin in combination with gemcitabine, pemetrexed, paclitaxel, albumin-bound paclitaxel, docetaxel, vinorelbine, vinblastine, etoposide, or irinotecan.

In some aspects, the PDCT comprises cisplatin or carboplatin in combination with paclitaxel or albumin-bound paclitaxel.

In some aspects, the PDCT comprises cisplatin or carboplatin in combination with pemetrexed.

In some aspects, the method further comprises administering to the subject an additional therapeutic agent. In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor comprises afatinib, erlotinib, dacomitinib, gefitinib, osimertinib, alectinib, brigatinib, ceritinib, crizotinib, lorlatinib, entrectinib, dabrafenib, trametinib, vemurafenib, larotrectinib, or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-IA), a epidermal growth factor (EGF), EGF receptor (EGFR), or any combination thereof.

In some aspects, the anti-angiogenesis agent comprises bevacizumab, ramucirumab, aflibercept, tanibirumab, olaratumab, nesvacumab, AMG780, MEDI3617, vanucizumab, rilotumumab, ficlatuzumab, TAK-701, onartuzumab, emibetuzumab, or any combination thereof.

In some aspects, the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

In some aspects, the checkpoint inhibitor comprises a PD-1 pathway inhibitor.

In some aspects, the PD-1 pathway inhibitor is an anti-PD-1 antibody and/or an anti-PD-L1 antibody.

In some aspects, the PD-1 pathway inhibitor is an anti-PD-1 antibody.

In some aspects, the anti-PD-1 antibody is a full-length antibody.

In some aspects, the anti-PD-1 antibody is a monoclonal, human, humanized, chimeric, or multispecific antibody. In some aspects, the multispecific antibody is a DART, a DVD-Ig, or bispecific antibody.

In some aspects, the anti-PD-1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-1 antibody is nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

In some aspects, the anti-PD-1 antibody comprises CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

In some aspects, the anti-PD-1 antibody comprises: (a) a heavy chain variable region CDR1 comprising the sequence set forth in SEQ ID NO:15; (b) a heavy chain variable region CDR2 comprising the sequence set forth in SEQ ID NO:16; (c) a heavy chain variable region CDR3 comprising the sequence set forth in SEQ ID NO:17; (d) a light chain variable region CDR1 comprising the sequence set forth in SEQ ID NO: 18; (e) a light chain variable region CDR2 comprising the sequence set forth in SEQ ID NO: 19; and (f) a light chain variable region CDR3 comprising the sequence set forth in SEQ ID NO:20.

In some aspects, the anti-PD-1 antibody comprises heavy and light chain variable regions comprising the sequences set forth in SEQ ID NOs:13 and 14, respectively.

In some aspects, the anti-PD-1 antibody comprises heavy and light chains comprising the sequences as set forth in SEQ ID NOs:11 and 12, respectively.

In some aspects, the PD-1 pathway inhibitor is a soluble PD-L2 polypeptide. In some aspects, the soluble PD-L2 polypeptide is a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224.

In some aspects, the PD-1 pathway inhibitor is an anti-PD-L1 antibody.

In some aspects, the anti-PD-L1 antibody is a full-length antibody.

In some aspects, the anti-PD-L1 antibody is a monoclonal, human, humanized, chimeric, or multispecific antibody. In some aspects, the multispecific antibody is a DART, a DVD-Ig, or bispecific antibody.

In some aspects, the anti-PD-L1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-L1 antibody is BMS-936559, atezolizumab, durvalumab, avelumab, STI-1014, CX-072, KN035, LY3300054, BGB-A333, ICO 36, FAZ053, CK-301, or comprises an antigen binding portion thereof.

In some aspects, the PD-1 pathway inhibitor is BMS-986189.

In some aspects, the checkpoint inhibitor comprises a CTLA-4 inhibitor.

In some aspects, the CTLA-4 inhibitor is an anti-CTLA-4 antibody.

In some aspects, the anti-CTLA-4 antibody is a full-length antibody.

In some aspects, the anti-CTLA-4 antibody is a monoclonal, human, humanized, chimeric, or multispecific antibody. In some aspects, the multispecific antibody is a DART, a DVD-Ig, or bispecific antibody.

In some aspects, the anti-CTLA-4 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-CTLA-4 antibody is ipilimumab, tremelimumab, MK-1308, AGEN-1884, or comprises an antigen binding portion thereof.

In some aspects, the checkpoint inhibitor is formulated for intravenous administration.

In some aspects, the LAG-3 antagonist and the checkpoint inhibitor are formulated separately. In some aspects, each checkpoint inhibitor is formulated separately when the checkpoint inhibitor comprises more than one checkpoint inhibitor. In some aspects, the checkpoint inhibitor is administered before the LAG-3 antagonist. In some aspects, the LAG-3 antagonist is administered before the checkpoint inhibitor.

In some aspects, the LAG-3 antagonist and the checkpoint inhibitor are formulated together. In some aspects, two or more checkpoint inhibitors are formulated together when the checkpoint inhibitor comprises more than one checkpoint inhibitor.

In some aspects, the LAG-3 antagonist and the checkpoint inhibitor are administered concurrently.

In some aspects, the checkpoint inhibitor is administered at a flat dose.

In some aspects, the checkpoint inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the checkpoint inhibitor is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the checkpoint inhibitor is administered as a weight-based dose.

In some aspects, the checkpoint inhibitor is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the checkpoint inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

The present disclosure is directed to a method of treating a human subject afflicted with lung cancer, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, and (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

The present disclosure is directed to a method of treating a human subject afflicted with lung cancer, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

In some aspects, the method is a first line therapy.

In some aspects, the method is a second line therapy.

In some aspects, the method is a third line therapy.

In some aspects, the subject has progressed on a prior therapy.

In some aspects, the lung cancer is unresectable, advanced, recurrent, and/or metastatic.

In some aspects, the subject is afflicted with a Stage IV lung cancer.

In some aspects, the lung cancer is small cell lung cancer.

In some aspects, the lung cancer is non-small cell lung cancer (NSCLC). In some aspects, the NSCLC has a squamous histology. In some aspects, the NSCLC has a non-squamous histology.

In some aspects, the method further comprises administering a PDCT. In some aspects, the PDCT comprises a platinum agent in combination with a nucleoside analog, an antimetabolite, a taxane, a vinca alkaloid, or a topisomerase inhibitor. In some aspects, the platinum agent is cisplatin, carboplatin, oxaliplatin, satraplatin, picoplatin, nedaplatin, triplatin, lipoplatin, or phenanthriplatin. In some aspects, the platinum agent is cisplatin. In some aspects, the platinum agent is carboplatin. In some aspects, the nucleoside analog is cytarabine, gemcitabine, lamivudine, entecavir, or telbivudine. In some aspects, the nucleoside analog is gemcitabine. In some aspects, the antimetabolite is capecitabine, cladribine, clofarabine, cytarabine, floxuridine, fludarabine, fluorouracil, mercaptopurine, methotrexate, pemetrexed, pentostatin, pralatrexate, or thioguanine. In some aspects, the antimetabolite is pemetrexed. In some aspects, the taxane is paclitaxel, albumin-bound paclitaxel, docetaxel, or cabazitaxel. In some aspects, the vinca alkaloid is vinblastine, vincristine, vinorelbine, vindesine, vincaminol, vineridine, or vinburnine. In some aspects, the vinca alkaloid is vinorelbine or vinblastine. In some aspects, the topoisomerase inhibitor is etoposide, mitoxantrone, doxorubicin, irinotecan, topotecan, or camptothecin. In some aspects, the topoisomerase inhibitor is etoposide. In some aspects, the topoisomerase inhibitor is irinotecan. In some aspects, the PDCT comprises cisplatin or carboplatin in combination with gemcitabine, pemetrexed, paclitaxel, albumin-bound paclitaxel, docetaxel, vinorelbine, vinblastine, etoposide, or irinotecan. In some aspects, the PDCT comprises cisplatin or carboplatin in combination with paclitaxel or albumin-bound paclitaxel. In some aspects, the PDCT comprises cisplatin or carboplatin in combination with pemetrexed.

The present disclosure is directed to a method of treating a human subject afflicted with Stage IV or recurrent NSCLC that has a squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 200 mg/m² of paclitaxel, wherein the method is a first line therapy.

The present disclosure is directed to a method of treating a human subject afflicted with Stage IV or recurrent NSCLC that has a squamous histology, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 200 mg/m² of paclitaxel, wherein the method is a first line therapy.

The present disclosure is directed to a method of treating a human subject afflicted with Stage IV or recurrent NSCLC that has a squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 100 mg/m² of albumin-bound paclitaxel, wherein the method is a first line therapy.

The present disclosure is directed to a method of treating a human subject afflicted with Stage IV or recurrent NSCLC that has a squamous histology, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 100 mg/m² of albumin-bound paclitaxel, wherein the method is a first line therapy.

The present disclosure is directed to a method of treating a human subject afflicted with Stage IV or recurrent NSCLC that has a non-squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 5 mg/mL·min or about 6 mg/mL·min, and (ii) a dose of about 500 mg/m² of pemetrexed, wherein the method is a first line therapy.

The present disclosure is directed to a method of treating a human subject afflicted with Stage IV or recurrent NSCLC that has a non-squamous histology, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 5 mg/mL·min or about 6 mg/mL·min, and (ii) a dose of about 500 mg/m² of pemetrexed, wherein the method is a first line therapy.

The present disclosure is directed to a method of treating a human subject afflicted with Stage IV or recurrent NSCLC that has a non-squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of about 75 mg/m² of cisplatin, and (ii) a dose of about 500 mg/m² of pemetrexed, wherein the method is a first line therapy.

The present disclosure is directed to a method of treating a human subject afflicted with Stage IV or recurrent NSCLC that has a non-squamous histology, the method comprising administering to the subject: (a) a dose of about 720 mg of an anti-LAG-3 antibody comprising CDR1, CDR2, and CDR3 domains of the heavy chain variable region having the sequence set forth in SEQ ID NO:3, and CDR1, CDR2, and CDR3 domains of the light chain variable region having the sequence set forth in SEQ ID NO:4, (b) a dose of about 360 mg of an anti-PD-1 antibody comprising CDR1, CDR2, and CDR3 domains of the heavy chain variable region having the sequence set forth in SEQ ID NO:13, and CDR1, CDR2, and CDR3 domains of the light chain variable region having the sequence set forth in SEQ ID NO:14, (c) a PDCT comprising: (i) a dose of about 75 mg/m² of cisplatin, and (ii) a dose of about 500 mg/m² of pemetrexed, wherein the method is a first line therapy.

In some aspects, the lung cancer is recurrent following multi-modal therapy for locally advanced lung cancer.

In some aspects, the subject has not received a prior systemic therapy for cancer, the subject has not received a prior systemic therapy for lung cancer, or the subject has not received a prior systemic therapy for advanced or metastatic lung cancer.

In some aspects, the subject is naïve to prior immuno-oncology therapy, the subject is naïve to prior immuno-oncology therapy for lung cancer, or the lung cancer is naïve to prior immuno-oncology therapy.

In some aspects, one or more immune cells in tumor tissue from the subject express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, the immune cells are tumor-infiltrating lymphocytes. In some aspects, the tumor-infiltrating lymphocytes are CD8⁺ cells.

In some aspects, one or more tumor cells in tumor tissue from the subject express PD-L1. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1.

In some aspects, (a) the anti-LAG-3 antibody comprises a heavy chain variable region CDR1, CDR2, and CDR3 comprising the sequence set forth in SEQ ID NO:5, SEQ ID NO:6, and SEQ ID NO:7, respectively, and a light chain variable region CDR1, CDR2, and CDR3 comprising the sequence set forth in SEQ ID NO:8, SEQ ID NO:9, and SEQ ID NO:10, respectively, and (b) the anti-PD-1 antibody comprises a heavy chain variable region CDR1, CDR2, and CDR3 comprising the sequence set forth in SEQ ID NO:15, SEQ ID NO:16, and SEQ ID NO:17, respectively, and a light chain variable region CDR1, CDR2, and CDR3 comprising the sequence set forth in SEQ ID NO:18, SEQ ID NO:19, and SEQ ID NO:20, respectively.

In some aspects, the anti-LAG-3 antibody comprises heavy and light chain variable regions comprising the sequences set forth in SEQ ID NOs:3 and 4, respectively, and the anti-PD-1 antibody comprises heavy and light chain variable regions comprising the sequences set forth in SEQ ID NOs:13 and 14, respectively.

In some aspects, the anti-LAG-3 antibody and/or the anti-PD-1 antibody is a full-length antibody.

In some aspects, the anti-LAG-3 antibody and/or anti-PD-1 antibody is a monoclonal, human, humanized, chimeric, or multispecific antibody. In some aspects, the multispecific antibody is a DART, a DVD-Ig, or bispecific antibody.

In some aspects, the anti-LAG-3 antibody and/or anti-PD-1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG-3 antibody comprises heavy and light chains comprising the sequences set forth in SEQ ID NOs:1 and 2, respectively, and the anti-PD-1 antibody comprises heavy and light chains comprising the sequences as set forth in SEQ ID NOs:11 and 12, respectively.

In some aspects, the anti-LAG-3 antibody comprises heavy and light chains comprising the sequences set forth in SEQ ID NOs:21 and 2, respectively, and the anti-PD-1 antibody comprises heavy and light chains comprising the sequences as set forth in SEQ ID NOs:11 and 12, respectively.

In some aspects, the method further comprises administering to the subject an additional therapeutic agent. In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor is afatinib, erlotinib, dacomitinib, gefitinib, osimertinib, alectinib, brigatinib, ceritinib, crizotinib, lorlatinib, entrectinib, dabrafenib, trametinib, vemurafenib, larotrectinib, or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGF receptor (EGFR), or any combination thereof.

In some aspects, the anti-angiogenesis agent comprises bevacizumab, ramucirumab, aflibercept, tanibirumab, olaratumab, nesvacumab, AMG780, MEDI3617, vanucizumab, rilotumumab, ficlatuzumab, TAK-701, onartuzumab, emibetuzumab, or any combination thereof.

In some aspects, the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-0) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

In some aspects, the PD-1 pathway inhibitor is an anti-PD-L1 antibody.

In some aspects, the anti-PD-L1 antibody is a full-length antibody.

In some aspects, the anti-PD-L1 antibody is a monoclonal, human, humanized, chimeric, or multispecific antibody. In some aspects, the multispecific antibody is a DART, a DVD-Ig, or bispecific antibody.

In some aspects, the anti-PD-L1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-L1 antibody is BMS-936559, atezolizumab, durvalumab, avelumab, STI-1014, CX-072, KN035, LY3300054, BGB-A333, ICO 36, FAZ053, CK-301, or comprises an antigen binding portion thereof.

In some aspects, the PD-1 pathway inhibitor is BMS-986189.

In some aspects, the checkpoint inhibitor comprises a CTLA-4 inhibitor.

In some aspects, the CTLA-4 inhibitor is an anti-CTLA-4 antibody.

In some aspects, the anti-CTLA-4 antibody is a full-length antibody.

In some aspects, the anti-CTLA-4 antibody is a monoclonal, human, humanized, chimeric, or multispecific antibody. In some aspects, the multispecific antibody is a DART, a DVD-Ig, or bispecific antibody.

In some aspects, the anti-CTLA-4 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-CTLA-4 antibody is ipilimumab, tremelimumab, MK-1308, AGEN-1884, or comprises an antigen binding portion thereof.

In some aspects, the anti-LAG-3 antibody and the anti-PD-1 antibody are formulated for intravenous administration.

In some aspects, the checkpoint inhibitor is formulated for intravenous administration.

In some aspects, the anti-LAG-3 antibody and the anti-PD-1 antibody are formulated separately.

In some aspects, the anti-LAG-3 antibody and the anti-PD-1 antibody are formulated together.

In some aspects, the anti-PD-1 antibody is administered before the anti-LAG-3 antibody.

In some aspects, the anti-LAG-3 antibody is administered before the anti-PD-1 antibody.

In some aspects, the LAG-3 antibody and the anti-PD-1 antibody are administered concurrently.

In some aspects, the anti-LAG-3 antibody and the anti-PD-1 antibody are administered about once every three weeks. In some aspects, the anti-LAG-3 antibody and the anti-PD-1 antibody are administered on Day 1 of every three-week cycle. In some aspects, the anti-LAG-3 antibody and the anti-PD-1 antibody are administered intravenously from a single intravenous bag for about 30 minutes.

In some aspects, the PDCT is administered every three weeks. In some aspects, the PDCT is administered for up to about 4 three-week cycles.

The present disclosure is directed to a pharmaceutical composition comprising (a) 360 mg of an anti-LAG-3 antibody and (b) 360 mg of an anti-PD-1 antibody.

The present disclosure is directed to a pharmaceutical composition comprising (a) 720 mg of an anti-LAG-3 antibody and (b) 360 mg of an anti-PD-1 antibody

In some aspects, (a) the anti-LAG-3 antibody comprises CDR1, CDR2, and CDR3 domains of the heavy chain variable region having the sequence set forth in SEQ ID NO:3, and CDR1, CDR2, and CDR3 domains of the light chain variable region having the sequence set forth in SEQ ID NO:4, and (b) the anti-PD-1 antibody comprises CDR1, CDR2, and CDR3 domains of the heavy chain variable region having the sequence set forth in SEQ ID NO:13, and CDR1, CDR2, and CDR3 domains of the light chain variable region having the sequence set forth in SEQ ID NO:14.

In some aspects, (a) the anti-LAG-3 antibody comprises a heavy chain variable region CDR1, CDR2, and CDR3 comprising the sequence set forth in SEQ ID NO:5, SEQ ID NO:6, and SEQ ID NO:7, respectively, and a light chain variable region CDR1, CDR2, and CDR3 comprising the sequence set forth in SEQ ID NO:8, SEQ ID NO:9, and SEQ ID NO:10, respectively, and (b) the anti-PD-1 antibody comprises a heavy chain variable region CDR1, CDR2, and CDR3 comprising the sequence set forth in SEQ ID NO:15, SEQ ID NO:16, and SEQ ID NO:17, respectively, and a light chain variable region CDR1, CDR2, and CDR3 comprising the sequence set forth in SEQ ID NO:18, SEQ ID NO:19, and SEQ ID NO:20, respectively.

In some aspects, the anti-LAG-3 antibody comprises heavy and light chain variable regions comprising the sequences set forth in SEQ ID NOs:3 and 4, respectively, and the anti-PD-1 antibody comprises heavy and light chain variable regions comprising the sequences set forth in SEQ ID NOs:13 and 14, respectively.

In some aspects, the anti-LAG-3 antibody and/or the anti-PD-1 antibody is a full-length antibody.

In some aspects, the anti-LAG-3 antibody and/or anti-PD-1 antibody is a monoclonal, human, humanized, chimeric, or multispecific antibody. In some aspects, the multispecific antibody is a DART, a DVD-Ig, or bispecific antibody.

In some aspects, the anti-LAG-3 antibody and/or anti-PD-1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG-3 antibody comprises heavy and light chains comprising the sequences set forth in SEQ ID NOs:1 and 2, respectively, and the anti-PD-1 antibody comprises heavy and light chains comprising the sequences as set forth in SEQ ID NOs:11 and 12, respectively.

In some aspects, the anti-LAG-3 antibody comprises heavy and light chains comprising the sequences set forth in SEQ ID NOs:21 and 2, respectively, and the anti-PD-1 antibody comprises heavy and light chains comprising the sequences as set forth in SEQ ID NOs:11 and 12, respectively.

The present disclosure is directed to a kit for treating a human subject afflicted with lung cancer, comprising: (a) 360 mg of an anti-LAG-3 antibody; (b) 360 mg of an anti-PD-1 antibody; and (c) instructions for using the anti-LAG-3 antibody and the anti-PD-1 antibody in a method for treating a human subject afflicted with lung cancer.

The present disclosure is directed to a kit for treating a human subject afflicted with lung cancer, comprising: (a) 720 mg of an anti-LAG-3 antibody; (b) 360 mg of an anti-PD-1 antibody; and (c) instructions for using the anti-LAG-3 antibody and the anti-PD-1 antibody in a method for treating a human subject afflicted with lung cancer.

### DETAILED DESCRIPTION OF THE INVENTION

The present disclosure provides a method of treating a human subject afflicted with lung cancer (e.g., non-small cell lung cancer (NSCLC)), the method comprising administering to the subject a LAG-3 antagonist (e.g., an anti-LAG-3 antibody). Some aspects of the present disclosure are directed to a method of treating a human subject afflicted with lung cancer, wherein the method is a first, second, or third line therapy. Some aspects of the present disclosure are directed to a method of treating a human subject afflicted with Stage IV or recurrent lung cancer. The present disclosure is also directed to methods of treating a human subject afflicted with lung cancer comprising an anti-cancer therapy and/or a therapeutic agent in combination with the LAG-3 antagonist, such as a chemotherapy (e.g., platinum doublet chemotherapy) and/or a PD-1 pathway inhibitor (e.g., an anti-PD-1 antibody).

### I. Terms

In order that the present disclosure can be more readily understood, certain terms are first defined. As used in this application, except as otherwise expressly provided herein, each of the following terms shall have the meaning set forth below. Additional definitions are set forth throughout the application. It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

The headings provided herein are not limitations of the various aspects of the disclosure, which can be had by reference to the specification as a whole. Accordingly, the terms defined immediately below are more fully defined by reference to the specification in its entirety.

An “antagonist” shall include, without limitation, any molecule capable of blocking, reducing, or otherwise limiting an interaction or activity of a target molecule (e.g., LAG-3). In some aspects, the antagonist is an antibody. In other aspects, the antagonist comprises a small molecule. The terms “antagonist” and “inhibitor” are used interchangeably herein.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region (abbreviated herein as C_{H}). The heavy chain constant region comprises three constant domains, C_{H1}, C_{H2} and C_{H3}. Each light chain comprises a light chain variable region (abbreviated herein as V_{L}) and a light chain constant region (abbreviated herein as C_{L}). The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain can have the C-terminal lysine or not. Unless specified otherwise herein, the amino acids in the variable regions are numbered using the Kabat numbering system and those in the constant regions are numbered using the EU system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; single chain antibodies; monospecific antibodies; bispecific antibodies; and multi-specific antibodies. A nonhuman antibody can be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, that retains the ability to bind specifically to the antigen bound by the whole immunoglobulin. Examples of an “antigen-binding portion” or “antigen-binding fragment” include: (1) a Fab fragment (fragment from papain cleavage) or a similar monovalent fragment consisting of the V_{L}, V_{H}, L_{C} and C_{H1} domains; (2) a F(ab′)₂ fragment (fragment from pepsin cleavage) or a similar bivalent fragment comprising two Fab fragments linked by a disulfide bridge at the hinge region; (3) a Fd fragment consisting of the V_{H} and CH1 domains; (4) a Fv fragment consisting of the V_{L} and V_{H} domains of a single arm; (5) a single domain antibody (dAb) fragment (Ward et al., (1989) Nature 341:544-46), which consists of a V_{H} domain; (6) a bi-single domain antibody which consists of two V_{H} domains linked by a hinge (dual-affinity re-targeting antibodies (DARTs)); or (7) a dual variable domain immunoglobulin. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883).

An “isolated antibody” refers to an antibody that is substantially free of other antibodies having different antigenic specificities (e.g., an isolated antibody that binds specifically to LAG-3 is substantially free of antibodies that do not bind specifically to LAG-3). An isolated antibody that binds specifically to LAG-3 can, however, have cross-reactivity to other antigens, such as LAG-3 molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-LAG-3 antibody binds specifically to LAG-3.

“LAG-3” refers to Lymphocyte Activation Gene-3. The term “LAG-3” includes variants, isoforms, homologs, orthologs and paralogs. For example, antibodies specific for a human LAG-3 protein can, in certain cases, cross-react with a LAG-3 protein from a species other than human. In other aspects, the antibodies specific for a human LAG-3 protein can be completely specific for the human LAG-3 protein and not exhibit species or other types of cross-reactivity, or can cross-react with LAG-3 from certain other species, but not all other species (e.g., cross-react with monkey LAG-3 but not mouse LAG-3). The term “human LAG-3” refers to human sequence LAG-3, such as the complete amino acid sequence of human LAG-3 having GenBank Accession No. NP_002277. The term “mouse LAG-3” refers to mouse sequence LAG-3, such as the complete amino acid sequence of mouse LAG-3 having GenBank Accession No. NP_032505. LAG-3 is also known in the art as, for example, CD223. The human LAG-3 sequence can differ from human LAG-3 of GenBank Accession No. NP_002277 by having, e.g., conserved mutations or mutations in non-conserved regions, and the LAG-3 has substantially the same biological function as the human LAG-3 of GenBank Accession No. NP_002277. For example, a biological function of human LAG-3 is having an epitope in the extracellular domain of LAG-3 that is specifically bound by an antibody of the instant disclosure or a biological function of human LAG-3 is binding to MHC Class II molecules.

A particular human LAG-3 sequence will generally be at least about 90% identical in amino acid sequence to human LAG-3 of GenBank Accession No. NP_002277 and contains amino acid residues that identify the amino acid sequence as being human when compared to LAG-3 amino acid sequences of other species (e.g., murine). In certain cases, a human LAG-3 can be at least about 95%, or even at least about 96%, at least about 97%, at least about 98%, at least about 99%, or about 100% identical in amino acid sequence to LAG-3 of GenBank Accession No. NP_002277. In certain aspects, a human LAG-3 sequence will display no more than 10 amino acid differences from the LAG-3 sequence of GenBank Accession No. NP_002277. In certain aspects, the human LAG-3 can display no more than 5, or even no more than 4, 3, 2, or 1 amino acid difference from the LAG-3 sequence of GenBank Accession No. NP_002277.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. “PD-1” and “PD-1 receptor” are used interchangeably herein.

“Cytotoxic T-Lymphocyte Antigen-4 (CTLA-4)” refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

“Programmed Death Ligand-2 (PD-L2)” as used herein includes human PD-L2 (hPD-L2), variants, isoforms, and species homologs of hPD-L2, and analogs having at least one common epitope with hPD-L2. The complete hPD-L2 sequence can be found under GenBank Accession No. Q9BQ51.

A “patient” as used herein includes any patient who is afflicted with a lung cancer (e.g., NSCLC). The terms “subject” and “patient” are used interchangeably herein.

“Administering” refers to the physical introduction of a therapeutic agent to a subject (e.g., a composition or formulation comprising the therapeutic agent), using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, in some aspects, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease. Response Evaluation Criteria In Solid Tumors (RECIST) is a measure for treatment efficacy and are established rules that define when tumors respond, stabilize, or progress during treatment. RECIST 1.1 is the current guideline to solid tumor measurement and definitions for objective assessment of change in tumor size for use in adult and pediatric cancer clinical trials.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment can refer to alleviation of at least one symptom of a solid tumor. Such effective treatment can, e.g., reduce patient pain, reduce the size and/or number of lesions, can reduce or prevent metastasis of a tumor, and/or can slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-LAG-3 antibody alone or the amount of anti-LAG-3 antibody and the amount an additional therapeutic agent (e.g., anti-PD-1 antibody), in combination, clinically proven to affect a significant decrease in cancer or slowing of progression of cancer, such as an advanced solid tumor.

As used herein, the terms “fixed dose,” “flat dose,” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., an amount in g or mg).

The use of the term “fixed dose combination” with regard to a composition of the invention means that two or more different inhibitors as described herein (e.g., an anti-LAG-3 antibody and an anti-PD-1 antibody) in a single composition are present in the composition in particular (fixed) ratios with each other. In some aspects, the fixed dose is based on the weight (e.g., mg) of the inhibitors. In certain aspects, the fixed dose is based on the concentration (e.g., mg/ml) of the inhibitors. In some aspects, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first inhibitor to mg second inhibitor. For example, the 2:1 ratio of a first inhibitor and a second inhibitor can mean that a vial can contain about 720 mg of the first inhibitor and 360 mg of the second inhibitor or about 12 mg/ml of the first inhibitor and 6 mg/ml of the second inhibitor.

The term “weight based dose” as referred to herein means that a dose that is administered to a patient is calculated based on the weight of the patient.

“Dosing interval,” as used herein, means the amount of time that elapses between multiple doses of a formulation disclosed herein being administered to a subject. Dosing interval can thus be indicated as ranges.

The term “dosing frequency” as used herein refers to the frequency of administering doses of a formulation disclosed herein in a given time. Dosing frequency can be indicated as the number of doses per a given time, e.g., once a week or once in two weeks, etc.

The terms “about once a week,” “once about every week,” “once about every two weeks,” or any other similar dosing interval terms as used herein means approximate number, and “about once a week” or “once about every week” can include every seven days±two days, i.e., every five days to every nine days. The dosing frequency of “once a week” thus can be every five days, every six days, every seven days, every eight days, or every nine days. “Once about every three weeks” can include every 21 days±3 days, i.e., every 25 days to every 31 days. Similar approximations apply, for example, to once about every two weeks, once about every four weeks, once about every five weeks, once about every six weeks, once about every seven weeks, once about every eight weeks, once about every nine weeks, once about every ten weeks, once about every eleven weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity.

The term “tumor” as used herein refers to any mass of tissue that results from excessive cell growth or proliferation, either benign (non-cancerous) or malignant (cancerous), including pre-cancerous lesions.

The term “biological sample” as used herein refers to biological material isolated from a subject. The biological sample can contain any biological material suitable for analysis, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. The biological sample can be a test tissue sample (e.g., a tissue sample comprising tumor cells and tumor-infiltrating inflammatory cells). In one aspect, the sample is a tumor tissue biopsy, e.g., a formalin-fixed, paraffin-embedded (FFPE) tumor tissue or a fresh-frozen tumor tissue or the like. In another aspect, the biological sample is a liquid biopsy that, in some aspects, comprises one or more of blood, serum, plasma, circulating tumor cells, exoRNA, ctDNA, and cfDNA.

By way of example, an “anti-cancer agent” promotes cancer regression in a subject. In preferred aspects, a therapeutically effective amount of the agent promotes cancer regression to the point of eliminating the cancer. “Promoting cancer regression” means that administering an effective amount of the anti-cancer agent, alone or in combination with another agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the agent to promote cancer regression in the patient. Physiological safety refers to the level of toxicity, or other adverse physiological effects at the cellular, organ and/or organism level (adverse effects) resulting from administration of the agent.

By way of example for the treatment of tumors, a therapeutically effective amount of an anti-cancer agent can inhibit cell growth or tumor growth by at least about 20%, at least about 40%, at least about 60%, or at least about 80% relative to untreated subjects. In other aspects of the disclosure, tumor regression can be observed and continue for a period of at least about 20 days, more preferably at least about 40 days, or at least about 60 days. Notwithstanding these measurements of therapeutic effectiveness, evaluation of immunotherapeutic drugs must also make allowance for immune-related response patterns.

As used herein, an “immuno-oncology” therapy or an “I-O” or “IO” therapy refers to a therapy that comprises utilizing an immune response to target and treat a tumor in a subject. As such, as used herein, an I-O therapy is a type of anti-cancer therapy. In some aspects, an I-O therapy comprises administering an antibody to a subject. In some aspects, an I-O therapy comprises administering to a subject an immune cell, e.g., a T cell, e.g., a modified T cell, e.g., a T cell modified to express a chimeric antigen receptor or a particular T cell receptor. In some aspects, the I-O therapy comprises administering a therapeutic vaccine to a subject. In some aspects, the I-O therapy comprises administering a cytokine or a chemokine to a subject. In some aspects, the I-O therapy comprises administering an interleukin to a subject. In some aspects, the I-O therapy comprises administering an interferon to a subject. In some aspects, the I-O therapy comprises administering a colony stimulating factor to a subject.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

A “tumor-infiltrating inflammatory cell” or “tumor-associated inflammatory cell” is any type of cell that typically participates in an inflammatory response in a subject and which infiltrates tumor tissue. Such cells include tumor-infiltrating lymphocytes (TILs), macrophages, monocytes, eosinophils, histiocytes and dendritic cells.

The term “LAG-3 positive” or “LAG-3 expression positive,” relating to LAG-3 expression, refers to tumor tissue (e.g., a test tissue sample) that is scored as expressing LAG-3 based on the proportion (i.e., percentage) of immune cells (e.g., tumor-infiltrating lymphocytes such as CD8+ T cells) expressing LAG-3 (e.g., greater than or equal to 1% expression).

“LAG-3 negative” or “LAG-3 expression negative,” refers to tumor tissue (e.g., a test tissue sample) that is not scored as expressing LAG-3 (e.g., less than 1% LAG-3 expression).

The term “PD-1 positive” or “PD-1 expression positive,” relating to PD-1 expression, refers to tumor tissue (e.g., a test tissue sample) that is scored as expressing PD-1 based on the proportion (i.e., percentage) of immune cells (e.g., tumor-infiltrating lymphocytes such as CD8+ T cells) expressing PD-1 (e.g., greater than or equal to 1% expression).

“PD-1 negative” or “PD-1 expression negative,” refers to tumor tissue (e.g., a test tissue sample) that is not scored as expressing PD-1 (e.g., less than 1% PD-1 expression).

The term “PD-L1 positive” or “PD-L1 expression positive,” relating to cell surface PD-L1 expression, refers to tumor tissue (e.g., a test tissue sample) that is scored as expressing PD-L1 based on the proportion (i.e., percentage) of tumor cells expressing PD-L1 (e.g., greater than or equal to 1% expression).

The term “PD-L1 negative” or “PD-L1 expression negative” refers to tumor tissue (e.g., a test tissue sample) that is not scored as expressing PD-L1 (e.g., less than 1% expression).

Various aspects of the invention are described in further detail in the following subsections.

### II. Methods of the Disclosure

Provided herein are methods of treating a human subject afflicted with lung cancer, the methods comprising administering to the subject a LAG-3 antagonist (e.g., an anti-LAG-3 antibody) alone or in combination with one or more additional therapeutic agents (e.g., a PD-1 pathway inhibitor such as an anti-PD-1 antibody) and/or therapies (e.g., a chemotherapy such as a platinum doublet chemotherapy).

In some aspects, the method is a first line (1L) therapy.

In some aspects, the method is a second line (2L) therapy.

In some aspects, the method is a third line (3L) therapy.

In some aspects, the subject has progressed on a prior therapy (e.g., a standard of care therapy). Standard of care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard of care treatments for a wide variety of cancers. See NCCN GUIDELINES®, 2020, https://www.nccn.org/professionals/physician_gls/default.aspx, last accessed Oct. 23, 2020.

In some aspects, the lung cancer is recurrent following multi-modal therapy for locally advanced lung cancer.

In some aspects, the subject has not received a prior systemic therapy for cancer, the subject has not received a prior systemic therapy for lung cancer, or the subject has not received a prior systemic therapy for advanced or metastatic lung cancer.

In some aspects, the subject is naïve to prior immuno-oncology (I-O) therapy. In some aspects, the subject has never received I-O therapy, has received I-O therapy for a cancer other than lung cancer, or has received I-O therapy for a previous lung cancer but not a current lung cancer. In some aspects, the subject is naïve to prior I-O therapy, the subject is naïve to prior I-O therapy for lung cancer, or the lung cancer is naïve to prior I-O therapy. In some aspects, the prior I-O therapy is an antibody. In some aspects, the antibody binds to a checkpoint inhibitor. In some aspects, the prior I-O therapy is an anti-PD-1 antibody and/or the combination of an anti-PD-1 antibody and an anti-CTLA-4 antibody.

In some aspects, a method of the disclosure increases duration of progression-free survival (PFS), objective response rate (ORR), overall survival (OS), or any combination thereof as compared to a standard of care therapy and/or a prior therapy such as disclosed herein.

In some aspects, a method of the disclosure reduces the size of a tumor, inhibits growth of a tumor, eliminates a tumor from the subject, prevents relapse of lung cancer, induces remission of lung cancer, provides a complete response or partial response, or any combination thereof.

In some aspects, the methods of the disclosure comprise administering to the subject a LAG-3 antagonist based on the subject's performance status and/or cancer stage. Performance status and/or cancer stage can be indicated by any one or more systems in the art.

In some aspects, the lung cancer is unresectable, advanced, recurrent, and/or metastatic.

In some aspects, performance status is indicated by Eastern Cooperative Oncology Group performance status (ECOG PS), which utilizes standardized criteria for measuring how disease impacts a patient's daily living abilities. Example definitions for ECOG PS include: “0” for a patient who is fully active and able to carry on all pre-disease performance without restriction; “1” for a patient who is restricted in physically strenuous activity but ambulatory and able to carry out work of a light or sedentary nature; “2” for a patient who is ambulatory and capable of all self-care, up and about more than 50% of waking hours, but unable to carry out any work activities; “3” for a patient who is capable of only limited self-care and is confined to a bed or chair more than 50% of waking hours; and “4” for a patient who is completely disabled, cannot carry on any self-care, and is totally confined to bed or chair.

In some aspects the subject has an ECOG PS of 0, 1, 2, 3, or 4. In some aspects, the subject has an ECOG PS of ≤3. In some aspects, the subject has an ECOG PS of ≤2. In some aspects, the subject has an ECOG PS of ≤1.

In some aspects, lung cancer is staged based on a tumor/node/metastasis (TNM) staging system such as the American Joint Committee on Cancer (AJCC) classification.

There are at least seven stages used for lung cancer: occult (hidden) stage, Stage 0 (carcinoma in situ), Stage I, Stage II, Stage IIIA, Stage IIIB, and Stage IV. In the occult stage, the cancer cannot be seen by imaging or bronchoscopy. In Stage 0, cancer cells are found in the lining of the airways.

In some aspects the subject is afflicted with a Stage 0 lung cancer.

In some aspects, the subject is afflicted with a Stage I lung cancer. Stage I lung cancer is divided in Stage IA and IB. In Stage IA, the tumor is in the lung only and is 3 centimeters or smaller. In Stage IB, the cancer has not spread to the lymph nodes and one or more of the following is true: 1) the tumor is larger than 3 centimeters but not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus.

In some aspects, the subject is afflicted with a Stage II lung cancer. Stage II is divided into Stage IIA and IIB. In Stage IIA, the cancer has either spread to the lymph nodes or not. If the cancer has spread to the lymph nodes, then the cancer can only have spread to the lymph nodes on the same side of the chest as the tumor, the lymph nodes with cancer are within the lung or near the bronchus, and one or more of the following is true: 1) the tumor is not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIA if the cancer has not spread to the lymph nodes and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. In stage IIB, the cancer has either spread to the lymph nodes or not. If the cancer has spread to the lymph nodes, then the cancer can only have spread to the lymph nodes on the same side of the chest as the tumor, the lymph nodes with cancer are within the lung or near the bronchus, and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIB if the cancer has not spread to the lymph nodes and one or more of the following is true: 1) the tumor is larger than 7 centimeters; 2) the cancer has spread to the main bronchus (and is at least 2 centimeters below where the trachea joins the bronchus), the chest wall, the diaphragm, or the nerve that controls the diaphragm; 3) cancer has spread to the membrane around the heart or lining the chest wall; 4) the whole lung has collapsed or developed pneumonitis (inflammation of the lung); or 5) there are one or more separate tumors in the same lobe of the lung.

In some aspects, the subject is afflicted with a Stage III lung cancer. Stage IIIA is divided into 3 sections. These 3 sections are based on 1) the size of the tumor; 2) where the tumor is found and 3) which (if any) lymph nodes have cancer. In the first type of Stage IIIA, the cancer has spread to the lymph nodes on the same side of the chest as the tumor, and the lymph nodes with the cancer are near the sternum or where the bronchus enters the lung. Additionally: 1) the tumor may be any size; 2) part of the lung (where the trachea joins the bronchus) or the whole lung may have collapsed or developed pneumonitis (inflammation of the lung); 3) there may be one or more separate tumors in the same lobe of the lung; and 4) cancer can have spread to any of the following: a) main bronchus, but not the area where the trachea joins the bronchus, b) chest well, c) diaphragm and the nerve that controls it, d) membrane around the lung or lining the chest wall, e) membrane around the heart. In the second type of Stage IIIA, the cancer has spread to the lymph nodes on the same side of the chest as the tumor, and the lymph nodes with the cancer are within the lung or near the bronchus. Additionally: 1) the tumor may be any size; 2) the whole lung may have collapsed or developed pneumonitis (inflammation of the lung); 3) there may be one or more separate tumors in the any of the lobes of the lung with cancer; and 4) cancer can have spread to any of the following: a) main bronchus, but not the area where the trachea joins the bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the third type of Stage IIIA, the cancer has not spread to the lymph nodes, the tumor may be any size, and cancer has spread to any one of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi). Stage IIIB is divided into 2 sections depending on 1) the size of the tumor, 2) where the tumor is found, and 3) which lymph nodes have cancer. In the first type of Stage IIIB, the cancer has spread to the lymph nodes on the opposite side of the chest as the tumor. Additionally, 1) the tumor may be any size; 2) part of the lung (where the trachea joins the bronchus) or the whole lung may have collapsed or developed pneumonitis (inflammation of the lung); 3) there may be one or more separate tumors in any of the lobs of the lung with cancer; and 4) cancer may have spread to any of the following: a) main bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the second type of Stage IIIB, the cancer has spread to lymph nodes on the same side of the chest as the tumor. The lymph nodes with cancer are near the sternum (chest bone) or where the bronchus enters the lung. Additionally, 1) the tumor may be any size; 2) there may be separate tumors in different lobes of the same lung; and 3) cancer has spread to any of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi).

In some aspects, the subject is afflicted with a Stage IV lung cancer. In Stage IV, the tumor may be any size and the cancer may have spread to the lymph nodes. One or more of the following is true in Stage IV: 1) there are one or more tumors in both lungs; 2) cancer is found in the fluid around the lungs or heart; and 3) cancer has spread to other parts of the body, such as the brain, liver, adrenal glands, kidneys or bone.

In some aspects, the lung cancer is small cell lung cancer (SCLC). In some aspects, staging of SCLC is by TNM staging. In some aspects, rather than TNM staging, SCLC is staged as either limited stage or extensive stage. Limited stage SCLC is confined to one lung and/or the local lymph nodes. Extensive stage SCLC is found in both lungs and/or distant sites in the body.

In some aspects, the lung cancer is non-small cell lung cancer (NSCLC). NSCLC includes NSCLC with a histology that is “not otherwise specified” (NOS), NSCLC with a squamous histology (SQ), and NSCLC with a non-squamous histology (NSQ, including adenocarcinoma, large cell, and undifferentiated carcinoma). In some aspects, the NSCLC has a squamous histology. In some aspects, the NSCLC has a non-squamous histology.

Surgery (i.e., surgical resection), radiation therapy (RT), and chemotherapy are three modalities commonly used to treat NSCLC patients. As a class, NSCLCs are relatively insensitive to chemotherapy and RT, compared to small cell carcinoma. In general, for patients with Stage I or II disease, surgical resection has provided the best chance for cure, with chemotherapy often used both pre-operatively and post-operatively. RT can also be used as adjuvant therapy for patients with resectable NSCLC, the primary local treatment, or as palliative therapy for patients with incurable NSCLC. Patients with advanced or metastatic disease (e.g., Stage IV NSCLC) who have a good performance status (PS) can benefit from chemotherapy.

Specific targeted therapies have also been developed for the treatment of advanced or metastatic NSCLC in subjects with sensitizing mutations in genes for epidermal growth factor receptor (EGFR), anaplastic lymphoma kinase (ALK), ROS-1, neurotrophin receptor tyrosine kinase (NTRK), and B-rapidly accelerated fibrosarcoma proto-oncogene (BRAF, e.g., the BRAF V600E mutation).

In some aspects, the subject has an EGFR, ALK, NTRK, ROS-1, or BRAF mutation sensitive to targeted inhibitor therapy.

In some aspects, the subject has no EGFR, ALK, NTRK, ROS-1, or BRAF mutation sensitive to targeted inhibitor therapy.

In some aspects, one or more immune cells in tumor tissue from the subject express LAG-3 (i.e., tumor tissue from the patient is LAG-3 positive) and/or one or more tumor cells in tumor tissue from the subject express PD-L1 (i.e., tumor tissue from the patient is PD-L1 positive). In some aspects, one or more immune cells in tumor tissue from the subject express LAG-3. In some aspects, at least about 1%, at least about 2%, at least about 3%, at least about 4%, at least about 5%, at least about 7%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, greater than about 1% of the immune cells express LAG-3. In some aspects, at least about 5% of the immune cells express LAG-3. In some aspects, the immune cells are tumor-infiltrating lymphocytes. In some aspects, the tumor-infiltrating lymphocytes are CD8⁺ cells. In some aspects, one or more tumor cells in tumor tissue from the subject express PD-L1. In some aspects, at least about 1%, at least about 2%, at least about 3%, at least about 4%, at least about 5%, at least about 7%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1. In some aspects, at least about 1% of the tumor cells express PD-L1. In some aspects, greater than about 1% of the tumor cells express PD-L1. In some aspects, at least about 5% of the tumor cells express PD-L1. In some aspects, any of the values of “at least about X %” is “≥X %”).

In some aspects, one or more immune cells in tumor tissue from the patient does not express LAG-3 (i.e., tumor tissue from the patient is LAG-3 negative). In some aspects, the tumor tissue is LAG-3 negative when less than about 1% of the immune cells express LAG-3.

In some aspects, one or more immune cells in tumor tissue from the patient does not express PD-1 (i.e., tumor tissue from the patient is PD-1 negative). In some aspects, the tumor tissue is PD-1 negative when less than about 1% of the immune cells express PD-1.

In some aspects, one or more tumor cells in tumor tissue from the patient does not express PD-L1 (i.e., tumor tissue from the patient is PD-L1 negative). In some aspects, the tumor tissue is PD-L1 negative when less than about 1% of the tumor cells express PD-L1.

In some aspects, LAG-3, PD-1, and/or PD-L1 expression in the subject's tumor tissue is determined from a test tissue sample. In some aspects, a test tissue sample includes, but is not limited to, any clinically relevant tissue sample, such as a tumor biopsy, a core biopsy, an incisional biopsy, an excisional biopsy, a surgical specimen, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are from multiple time points, for example, before treatment, during treatment, and/or after treatment. In some aspects, test tissue samples are from different locations in the subject, for example, from a primary tumor and from a metastasis.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., tumor) sample. In some aspects, the test tissue sample is a cell isolated from a fluid. In some aspects, the test tissue sample comprises circulating tumor cells (CTCs). In some aspects, the test tissue sample comprises tumor-infiltrating lymphocytes (TILs). In some aspects, the test tissue sample comprises tumor cells and tumor-infiltrating lymphocytes (TILs). In some aspects, the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In some aspects, LAG-3, PD-1, and/or PD-L1 expression is assessed by performing an assay to detect the presence of LAG-3, PD-1, and/or PD-L1 RNA, respectively. In some aspects, the presence of LAG-3, PD-1, and/or PD-L1 RNA is detected by RT-PCR, in situ hybridization or RNase protection.

In some aspects, LAG-3, PD-1, and/or PD-L1 expression is assessed by performing an assay to detect the presence of LAG-3, PD-1, and/or PD-L1 polypeptide, respectively. In some aspects, the presence of LAG-3, PD-1, and/or PD-L1 polypeptide is detected by immunohistochemistry (IHC), enzyme-linked immunosorbent assay (ELISA), in vivo imaging, or flow cytometry.

### II.A. LAG-3 Antagonists

A LAG-3 antagonist for use in the methods of the disclosure includes, but is not limited to, LAG-3 binding agents and soluble LAG-3 polypeptides. LAG-3 binding agents include antibodies that specifically bind to LAG-3 (i.e., an “anti-LAG-3 antibody”). The term “LAG-3 antagonist” as used herein is interchangeable with the term “LAG-3 inhibitor.”

In some aspects, the LAG-3 antagonist is an anti-LAG-3 antibody.

Antibodies that bind to LAG-3 have been disclosed, for example, in Int'l Publ. No. WO/2015/042246 and U.S. Publ. Nos. 2014/0093511 and 2011/0150892, each of which is incorporated by reference herein in its entirety.

An exemplary LAG-3 antibody useful in the present disclosure is 25F7 (described in U.S. Publ. No. 2011/0150892). An additional exemplary LAG-3 antibody useful in the present disclosure is BMS-986016 (relatlimab). In some aspects, an anti-LAG-3 antibody useful in the present disclosure cross-competes with 25F7 or BMS-986016. In some aspects, an anti-LAG-3 antibody useful in the present disclosure binds to the same epitope as 25F7 or BMS-986016. In some aspects, an anti-LAG-3 antibody comprises six CDRs of 25F7 or BMS-986016.

Other art-recognized anti-LAG-3 antibodies that can be used in the methods of the disclosure include IMP731 (H5L7BW) described in US 2011/007023, MK-4280 (28G-10, favezelimab) described in WO2016028672 and U.S. Publication No. 2020/0055938, REGN3767 (fianlimab) described in Burova E, et al., J. Immunother. Cancer (2016); 4(Supp. 1):P195 and U.S. Pat. No. 10,358,495, humanized BAP050 described in WO2017/019894, GSK2831781, IMP-701 (LAG525; ieramilimab) described in U.S. Pat. No. 10,711,060 and U.S. Publ. No. 2020/0172617, aLAG3(0414), aLAG3(0416), Sym022, TSR-033, TSR-075, XmAb841 (previously XmAb22841), MGD013 (tebotelimab), BI754111, FS118, P 13B02-30, AVA-017, AGEN1746, R07247669, INCAGNO2385, IBI-110, EMB-02, IBI-323, LBL-007, and ABL501. These and other anti-LAG-3 antibodies useful in the claimed invention can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

Anti-LAG-3 antibodies that can be used in the methods of the disclosure also include isolated antibodies that bind specifically to human LAG-3 and cross-compete for binding to human LAG-3 with any anti-LAG-3 antibody disclosed herein, e.g., relatlimab. In some aspects, the anti-LAG-3 antibody binds the same epitope as any of the anti-LAG-3 antibodies described herein, e.g., relatlimab.

In some aspects, the antibodies that cross-compete for binding to human LAG-3 with, or bind to the same epitope region as, any anti-LAG-3 antibody disclosed herein, e.g., relatlimab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

The ability of antibodies to cross-compete for binding to an antigen indicates that the antibodies bind to the same epitope region of the antigen and sterically hinder the binding of other cross-competing antibodies to that particular epitope region. These cross-competing antibodies are expected to have functional properties very similar those of the reference antibody, e.g., relatlimab, by virtue of their binding to the same epitope region. Cross-competing antibodies can be readily identified based on their ability to cross-compete in standard binding assays such as Biacore analysis, ELISA assays or flow cytometry (see, e.g., WO 2013/173223).

Anti-LAG-3 antibodies that can be used in the methods of the disclosure also include antigen-binding portions of any of the above full-length antibodies. It has been amply demonstrated that the antigen-binding function of an antibody can be performed by fragments of a full-length antibody.

In some aspects, the anti-LAG-3 antibody is a full-length antibody.

In some aspects, the anti-LAG-3 antibody is a monoclonal, human, humanized, chimeric, or multispecific antibody. In some aspects, the multispecific antibody is a dual-affinity re-targeting antibody (DART), a DVD-Ig, or bispecific antibody.

In some aspects, the anti-LAG-3 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG-3 antibody is BMS-986016 (relatlimab), IMP731 (H5L7BW), MK4280 (28G-10, favezelimab), REGN3767 (fianlimab), GSK2831781, humanized BAP050, IMP-701 (LAG525, ieramilimab), aLAG3(0414), aLAG3(0416), Sym022, TSR-033, TSR-075, XmAb841 (XmAb22841), MGD013 (tebotelimab), B1754111, FS118, P 13B02-30, AVA-017, 25F7, AGEN1746, R07247669, INCAGNO2385, IBI-110, EMB-02, IBI-323, LBL-007, ABL501, or comprises an antigen binding portion thereof.

In some aspects, the anti-LAG-3 antibody is relatlimab.

In some aspects, the methods of the disclosure comprise an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4.

In some aspects, the methods of the disclosure comprise an anti-LAG-3 antibody comprising: (a) a heavy chain variable region CDR1 comprising the sequence set forth in SEQ ID NO:5; (b) a heavy chain variable region CDR2 comprising the sequence set forth in SEQ ID NO: 6; (c) a heavy chain variable region CDR3 comprising the sequence set forth in SEQ ID NO:7; (d) a light chain variable region CDR1 comprising the sequence set forth in SEQ ID NO:8; (e) a light chain variable region CDR2 comprising the sequence set forth in SEQ ID NO:9; and (f) a light chain variable region CDR3 comprising the sequence set forth in SEQ ID NO:10.

In some aspects, the methods of the disclosure comprise an anti-LAG-3 antibody comprising heavy and light chain variable regions comprising the sequences set forth in SEQ ID NOs:3 and 4, respectively.

In some aspects, the methods of the disclosure comprise an anti-LAG-3 antibody comprising heavy and light chains comprising the sequences set forth in SEQ ID NOs:1 and 2, respectively.

In some aspects, the methods of the disclosure comprise an anti-LAG-3 antibody comprising heavy and light chains comprising the sequences set forth in SEQ ID NOs:21 and 2, respectively.

In some aspects, the anti-LAG-3 antibody is MGD013 (tebotelimab), which is a bispecific PD-1×LAG-3 DART. In some aspects, tebotelimab is administered intravenously at a dose of about 300 mg or about 600 mg once about every 2 or 3 weeks. In some aspects, tebotelimab is administered intravenously at a dose of about 300 mg once about every 2 weeks. In some aspects, tebotelimab is administered intravenously at a dose of about 600 mg once about every 3 weeks.

In some aspects, the anti-LAG-3 antibody is REGN3767 (fianlimab). In some aspects, fianlimab is administered intravenously at a dose of about 1 mg/kg, about 3 mg/kg, about 10 mg/kg, or about 20 mg/kg once about every 3 weeks. In some aspects, fianlimab is administered intravenously at a dose of about 1600 mg once about every 3 weeks. In some aspects, the methods of the disclosure comprise an anti-LAG-3 antibody comprising CDR1, CDR2 and CDR3 domains of the heavy chain variable region having the sequence set forth in SEQ ID NO:25, and CDR1, CDR2 and CDR3 domains of the light chain variable region having the sequence set forth in SEQ ID NO:26.

In some aspects, the methods of the disclosure comprise an anti-LAG-3 antibody comprising: (a) a heavy chain variable region CDR1 comprising the sequence set forth in SEQ ID NO:27; (b) a heavy chain variable region CDR2 comprising the sequence set forth in SEQ ID NO:28; (c) a heavy chain variable region CDR3 comprising the sequence set forth in SEQ ID NO:29; (d) a light chain variable region CDR1 comprising the sequence set forth in SEQ ID NO:30; (e) a light chain variable region CDR2 comprising the sequence set forth in SEQ ID NO:31; and (f) a light chain variable region CDR3 comprising the sequence set forth in SEQ ID NO:32.

In some aspects, the methods of the disclosure comprise an anti-LAG-3 antibody comprising heavy and light chain variable regions comprising the sequences set forth in SEQ ID NOs:25 and 26, respectively.

In some aspects, the methods of the disclosure comprise an anti-LAG-3 antibody comprising heavy and light chains comprising the sequences as set forth in SEQ ID NOs:23 and 24, respectively.

In some aspects, the anti-LAG-3 antibody is LAG525 (ieramilimab). In some aspects, ieramilimab is administered intravenously at a dose of about 300 mg, about 400 mg, about 500 mg, about 600 mg, about 700 mg, about 800 mg, about 900 mg, about 1000 mg, about 1100 mg, about 1200 mg, or about 1300 mg once about every 2, 3, or 4 weeks.

In some aspects, the methods of the disclosure comprise an anti-LAG-3 antibody comprising CDR1, CDR2 and CDR3 domains of the heavy chain variable region having the sequence set forth in SEQ ID NO:47, and CDR1, CDR2 and CDR3 domains of the light chain variable region having the sequence set forth in SEQ ID NO:49.

In some aspects, the methods of the disclosure comprise an anti-LAG-3 antibody comprising CDR1, CDR2 and CDR3 domains of the heavy chain variable region having the sequence set forth in SEQ ID NO:48, and CDR1, CDR2 and CDR3 domains of the light chain variable region having the sequence set forth in SEQ ID NO:50.

In some aspects, the methods of the disclosure comprise an anti-LAG-3 antibody comprising: (a) a heavy chain variable region CDR1 comprising the sequence set forth in SEQ ID NO:51; (b) a heavy chain variable region CDR2 comprising the sequence set forth in SEQ ID NO:52; (c) a heavy chain variable region CDR3 comprising the sequence set forth in SEQ ID NO:53; (d) a light chain variable region CDR1 comprising the sequence set forth in SEQ ID NO:54; (e) a light chain variable region CDR2 comprising the sequence set forth in SEQ ID NO:55; and (f) a light chain variable region CDR3 comprising the sequence set forth in SEQ ID NO:56.

In some aspects, the methods of the disclosure comprise an anti-LAG-3 antibody comprising heavy and light chain variable regions comprising the sequences set forth in SEQ ID NOs:47 and 49, respectively.

In some aspects, the methods of the disclosure comprise an anti-LAG-3 antibody comprising heavy and light chain variable regions comprising the sequences set forth in SEQ ID NOs:48 and 50, respectively.

In some aspects, the methods of the disclosure comprise an anti-LAG-3 antibody comprising heavy and light chains comprising the sequences as set forth in SEQ ID NOs:43 and 45, respectively.

In some aspects, the methods of the disclosure comprise an anti-LAG-3 antibody comprising heavy and light chains comprising the sequences as set forth in SEQ ID NOs:44 and 46, respectively.

In some aspects, the anti-LAG-3 antibody is MK4280 (favezelimab). In some aspects, favezelimabis administered intravenously at a dose of about 7 mg, about 21 mg, about 70 mg, about 210 mg, about 700 mg, or about 800 mg once about every 3 weeks or once about every 6 weeks. In some aspects, favezelimab is administered intravenously at a dose of about 200 mg once about every 3 weeks. In some aspects, favezelimab is administered intravenously at a dose of about 800 mg once about every 6 weeks. In some aspects, favezelimab is administered intravenously at a dose of about 800 mg on Day 1, then once about every 3 weeks. In some aspects, favezelimab is administered for up to 35 cycles. In some aspects, favezelimab is administered intravenously at a dose of about 800 mg for about 30 minutes on Day 1 of a three-week cycle for up to 35 cycles.

In some aspects, the methods of the disclosure comprise an anti-LAG-3 antibody comprising CDR1, CDR2 and CDR3 domains of the heavy chain variable region having the sequence set forth in SEQ ID NO:69, and CDR1, CDR2 and CDR3 domains of the light chain variable region having the sequence set forth in SEQ ID NO:70.

In some aspects, the methods of the disclosure comprise an anti-LAG-3 antibody comprising: (a) a heavy chain variable region CDR1 comprising the sequence set forth in SEQ ID NO:71; (b) a heavy chain variable region CDR2 comprising the sequence set forth in SEQ ID NO:72; (c) a heavy chain variable region CDR3 comprising the sequence set forth in SEQ ID NO:73; (d) a light chain variable region CDR1 comprising the sequence set forth in SEQ ID NO:74; (e) a light chain variable region CDR2 comprising the sequence set forth in SEQ ID NO:75; and (f) a light chain variable region CDR3 comprising the sequence set forth in SEQ ID NO:76.

In some aspects, the methods of the disclosure comprise an anti-LAG-3 antibody comprising heavy and light chain variable regions comprising the sequences set forth in SEQ ID NOs:69 and 70, respectively.

In some aspects, the methods of the disclosure comprise an anti-LAG-3 antibody comprising heavy and light chains comprising the sequences as set forth in SEQ ID NOs:67 and 68, respectively.

In some aspects, the LAG-3 antagonist is a soluble LAG-3 polypeptide. In some aspects, the soluble LAG-3 polypeptide is a fusion polypeptide, e.g., a fusion protein comprising the extracellular portion of LAG-3. In some aspects, the soluble LAG-3 polypeptide is a LAG-3-Fc fusion polypeptide capable of binding to MHC Class II. In some aspects, the soluble LAG-3 polypeptide comprises a ligand binding fragment of the LAG-3 extracellular domain. In some aspects, the ligand binding fragment of the LAG-3 extracellular domain comprises an amino acid sequence with at least about 90%, at least about 95%, at least about 98%, at least about 99%, or about 100% sequence identity to SEQ ID NO:22. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide is IMP321 (eftilagimod alpha). See, e.g., Brignone C, et al., J. Immunol. (2007); 179:4202-4211 and WO2009/044273. In some aspects, eftilagimod alpha is administered at a dose of about 30 mg. In some aspects, eftilagimod alpha is administered subcutaneously at a dose of about 30 mg once about every 2 weeks.

In some aspects, an anti-LAG-3 antibody is used to determine LAG-3 expression. In some aspects, an anti-LAG-3 antibody is selected for its ability to bind to LAG-3 in formalin-fixed, paraffin-embedded (FFPE) tissue specimens. In some aspects, an anti-LAG-3 antibody is capable of binding to LAG-3 in frozen tissues. In some aspects, an anti-LAG-3 antibody is capable of distinguishing membrane bound, cytoplasmic, and/or soluble forms of LAG-3.

In some aspects, an anti-LAG-3 antibody useful for assaying, detecting, and/or quantifying LAG-3 expression in accordance with the methods disclosed herein is the 17B4 mouse IgG1 anti-human LAG-3 monoclonal antibody. See, e.g., Matsuzaki, J et al., PNAS (2010); 107:7875.

In some aspects, the LAG-3 antagonist is formulated for intravenous administration.

In some aspects, the anti-LAG-3 antibody is administered intravenously for about 30 minutes.

In some aspects, the LAG-3 antagonist is administered at a flat dose.

In some aspects, the LAG-3 antagonist is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the LAG-3 antagonist is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the LAG-3 antagonist is administered at a weight-based dose.

In some aspects, the LAG-3 antagonist is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the LAG-3 antagonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, a LAG-3 antagonist as described herein is administered as a monotherapy, i.e., the LAG-3 antagonist is not administered in combination with one or more additional therapeutic agents.

In some aspects, a LAG-3 antagonist as described herein is administered as a combination therapy, i.e., the LAG-3 antagonist is administered in combination with one or more additional therapeutic agents and/or anti-cancer therapies.

### II.B. Combination Therapy

The additional therapeutic agent and/or anti-cancer therapy can comprise any known therapeutic agent or anti-cancer therapy, including a standard of care in the art for the treatment of a subject afflicted with lung cancer. In some aspects, the LAG-3 antagonist is combined with a therapeutic agent and/or therapy described by the NCCN Guidelines® for treatment of NSCLC. See, e.g., therapeutic agents and therapies described at: https://www.cancertherapyadvisor.com/home/cancer-topics/lung-cancer/lung-cancer-treatment-regimens-landing-page/non-small-cell-lung-cancer-treatment-regimens/, last accessed Oct. 23, 2020.

### II.B.1. Anti-Cancer Therapies

In some aspects, the additional anti-cancer therapy comprises a surgery, a radiation therapy, a chemotherapy, an immunotherapy, or any combination thereof. In some aspects, the additional anti-cancer therapy comprises a chemotherapy, including any chemotherapeutic agent disclosed herein. In some aspects, the chemotherapy comprises platinum doublet chemotherapy.

In some aspects, the PDCT comprises a platinum agent in combination with a nucleoside analog, an antimetabolite, a taxane, a vinca alkaloid, or a topisomerase inhibitor.

In some aspects, the platinum agent is cisplatin, carboplatin, oxaliplatin, satraplatin, picoplatin, nedaplatin, triplatin, lipoplatin, or phenanthriplatin.

In some aspects, the nucleoside analog is cytarabine, gemcitabine, lamivudine, entecavir, or telbivudine. In some aspects, the nucleoside analog is gemcitabine.

In some aspects, the antimetabolite is capecitabine, cladribine, clofarabine, cytarabine, floxuridine, fludarabine, fluorouracil, mercaptopurine, methotrexate, pemetrexed, pentostatin, pralatrexate, or thioguanine. In some aspects, the antimetabolite is pemetrexed.

In some aspects, the taxane is paclitaxel, albumin-bound paclitaxel (also called nab-paclitaxel), docetaxel, or cabazitaxel.

In some aspects, the vinca alkaloid is vinblastine, vincristine, vinorelbine, vindesine, vincaminol, vineridine, or vinburnine. In some aspects, the vinca alkaloid is vinorelbine or vinblastine.

In some aspects, the topoisomerase inhibitor is etoposide, mitoxantrone, doxorubicin, irinotecan, topotecan, or camptothecin. In some aspects, the topoisomerase inhibitor is etoposide. In some aspects, the topoisomerase inhibitor is irinotecan.

In some aspects, the PDCT is administered for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 weeks.

In some aspects, the PDCT is administered about every three weeks for about 1, about 2, about 3, about 4, about 5, about 6, about 7, or about 8 cycles. In some aspects, the PDCT is administered about every three weeks for about 1, about 2, about 3, about 4, about 5, or about 6 cycles. In some aspect, the PDCT is administered about every three weeks for about 1, about 2, about 3, or about 4 cycles.

In some aspects, the PDCT is administered for up to about 4, about 5, or about 6 three-week cycles. In some aspects, the PDCT is administered for up to about 4 three-week cycles.

In some aspects, the platinum agent is cisplatin. In some aspects, cisplatin is administered at a dose of about 25 mg/m² to about 150 mg/m², about 50 mg/m² to about 100 mg/m², about 75 mg/m² to about 100 mg/m², or about 75 mg/m² to about 80 mg/m². In some aspects, cisplatin is administered at a dose of about 50 mg/m², about 55 mg/m², about 60 mg/m², about 65 mg/m², about 70 mg/m², about 75 mg/m², about 76 mg/m², about 77 mg/m², about 78 mg/m², about 79 mg/m², about 80 mg/m², about 85 mg/m², about 90 mg/m², about 95 mg/m², or about 100 mg/m². In some aspects, cisplatin is administered intravenously for about 60 minutes. In some aspects, cisplatin is administered on Day 1 of a three-week cycle for up to about 4, about 5, or about 6 cycles.

In some aspects, the platinum agent is carboplatin. In some aspects, carboplatin is administered at a dose for a target area under the concentration-time curve (AUC) of about 1 mg/mL·min to about 10 mg/mL·min. In some aspects, carboplatin is administered in a dose for a target AUC of about 1 mg/mL·min, about 2 mg/mL·min, about 3 mg/mL·min, about 4 mg/mL·min, about 5 mg/mL·min, about 6 mg/mL·min, about 7 mg/mL·min, about 8 mg/mL·min, about 9 mg/mL·min, or about 10 mg/mL·min. In some aspects, carboplatin is administered in a dose for a target AUC of about 2 mg/mL·min. In some aspects, carboplatin is administered in a dose for a target AUC of about 5 mg/mL·min. In some aspects, carboplatin is administered in a dose for a target AUC of about 6 mg/mL·min. In some aspects, carboplatin is administered intravenously for about 30 minutes. In some aspects, carboplatin is administered on Day 1 of a three-week cycle for up to about 4, about 5, or about 6 cycles.

Carboplatin dose can be calculated according to methods known in the art. In some aspects, carboplatin dose is calculated using the Calvert formula as follows:

Carboplatin dose (mg)=target AUC×(CrCl [mL/min]+25).

The Creatine Clearance (CrCl) calculation in the Calvert formula can be determined using the Cockcroft-Gault formula:

Cockcroft-Gault CrCl=[(140−age)×(Weight in kg)×(0.85 if female)]/(72×Cr).

The Cockcroft-Gault formula includes a subject's most recent weight (kg) and most recent serum creatinine (Cr) concentration (mg/dL). In some aspects, if calculation of CrCl by the Cockcroft-Gault formula yields a result of >125 mL/min, then a CrCl is calculated by an alternative formula per institutional standards or capped at 125 mL/min.

In some aspects, the PDCT comprises cisplatin or carboplatin in combination with gemcitabine, paclitaxel, albumin-bound paclitaxel, docetaxel, pemetrexed, vinorelbine, vinblastine, etoposide, or irinotecan.

In some aspects, the PDCT comprises cisplatin or carboplatin in combination with gemcitabine. In some aspects, gemcitabine is administered at a dose of about 1,000 mg/m² to about 1,250 mg/m². In some aspects, gemcitabine is administered at a dose of about 1,000 mg/m², about 1,050 mg/m², about 1,100 mg/m², about 1,150 mg/m², about 1,200 mg/m², or about 1,250 mg/m². In some aspects, gemcitabine is administered intravenously for about 30 minutes. In some aspects, gemcitabine is administered on Days 1, 8, and 15 of a three-week cycle for up to about 4, about 5, or about 6 cycles. In some aspects, gemcitabine is administered on Days 1 and 8 of a three-week cycle for up to about 4, about 5, or about 6 cycles. In some aspects, the PDCT comprises a dose of about 1,000 mg/m² to about 1,250 mg/m² gemcitabine administered intravenously for about 30 minutes on Days 1 and 8 of a three-week cycle for about 4 to about 6 cycles and a dose of about 75 mg/m² to about 80 mg/m² cisplatin administered intravenously for about 60 minutes on Day 1 of each cycle. In some aspects, the PDCT comprises a dose of about 1,000 mg/m² gemcitabine administered intravenously for about 30 minutes on Days 1, 8, and 15 of a three-week cycle for about 4 to about 6 cycles and carboplatin administered intravenously for about 30 minutes in a dose for a target AUC of about 5 mg/mL·min on Day 1 of each cycle.

In some aspects, the PDCT comprises cisplatin or carboplatin in combination with paclitaxel or albumin-bound paclitaxel.

In some aspects, the PDCT comprises cisplatin or carboplatin in combination with paclitaxel. In some aspects, paclitaxel is administered at a dose of about 45 mg/m² to about 200 mg/m². In some aspects, paclitaxel is administered at a dose of about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², or about 200 mg/m². In some aspects, paclitaxel is administered intravenously for about 60 minutes to about 180 minutes. In some aspects, the PDCT comprises a dose of about 200 mg/m² paclitaxel administered intravenously for about 180 minutes on Day 1 of a three-week cycle for about 4 to about 6 cycles and a dose of about 75 mg/m² to about 80 mg/m² cisplatin administered intravenously for about 60 minutes on Day 1 of each cycle. In some aspects, the PDCT comprises a dose of about 135 mg/m² paclitaxel administered intravenously for about 180 minutes on Day 1 of a three-week cycle for about 4 to about 6 cycles and a dose of about 75 mg/m² cisplatin administered intravenously for about 60 minutes on Day 1 of each cycle. In some aspects, the PDCT comprises a dose of about 200 mg/m² paclitaxel administered intravenously for about 180 minutes on Day 1 of a three-week cycle for about 4 to about 6 cycles and carboplatin administered intravenously for about 30 minutes in a dose for a target AUC of about 6 mg/mL·min on Day 1 of each cycle. In some aspects, the PDCT comprises a dose of about 45 mg/m² to about 50 mg/m² paclitaxel administered intravenously for about 60 minutes on Day 1 of a one-week cycle for about 7 cycles and carboplatin administered intravenously for about 30 minutes in a dose for a target AUC of about 2 mg/mL·min on Day 1 of each cycle.

In some aspects, the PDCT comprises cisplatin or carboplatin in combination with albumin-bound paclitaxel. In some aspects, albumin-bound paclitaxel is administered at a dose of about 100 mg/m². In some aspects, albumin-bound paclitaxel is administered intravenously for about 30 minutes. In some aspects, the PDCT comprises a dose of about 100 mg/m² albumin-bound paclitaxel administered intravenously for about 30 minutes on Days 1, 8, and 15 of a three-week cycle for about 4 cycles and a dose of about 75 mg/m² to about 80 mg/m² cisplatin administered intravenously for about 60 minutes on Day 1 of each cycle. In some aspects, the PDCT comprises a dose of about 100 mg/m² albumin-bound paclitaxel administered intravenously for about 30 minutes on Days 1, 8, and 15 of a three-week cycle for about 4 to about 6 cycles and carboplatin administered intravenously for about 30 minutes in a dose for a target AUC of about 6 mg/mL·min on Day 1 of each cycle.

In some aspects, the PDCT comprises cisplatin or carboplatin in combination with docetaxel. In some aspects, docetaxel is administered at a dose of about 75 mg/m². In some aspects, docetaxel is administered intravenously for about 60 minutes. In some aspects, the PDCT comprises a dose of about 75 mg/m² docetaxel administered intravenously for about 60 minutes on Day 1 of a three-week cycle for about 4 to about 6 cycles and a dose of about 75 mg/m² cisplatin administered intravenously for about 60 minutes on Day 1 of each cycle. In some aspects, the PDCT comprises a dose of about 75 mg/m² docetaxel administered intravenously for about 60 minutes on Day 1 of a three-week cycle for about 4 to about 6 cycles and carboplatin administered intravenously for about 30 minutes in a dose for a target AUC of about 6 mg/mL·min on Day 1 of each cycle.

In some aspects, the PDCT comprises cisplatin or carboplatin in combination with pemetrexed. In some aspects, pemetrexed is administered at a dose of about 500 mg/m². In some aspects, pemetrexed is administered intravenously for about 10 minutes. In some aspects, the PDCT comprises a dose of about 500 mg/m² pemetrexed administered intravenously for about 10 minutes on Day 1 of a three-week cycle for about 4 to about 6 cycles and a dose of about 75 mg/m² cisplatin administered intravenously for about 60 minutes on Day 1 of each cycle. In some aspects, the PDCT comprises a dose of about 500 mg/m² pemetrexed administered intravenously for about 10 minutes on Day 1 of a three-week cycle for about 3 cycles and a dose of about 75 mg/m² cisplatin administered intravenously for about 60 minutes on Day 1 of each cycle. In some aspects, the PDCT comprises a dose of about 500 mg/m² pemetrexed administered intravenously for about 60 minutes on Day 1 of a three-week cycle for about 4 to about 6 cycles and carboplatin administered intravenously for about 30 minutes in a dose for a target AUC of about 6 mg/mL·min on Day 1 of each cycle. In some aspects, the PDCT comprises a dose of about 500 mg/m² pemetrexed administered intravenously for about 60 minutes on Day 1 of a three-week cycle for about 4 to about 6 cycles and carboplatin administered intravenously for about 30 minutes in a dose for a target AUC of about 5 mg/mL·min on Day 1 of each cycle.

In some aspects, the PDCT comprises cisplatin or carboplatin in combination with etoposide. In some aspects, etoposide is administered at a dose of about 50 mg/m² to about 100 mg/m². In some aspects, etoposide is administered intravenously for about 30 minutes to about 60 minutes. In some aspects, the PDCT comprises a dose of about 100 mg/m² etoposide administered intravenously for about 60 minutes on Days 1-3 of a three-week cycle for about 4 to about 6 cycles and a dose of about 100 mg/m² cisplatin administered intravenously for about 60 minutes on Day 1 of each cycle. In some aspects, the PDCT comprises a dose of about 100 mg/m² etoposide administered intravenously for about 60 minutes on Days 1-3 of a four-week cycle for about 4 cycles and a dose of about 100 mg/m² cisplatin administered intravenously for about 60 minutes on Day 1 of each cycle. In some aspects, the PDCT comprises a dose of about 50 mg/m² etoposide administered intravenously for about 60 minutes on Days 1-5 of a four-week cycle for about 2 cycles and a dose of about 50 mg/m² cisplatin administered intravenously for about 60 minutes on Days 1 and 8 of each cycle. In some aspects, the PDCT comprises a dose of about 100 mg/m² etoposide administered intravenously for about 30 minutes on Days 1-3 of a three-week cycle for about 4 to about 6 cycles and carboplatin administered intravenously for about 30 minutes in a dose for a target AUC of about 5 mg/mL·min on Day 1 of each cycle.

In some aspects, the PDCT comprises cisplatin and vinorelbine. In some aspects, vinorelbine is administered at a dose of about 25 mg/m² to about 30 mg/m². In some aspects, vinorelbine is administered intravenously for about 5 minutes to about 10 minutes. In some aspects, the PDCT comprises a dose of about 25 mg/m² to about 30 mg/m² vinorelbine administered intravenously for about 5 minutes to about 10 minutes on Days 1 and 8 of a three-week cycle for about 4 cycles and a dose of about 75 mg/m² to about 80 mg/m² cisplatin administered intravenously for about 60 minutes on Day 1 of each cycle. In some aspects, the PDCT comprises a dose of about 25 mg/m² vinorelbine administered intravenously for about 5 minutes to about 10 minutes on Days 1, 8, 15, and 22 of a four-week cycle for about 4 cycles and a dose of about 50 mg/m² cisplatin administered intravenously for about 60 minutes on Days 1 and 8 of each cycle. In some aspects, the PDCT comprises a dose of about 30 mg/m² vinorelbine administered intravenously for about 5 minutes to about 10 minutes on Days 1, 8, 15, and 22 of a four-week cycle for about 4 cycles and a dose of about 100 mg/m² cisplatin administered intravenously for about 60 minutes on Day 1 of each cycle.

In some aspects, the PDCT comprises cisplatin and vinblastine. In some aspects, vinblastine is administered at a dose of about 5 mg/m². In some aspects, vinblastine is administered intravenously for about 5 minutes to about 10 minutes. In some aspects, the PDCT comprises a dose of about 5 mg/m² vinblastine administered intravenously for about 5 minutes to about 10 minutes on Days 1, 8, 15, 22, and 29 of a 35-day cycle and a dose of about 100 mg/m² cisplatin administered intravenously for about 60 minutes on Days 1 and 29 of the cycle.

In some aspects, the PDCT is administered in combination with bevacizumab (also known as AVASTIN®). In some aspects, bevacizumab is administered intravenously at a dose of about 15 mg/kg on Day 1 of a three-week cycle for about 6 cycles, paclitaxel is administered intravenously at a dose of about 200 mg/m² for about 180 minutes on Day 1 of each cycle, and carboplatin is administered intravenously for about 30 minutes in a dose for a target AUC of about 6 mg/mL·min on Day 1 of each cycle. In some aspects, bevacizumab is administered intravenously at a dose of about 15 mg/kg on Day 1 of a three-week cycle for about 4 to about 6 cycles, pemetrexed is administered intravenously at a dose of about 500 mg/m² for about 10 minutes on Day 1 of each cycle, and carboplatin is administered intravenously for about 30 minutes in a dose for a target AUC of about 6 mg/mL·min on Day 1 of each cycle. In some aspects, bevacizumab is administered intravenously at a dose of about 7.5 mg/kg on Day 1 of a three-week cycle for about 4 to about 6 cycles, pemetrexed is administered intravenously at a dose of about 500 mg/m² for about 10 minutes on Day 1 of each cycle, and cisplatin is administered intravenously at a dose of about 75 mg/m² for about 60 minutes on Day 1 of each cycle.

### II.B.2. Therapeutic Agents

In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor comprises sorafenib (e.g., sorafenib tosylate, also known as NEXAVAR®), lenvatinib (e.g., lenvatinib mesylate, also known as LENVIMA®), regorafenib (e.g., STIVARGA®), cabozantinib (e.g., cabozantinib S-malate, also known as CABOMETYX®), sunitinib (e.g., sunitinib malate, also known as SUTENT®), brivanib, linifanib, pemigatinib (also known as PEMAZYRE™), everolimus (also known as AFINITOR® or ZORTRESS®), gefitinib (IRESSA®, a small-molecule TKI of EGFR), imatinib (e.g., imatinib mesylate), lapatinib (e.g., lapatinib ditosylate, also known as TYKERB®), nilotinib (e.g., nilotinib hydrochloride, also known as TASIGNA®), pazopanib (e.g., pazopanib hydrochloride, also known as VOTRIENT®), temsirolimus (also known as TORISEL®), erlotinib (e.g., erlotinib hydrochloride, also known as TARCEVA®, a small-molecule TKI of EGFR), afatinib (GILOTRIF®, a small-molecule TKI of EGFR), dacomitinib (VIZIMPRO®, a small-molecule TKI of EGFR), osimeritinb (TAGRISSO®, a small-molecule TKI of EGFR), alectinib (ALECENSA®, a small-molecule TKI of ALK), ceritinib (ZYKADIA®, a small-molecule TKI of ALK and ROS-1), brigatinib (ALUNBRIG®, a small-molecule TKI of ALK), crizotinib (XALKORI®, a small-molecule TKI of ALK and ROS-1), lorlatinib (LORBRENA®, a small-molecule TKI of ALK and ROS-1), entrectinib (ROZLYTREK®, a small-molecule TKI of ROS-1 and NTRK), dabrafenib (TAFINLAR®, a small-molecule TKI of BRAF) trametinib (MEKINIST®, a small-molecule TKI of BRAF), vemurafenib (ZELBORAF®, a small-molecule TKI of BRAF), larotrectinib (ROZLYTREK®, a small-molecule TKI of NTRK), or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-IA), a epidermal growth factor (EGF), EGFR, or any combination thereof. In some aspects, the anti-angiogenesis agent comprises bevacizumab (also known as AVASTIN®), ramucirumab (also known as CYRAMZA®), aflibercept (also known as EYLEA® or ZALTRAP®), tanibirumab, olaratumab (also known as LARTRUVO™), nesvacumab, AMG780, MEDI3617, vanucizumab, rilotumumab, ficlatuzumab, TAK-701, onartuzumab, emibetuzumab, or any combination thereof.

In some aspects, the anti-angiogenesis agent is bevacizumab. In some aspects, bevacizumab is administered at a dose of about 15 mg/kg. In some aspects, bevacizumab is administered at a dose of about 15 mg/kg on Day 1 of a three-week cycle.

In some aspects, the checkpoint stimulator comprises an agonist of B7-1, B7-2, CD28, 4-1BB (CD137), 4-1BBL, GITR, inducible T cell co-stimulator (ICOS), ICOS-L, OX40, OX40L, CD70, CD27, CD40, death receptor 3 (DR3), CD28H, or any combination thereof.

In some aspects, the chemotherapeutic agent comprises an alkylating agent, an antimetabolite, an antineoplastic antibiotic, a mitotic inhibitor, a hormone or hormone modulator, a protein tyrosine kinase inhibitor, an epidermal growth factor inhibitor, a proteasome inhibitor, other neoplastic agent, or any combination thereof.

In some aspects, the immunotherapeutic agent comprises an antibody that specifically binds to EGFR (e.g., cetuximab (ERBITUX®)), ALK, ROS-1, NTRK, BRAF, ICOS, CD137 (4-1BB), CD134 (OX40), NKG2A, CD27, CD96, GITR, Herpes Virus Entry Mediator (HVEM), PD-1, PD-L1, CTLA-4, BTLA, TIM-3, A2aR, Killer cell Lectin-like Receptor G1 (KLRG-1), Natural Killer Cell Receptor 2B4 (CD244), CD160, TIGIT, VISTA, KIR, TGFβ, IL-10, IL-8, B7-H4, Fas ligand, CSF1R, CXCR4, mesothelin, CEACAM-1, CD52, HER2, MICA, MICB, or any combination thereof.

In some aspects, the platinum agent comprises cisplatin, carboplatin, oxaliplatin, satraplatin, picoplatin, nedaplatin, triplatin (e.g., triplatin tetranitrate), lipoplatin, phenanthriplatin, or any combination thereof.

In some aspects, the alkylating agent comprises altretamine, bendamustine, busulfan, carboplatin, carmustine, chlorambucil, cisplatin, cyclophosphamide, dacarbazine, ifosfamide, lomustine, mechlorethamine, melphalan, oxaliplatin, procarbazine, streptozocin, temozolomide, thiotepa, or any combination thereof.

In some aspects, the taxane comprises paclitaxel, albumin-bound paclitaxel (i.e., nab-paclitaxel), docetaxel, cabazitaxel, or any combination thereof.

In some aspects, the taxane comprises paclitaxel. In some aspects, paclitaxel is administered intravenously at a dose of about 200 mg/m² to about 225 mg/m² for about 180 minutes on Day 1 of a three-week cycle for about 4 to about 6 cycles. In some aspects, paclitaxel is administered intravenously at a dose of about 80 mg/m² for about 60 minutes on Days 1, 8, and 15 of a four-week cycle for about 4 to about 6 cycles.

In some aspects, the taxane comprises albumin-bound paclitaxel. In some aspects, albumin-bound paclitaxel is administered intravenously at a dose of about 260 mg/m² for about 30 minutes on Day 1 of a three-week cycle for about 4 to about 6 cycles. In some aspects, albumin-bound paclitaxel is administered intravenously at a dose of about 125 mg/m² for about 30 minutes on Days 1, 8, and 15 of a four-week cycle for about 4 to about 6 cycles.

In some aspects, the taxane comprises docetaxel. In some aspects, docetaxel is administered intravenously at a dose of about 75 mg/m² for about 60 minutes on Day 1 of a three-week cycle. In some aspects, docetaxel is administered intravenously at a dose of about 75 mg/m² for about 60 minutes on Day 1 of a three-week cycle for about 4 to about 6 cycles.

In some aspects, the nucleoside analog comprises cytarabine, gemcitabine, lamivudine, entecavir, telbivudine, or any combination thereof.

In some aspects, the nucleoside analog is gemcitabine. In some aspects, gemcitabine is administered intravenously at a dose of about 1,000 mg/m² to about 1,250 mg/m² for about 30 minutes on Days 1, 8, and 15 of a four-week cycle. In some aspects, gemcitabine is administered intravenously at a dose of about 1,000 mg/m² to about 1,250 mg/m² for about 30 minutes on Days 1, 8, and 15 of a four-week cycle for about 4 to about 6 cycles. In some aspects, gemcitabine is administered intravenously at a dose of about 1,250 mg/m² for about 30 minutes on Days 1 and 8 of a three-week cycle. In some aspects, gemcitabine is administered intravenously at a dose of about 1,250 mg/m² for about 30 minutes on Days 1 and 8 of a three-week cycle for about 4 to about 6 cycles.

In some aspects, the antimetabolite comprises capecitabine, cladribine, clofarabine, cytarabine, floxuridine, fludarabine, fluorouracil, mercaptopurine, methotrexate, pemetrexed, pentostatin, pralatrexate, thioguanine, or any combination thereof.

In some aspects, the antimetabolite is pemetrexed. In some aspects, pemetrexed is administered intravenously at a dose of about 500 mg/m² for about 10 minutes on Day 1 of a three-week cycle. In some aspects, pemetrexed is administered intravenously at a dose of about 500 mg/m² for about 10 minutes on Day 1 of a three-week cycle for about 4 to about 6 cycles.

In some embodiments, the topoisomerase inhibitor comprises etoposide, mitoxantrone, doxorubicin, irinotecan, topotecan, camptothecin, or any combination thereof.

In some aspects, the anthracycline is doxorubicin, daunorubicin, epirubicin, idarubicin, or any combination thereof.

In some aspects, the vinca alkaloid is vinblastine, vincristine, vinorelbine, vindesine, vincaminol, vineridine, vinburnine, or any combination thereof.

In some aspects, the anti-cancer agent comprises gemcitabine and docetaxel. In some aspects, gemcitabine is administered intravenously at a dose of about 1,000 mg/m² to about 1,250 mg/m² for about 30 minutes on Days 1 and 8 of a three-week cycle for about 4 to about 6 cycles and docetaxel is administered intravenously at a dose of about 85 mg/m² for about 30 minutes to about 60 minutes on Day 8 of each cycle.

In some aspects, the anti-cancer agent comprises gemcitabine and vinorelbine. In some aspects, gemcitabine is administered intravenously at a dose of about 1,000 mg/m² for about 30 minutes on Days 1 and 8 of a three-week cycle for about 4 to about 6 cycles and vinorelbine is administered intravenously at a dose of about 25 mg/m² for about 5 minutes to about 10 minutes on Days 1 and 8 of each cycle.

In some aspects, the anti-cancer agent comprises ramucirumab and docetaxel. In some aspects, ramucirumab is administered intravenously at a dose of about 10 mg/kg for about 60 minutes on Day 1 of a three-week cycle and docetaxel is administered intravenously at a dose of about 75 mg/m² for about 60 minutes on Day 1 of each cycle.

In some aspects, the anti-cancer agent comprises bevacizumab and pemetrexed. In some aspects, bevacizumab is administered intravenously at a dose of about 7.5 mg/kg to about 15 mg/kg for about 10 minutes on Day 1 of a three-week cycle and pemetrexed is administered intravenously at a dose of about 500 mg/m² for about 10 minutes on Day 1 of each cycle.

### II.B.3. Checkpoint Inhibitors

In some aspects, the anti-cancer agent that is administered as an additional therapeutic agent in the methods of the disclosure is a checkpoint inhibitor.

In some aspects, the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor (e.g., an indoleamine 2,3-dioxygenase 1 (IDO1) inhibitor, epacadostat (INCB24360), navoximod (GDC-0919), or linrodostat (BMS-986205), including a linrodostat salt such as, for example, linrodostat mesylate), a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-0) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

In some aspects, the checkpoint inhibitor is formulated for intravenous administration.

In some aspects, the LAG-3 antagonist and the checkpoint inhibitor are formulated separately. In some aspects, each checkpoint inhibitor is formulated separately when the checkpoint inhibitor comprises more than one checkpoint inhibitor. In some aspects, the checkpoint inhibitor is administered before the LAG-3 antagonist. In some aspects, the LAG-3 antagonist is administered before the checkpoint inhibitor.

In some aspects, the LAG-3 antagonist and the checkpoint inhibitor are formulated together. In some aspects, two or more checkpoint inhibitors are formulated together when the checkpoint inhibitor comprises more than one checkpoint inhibitor.

In some aspects, the LAG-3 antagonist and the checkpoint inhibitor are administered concurrently.

In some aspects, the checkpoint inhibitor is administered at a flat dose.

In some aspects, the checkpoint inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the checkpoint inhibitor is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the checkpoint inhibitor is administered as a weight-based dose.

In some aspects, the checkpoint inhibitor is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the checkpoint inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the checkpoint inhibitor is administered every one week, every two weeks, every three weeks, every four weeks, every five weeks, every six weeks, every seven weeks, every eight weeks, every nine weeks, every ten weeks, every eleven weeks, or every twelve weeks.

In some aspects, each dose of the LAG-3 antagonist and/or the checkpoint inhibitor is administered in a constant amount.

In some aspects, each dose of the LAG-3 antagonist and/or the checkpoint inhibitor is administered in a varying amount. For example, in some aspects, the maintenance (or follow-on) dose of the LAG-3 antagonist and/or the checkpoint inhibitor can be higher or the same as the loading dose which is first administered. In some aspects, the maintenance dose of the LAG-3 antagonist and/or the checkpoint inhibitor can be lower or the same as the loading dose.

II.B.3.a. PD-1 Pathway Inhibitors

In some aspects, the checkpoint inhibitor for use in the methods of the disclosure comprises a PD-1 pathway inhibitor.

In some aspects the PD-1 pathway inhibitor is a PD-1 inhibitor and/or a PD-L1 inhibitor.

In some aspects, the PD-1 inhibitor and/or PD-L1 inhibitor is a small molecule.

In some aspects, the PD-1 inhibitor and/or PD-L1 inhibitor is a millamolecule.

In some aspects, the PD-1 inhibitor and/or PD-L1 inhibitor is a macrocyclic peptide.

In certain aspects, the PD-1 inhibitor and/or PD-L1 inhibitor is BMS-986189.

In some aspects, the PD-1 inhibitor is an inhibitor disclosed in International Publication No. WO2014/151634, which is incorporated by reference herein in its entirety.

In some aspects, the PD-1 inhibitor is INCMGA00012 (Insight Pharmaceuticals).

In some aspects, the PD-1 inhibitor comprises a combination of an anti-PD-1 antibody disclosed herein and a PD-1 small molecule inhibitor.

In some aspects, the PD-L1 inhibitor comprises a millamolecule having a formula set forth in formula (I):

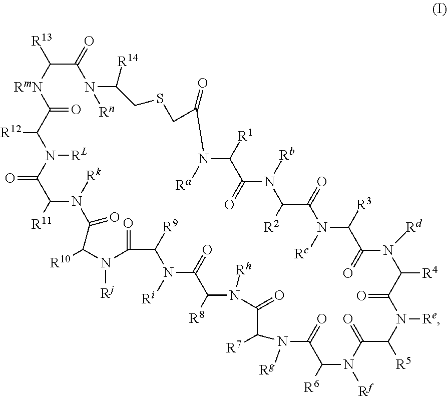

wherein R¹-R¹³ are amino acid side chains, R^{a}-Rⁿ are hydrogen, methyl, or form a ring with a vicinal R group, and R¹⁴ is —C(O)NHR¹⁵, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

In some aspects, the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety.

In some aspects, the PD-1 pathway inhibitor is a soluble PD-L2 polypeptide. In some aspects, the soluble PD-L2 polypeptide is a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224 (see, e.g., US 2013/0017199).

In some aspects, the PD-1 pathway inhibitor is an anti-PD-1 antibody and/or an anti-PD-L1 antibody.

II.B.3.a.i. Anti-PD-1 Antibodies

Anti-PD-1 antibodies that are known in the art can be used in the methods of the disclosure. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-7 production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies that can be used in the methods of the disclosure have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Anti-PD-1 antibodies that can be used in the methods of the disclosure include nivolumab (also known as OPDIVO®, 5C4, BMS-936558, MDX-1106, and ONO-4538), pembrolizumab (Merck; also known as KEYTRUDA®, lambrolizumab, and MK3475; see WO 2008/156712), PDR001 (Novartis; also known as spartalizumab; see WO 2015/112900 and U.S. Pat. No. 9,683,048), MEDI-0680 (AstraZeneca; also known as AMP-514; see WO 2012/145493), TSR-042 (Tesaro Biopharmaceutical; also known as ANB011 or dostarlimab; see WO 2014/179664), cemiplimab (Regeneron; also known as LIBTAYO® or REGN2810; see WO 2015/112800 and U.S. Pat. No. 9,987,500), JS001 (TAIZHOU JUNSHI PHARMA; also known as toripalimab; see Si-Yang Liu et al., J. Hematol. Oncol. 10:136 (2017)), PF-06801591 (Pfizer; also known as sasanlimab; US 2016/0159905), BGB-A317 (Beigene; also known as tislelizumab; see WO 2015/35606 and US 2015/0079109), BI 754091 (Boehringer Ingelheim; see Zettl M et al., Cancer. Res. (2018); 78(13 Suppl):Abstract 4558), INCSHR1210 (Jiangsu Hengrui Medicine; also known as SHR-1210 or camrelizumab; see WO 2015/085847; Si-Yang Liu et al., J. Hematol. Oncol. 10:136 (2017)), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), IBI308 (Innovent; also known as sintilimab; see WO 2017/024465, WO 2017/025016, WO 2017/132825, and WO 2017/133540), and SSI-361 (Lyvgen Biopharma Holdings Limited, US 2018/0346569).

Anti-PD-1 antibodies that can be used in the methods of the disclosure also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab.

In some aspects, the antibodies that cross-compete for binding to human PD-1 with, or bind to the same epitope region as, any anti-PD-1 antibody disclosed herein, e.g., nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies that can be used in the methods of the disclosure also include antigen-binding portions of any of the above full-length antibodies.

Anti-PD-1 antibodies that can be used in the methods of the disclosure are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In some aspects, the anti-PD-1 antibody is a full-length antibody. In some aspects, the anti-PD-1 antibody is a monoclonal, human, humanized, chimeric, or multispecific antibody. In some aspects, the multispecific antibody is a DART, a DVD-Ig, or bispecific antibody.

In some aspects, the anti-PD-1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-1 antibody is nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

In some aspects, the anti-PD-1 antibody is formulated for intravenous administration.

In some aspects, the anti-PD-1 antibody is administered intravenously for about 30 minutes.

In some aspects, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In some aspects, nivolumab is administered at a flat dose of about 240 mg once about every 2 weeks. In some aspects, nivolumab is administered at a flat dose of about 240 mg once about every 3 weeks. In some aspects, nivolumab is administered at a flat dose of about 360 mg once about every 3 weeks. In some aspects, nivolumab is administered at a flat dose of about 480 mg once about every 4 weeks.

In some aspects, nivolumab is administered intravenously at a dose of about 240 mg for about 30 minutes on Day 1 of a two-week cycle.

In some aspects, nivolumab is administered intravenously at a dose of about 480 mg for about 30 minutes on Day 1 of a four-week cycle.

In some aspects, the methods of the disclosure comprise an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

In some aspects, the methods of the disclosure comprise an anti-PD-1 antibody comprising: (a) a heavy chain variable region CDR1 comprising the sequence set forth in SEQ ID NO:15; (b) a heavy chain variable region CDR2 comprising the sequence set forth in SEQ ID NO: 16; (c) a heavy chain variable region CDR3 comprising the sequence set forth in SEQ ID NO: 17; (d) a light chain variable region CDR1 comprising the sequence set forth in SEQ ID NO:18; (e) a light chain variable region CDR2 comprising the sequence set forth in SEQ ID NO:19; and (f) a light chain variable region CDR3 comprising the sequence set forth in SEQ ID NO:20.

In some aspects, the methods of the disclosure comprise an anti-PD-1 antibody comprising heavy and light chain variable regions comprising the sequences set forth in SEQ ID NOs:13 and 14, respectively.

In some aspects, the methods of the disclosure comprise an anti-PD-1 antibody comprising heavy and light chains comprising the sequences as set forth in SEQ ID NOs:11 and 12, respectively.

In some aspects, the methods of the disclosure comprise a combination of relatlimab and nivolumab.

In some aspects, the methods of the disclosure comprise: (a) an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4; and (b) an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

In some aspects, the methods of the disclosure comprise: (a) an anti-LAG-3 antibody comprising a heavy chain variable region CDR1, CDR2, and CDR3 comprising the sequence set forth in SEQ ID NO:5, SEQ ID NO:6, and SEQ ID NO:7, respectively, and a light chain variable region CDR1, CDR2, and CDR3 comprising the sequence set forth in SEQ ID NO:8, SEQ ID NO:9, and SEQ ID NO:10, respectively, and (b) an anti-PD-1 antibody comprising a heavy chain variable region CDR1, CDR2, and CDR3 comprising the sequence set forth in SEQ ID NO:15, SEQ ID NO:16, and SEQ ID NO:17, respectively, and a light chain variable region CDR1, CDR2, and CDR3 comprising the sequence set forth in SEQ ID NO:18, SEQ ID NO:19, and SEQ ID NO:20, respectively.

In some aspects, the methods of the disclosure comprise: (a) an anti-LAG-3 antibody comprising heavy and light chain variable regions comprising the sequences set forth in SEQ ID NOs:3 and 4, respectively, and (b) an anti-PD-1 antibody comprising heavy and light chain variable regions comprising the sequences set forth in SEQ ID NOs:13 and 14, respectively.

In some aspects, the methods of the disclosure comprise: (a) an anti-LAG-3 antibody comprising heavy and light chains comprising the sequences set forth in SEQ ID NOs:1 and 2, respectively, and (b) an anti-PD-1 antibody comprises heavy and light chains comprising the sequences as set forth in SEQ ID NOs:11 and 12, respectively.

In some aspects, the methods of the disclosure comprise: (a) an anti-LAG-3 antibody comprising heavy and light chains comprising the sequences set forth in SEQ ID NOs:21 and 2, respectively, and (b) an anti-PD-1 antibody comprises heavy and light chains comprising the sequences as set forth in SEQ ID NOs:11 and 12, respectively.

In some aspects, the anti-PD-1 antibody is pembrolizumab. Pembrolizumab is a humanized monoclonal IgG4 (S228P) antibody directed against human cell surface receptor PD-1. Pembrolizumab is described, for example, in U.S. Pat. Nos. 8,354,509 and 8,900,587.

In some aspects, pembrolizumab is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 6 weeks. In some aspects, pembrolizumab is administered at a flat dose of about 300 mg once about every 4-5 weeks.

In some aspects, pembrolizumab is administered intravenously at a dose of about 200 mg on Day 1, then once about every 3 weeks. In some aspects, pembrolizumab is administered for up to 35 cycles. In some aspects, pembrolizumab is administered intravenously at a dose of about 200 mg for about 30 minutes on Day 1 of a three-week cycle for up to 35 cycles.

In some aspects, pembrolizumab is administered intravenously at a dose of about 200 mg for about 30 minutes on Day 1 of a three-week cycle for up to 35 cycles and pemetrexed is administered intravenously at a dose of about 500 mg/m² for about 10 minutes on Day 1 of each cycle, followed by maintenance therapy with pemetrexed at a dose of about 500 mg/m² administered intravenously on Day 1 of a three-week cycle. In some embodiments, the maintenance therapy continues until disease progression or unacceptable toxicity.

In some aspects, pembrolizumab is administered intravenously at a dose of about 200 mg for about 30 minutes on Day 1 of a three week cycle for about 4 to about 6 cycles, pemetrexed is administered intravenously at a dose of about 500 mg/m² for about 10 minutes on Day 1 of each cycle, and carboplatin is administered intravenously for about 30 minutes in a dose for a target AUC of about 5 mg/mL·min on Day 1 of each cycle.

In some aspects, pembrolizumab is administered intravenously at a dose of about 200 mg for about 30 minutes on Day 1 of a three week cycle for about 4 to about 6 cycles, pemetrexed is administered intravenously at a dose of about 500 mg/m² for about 10 minutes on Day 1 of each cycle, and cisplatin is administered intravenously at a dose of about 75 mg/m² for about 60 minutes on Day 1 of each cycle.

In some aspects, pembrolizumab is administered intravenously at a dose of about 200 mg for about 30 minutes on Day 1 of a three week cycle for about 4 cycles, albumin-bound paclitaxel is administered intravenously at a dose of about 100 mg/m² for about 30 minutes on Days 1, 8, and 15 of each cycle, and carboplatin is administered intravenously for about 30 minutes in a dose for a target AUC of about 6 mg/mL·min on Day 1 of each cycle.

In some aspects, pembrolizumab is administered intravenously at a dose of about 200 mg for about 30 minutes on Day 1 of a three week cycle for about 4 cycles, paclitaxel is administered intravenously at a dose of about 200 mg/m² for about 180 minutes on Day 1 of each cycle, and carboplatin is administered intravenously for about 30 minutes in a dose for a target AUC of about 6 mg/mL·min on Day 1 of each cycle.

In some aspects, pembrolizumab is administered intravenously at a dose of about 200 mg for about 30 minutes on Day 1 of a three week cycle for about 4 cycles, albumin-bound paclitaxel is administered intravenously at a dose of about 100 mg/m² for about 30 minutes on Days 1, 8, and 15 of each cycle, and cisplatin is administered intravenously at a dose of about 75 mg/m² to about 80 mg/m² for about 60 minutes on Day 1 of each cycle.

In some aspects, pembrolizumab is administered intravenously at a dose of about 200 mg for about 30 minutes on Day 1 of a three week cycle for about 4 cycles, paclitaxel is administered intravenously at a dose of about 200 mg/m² for about 180 minutes on Day 1 of each cycle, and cisplatin is administered intravenously at a dose of about 75 mg/m² to about 80 mg/m² for about 60 minutes on Day 1 of each cycle.

In some aspects, the methods of the disclosure comprise an anti-PD-1 antibody comprising CDR1, CDR2 and CDR3 domains of the heavy chain variable region having the sequence set forth in SEQ ID NO:79, and CDR1, CDR2 and CDR3 domains of the light chain variable region having the sequence set forth in SEQ ID NO:80.

In some aspects, the methods of the disclosure comprise an anti-PD-1 antibody comprising: (a) a heavy chain variable region CDR1 comprising the sequence set forth in SEQ ID NO:81; (b) a heavy chain variable region CDR2 comprising the sequence set forth in SEQ ID NO:82; (c) a heavy chain variable region CDR3 comprising the sequence set forth in SEQ ID NO:83; (d) a light chain variable region CDR1 comprising the sequence set forth in SEQ ID NO:84; (e) a light chain variable region CDR2 comprising the sequence set forth in SEQ ID NO:85; and (f) a light chain variable region CDR3 comprising the sequence set forth in SEQ ID NO:86.

In some aspects, the methods of the disclosure comprise an anti-PD-1 antibody comprising heavy and light chain variable regions comprising the sequences set forth in SEQ ID NOs:79 and 80, respectively.

In some aspects, the methods of the disclosure comprise an anti-PD-1 antibody comprising heavy and light chains comprising the sequences as set forth in SEQ ID NOs:77 and 78, respectively.

In some aspects, the methods of the disclosure comprise a combination of favezelimab and pembrolizumab. In some aspects, 800 mg of favezelimab and 200 mg of pembrolizumab are administered intravenously on Day 1, then once about every 3 weeks. In some aspects, the combination of favezelimab and pembrolizumab is administered for up to 35 cycles. In some aspects, 800 mg of favezelimab and 200 mg of pembrolizumab are administered intravenously for about 30 minutes on Day 1 of a three-week cycle for up to 35 cycles.

In some aspects, the methods of the disclosure comprise: (a) an anti-LAG-3 antibody comprising CDR1, CDR2 and CDR3 domains of the heavy chain variable region having the sequence set forth in SEQ ID NO:69, and CDR1, CDR2 and CDR3 domains of the light chain variable region having the sequence set forth in SEQ ID NO:70; and (b) an anti-PD-1 antibody comprising CDR1, CDR2 and CDR3 domains of the heavy chain variable region having the sequence set forth in SEQ ID NO:79, and CDR1, CDR2 and CDR3 domains of the light chain variable region having the sequence set forth in SEQ ID NO:80.

In some aspects, the methods of the disclosure comprise: (a) an anti-LAG-3 antibody comprising a heavy chain variable region CDR1, CDR2, and CDR3 comprising the sequence set forth in SEQ ID NO:71, SEQ ID NO:72, and SEQ ID NO:73, respectively, and a light chain variable region CDR1, CDR2, and CDR3 comprising the sequence set forth in SEQ ID NO:74, SEQ ID NO:75, and SEQ ID NO:76, respectively, and (b) an anti-PD-1 antibody comprising a heavy chain variable region CDR1, CDR2, and CDR3 comprising the sequence set forth in SEQ ID NO:81, SEQ ID NO:82, and SEQ ID NO:83, respectively, and a light chain variable region CDR1, CDR2, and CDR3 comprising the sequence set forth in SEQ ID NO:84, SEQ ID NO:85, and SEQ ID NO:86, respectively.

In some aspects, the methods of the disclosure comprise: (a) an anti-LAG-3 antibody comprising heavy and light chain variable regions comprising the sequences set forth in SEQ ID NOs:69 and 70, respectively, and (b) an anti-PD-1 antibody comprising heavy and light chain variable regions comprising the sequences set forth in SEQ ID NOs:79 and 80, respectively.

In some aspects, the methods of the disclosure comprise: (a) an anti-LAG-3 antibody comprising heavy and light chains comprising the sequences set forth in SEQ ID NOs:67 and 68, respectively, and (b) an anti-PD-1 antibody comprises heavy and light chains comprising the sequences as set forth in SEQ ID NOs:77 and 78, respectively.

In some aspects, the anti-PD-1 antibody is cemiplimab (REGN2810). Cemiplimab is described, for example, in WO 2015/112800 and U.S. Pat. No. 9,987,500.

In some aspects, cemiplimab is administered intravenously at a dose of about 3 mg/kg or about 350 mg once about every 3 weeks.

In some aspects, the methods of the disclosure comprise an anti-PD-1 antibody comprising CDR1, CDR2 and CDR3 domains of the heavy chain variable region having the sequence set forth in SEQ ID NO:35, and CDR1, CDR2 and CDR3 domains of the light chain variable region having the sequence set forth in SEQ ID NO:36.

In some aspects, the methods of the disclosure comprise an anti-PD-1 antibody comprising: (a) a heavy chain variable region CDR1 comprising the sequence set forth in SEQ ID NO:37; (b) a heavy chain variable region CDR2 comprising the sequence set forth in SEQ ID NO:38; (c) a heavy chain variable region CDR3 comprising the sequence set forth in SEQ ID NO:39; (d) a light chain variable region CDR1 comprising the sequence set forth in SEQ ID NO:40; (e) a light chain variable region CDR2 comprising the sequence set forth in SEQ ID NO:41; and (f) a light chain variable region CDR3 comprising the sequence set forth in SEQ ID NO:42.

In some aspects, the methods of the disclosure comprise an anti-PD-1 antibody comprising heavy and light chain variable regions comprising the sequences set forth in SEQ ID NOs:35 and 36, respectively.

In some aspects, the methods of the disclosure comprise an anti-PD-1 antibody comprising heavy and light chains comprising the sequences as set forth in SEQ ID NOs:33 and 34, respectively.

In some aspects, the methods of the disclosure comprise a combination of fianlimab and cemiplimab.

In some aspects, the methods of the disclosure comprise: (a) an anti-LAG-3 antibody comprising CDR1, CDR2 and CDR3 domains of the heavy chain variable region having the sequence set forth in SEQ ID NO:25, and CDR1, CDR2 and CDR3 domains of the light chain variable region having the sequence set forth in SEQ ID NO:26; and (b) an anti-PD-1 antibody comprising CDR1, CDR2 and CDR3 domains of the heavy chain variable region having the sequence set forth in SEQ ID NO:35, and CDR1, CDR2 and CDR3 domains of the light chain variable region having the sequence set forth in SEQ ID NO:36.

In some aspects, the methods of the disclosure comprise: (a) an anti-LAG-3 antibody comprising a heavy chain variable region CDR1, CDR2, and CDR3 comprising the sequence set forth in SEQ ID NO:27, SEQ ID NO:28, and SEQ ID NO:29, respectively, and a light chain variable region CDR1, CDR2, and CDR3 comprising the sequence set forth in SEQ ID NO:30, SEQ ID NO:31, and SEQ ID NO:32, respectively, and (b) an anti-PD-1 antibody comprising a heavy chain variable region CDR1, CDR2, and CDR3 comprising the sequence set forth in SEQ ID NO:37, SEQ ID NO:38, and SEQ ID NO:39, respectively, and a light chain variable region CDR1, CDR2, and CDR3 comprising the sequence set forth in SEQ ID NO:40, SEQ ID NO:41, and SEQ ID NO:42, respectively.

In some aspects, the methods of the disclosure comprise: (a) an anti-LAG-3 antibody comprising heavy and light chain variable regions comprising the sequences set forth in SEQ ID NOs:25 and 26, respectively, and (b) an anti-PD-1 antibody comprising heavy and light chain variable regions comprising the sequences set forth in SEQ ID NOs:35 and 36, respectively.

In some aspects, the methods of the disclosure comprise: (a) an anti-LAG-3 antibody comprising heavy and light chains comprising the sequences set forth in SEQ ID NOs:23 and 24, respectively, and (b) an anti-PD-1 antibody comprises heavy and light chains comprising the sequences as set forth in SEQ ID NOs:33 and 34, respectively.

In some aspects, the anti-PD-1 antibody is spartalizumab (PDR001). Spartalizumab is described, for example, in WO 2015/112900 and U.S. Pat. No. 9,683,048.

In some aspects, spartalizumab is administered intravenously at a dose of about 300 mg once about every 3 weeks or 400 mg once about every 4 weeks.

In some aspects, the methods of the disclosure comprise an anti-PD-1 antibody comprising CDR1, CDR2 and CDR3 domains of the heavy chain variable region having the sequence set forth in SEQ ID NO:59, and CDR1, CDR2 and CDR3 domains of the light chain variable region having the sequence set forth in SEQ ID NO:60.

In some aspects, the methods of the disclosure comprise an anti-PD-1 antibody comprising: (a) a heavy chain variable region CDR1 comprising the sequence set forth in SEQ ID NO:61; (b) a heavy chain variable region CDR2 comprising the sequence set forth in SEQ ID NO:62; (c) a heavy chain variable region CDR3 comprising the sequence set forth in SEQ ID NO:63; (d) a light chain variable region CDR1 comprising the sequence set forth in SEQ ID NO:64; (e) a light chain variable region CDR2 comprising the sequence set forth in SEQ ID NO:65; and (f) a light chain variable region CDR3 comprising the sequence set forth in SEQ ID NO:66.

In some aspects, the methods of the disclosure comprise an anti-PD-1 antibody comprising heavy and light chain variable regions comprising the sequences set forth in SEQ ID NOs:59 and 60, respectively.

In some aspects, the methods of the disclosure comprise an anti-PD-1 antibody comprising heavy and light chains comprising the sequences as set forth in SEQ ID NOs:57 and 58, respectively.

In some aspects, the methods of the disclosure comprise a combination of ieramilimab and spartalizumab. In some aspects, ieramilimab is administered intravenously at a dose of about 400 mg once about every three weeks and spartalizumab is administered intravenously at a dose of about 300 mg once about every 3 weeks. In some aspects, ieramilimab is administered intravenously at a dose of about 600 mg once about every four weeks and spartalizumab is administered intravenously at a dose of about 400 mg once about every 4 weeks.

In some aspects, the methods of the disclosure comprise: (a) an anti-LAG-3 antibody comprising CDR1, CDR2 and CDR3 domains of the heavy chain variable region having the sequence set forth in SEQ ID NO:47, and CDR1, CDR2 and CDR3 domains of the light chain variable region having the sequence set forth in SEQ ID NO:49; and (b) an anti-PD-1 antibody comprising CDR1, CDR2 and CDR3 domains of the heavy chain variable region having the sequence set forth in SEQ ID NO:59, and CDR1, CDR2 and CDR3 domains of the light chain variable region having the sequence set forth in SEQ ID NO:60.

In some aspects, the methods of the disclosure comprise: (a) an anti-LAG-3 antibody comprising CDR1, CDR2 and CDR3 domains of the heavy chain variable region having the sequence set forth in SEQ ID NO:48, and CDR1, CDR2 and CDR3 domains of the light chain variable region having the sequence set forth in SEQ ID NO:50; and (b) an anti-PD-1 antibody comprising CDR1, CDR2 and CDR3 domains of the heavy chain variable region having the sequence set forth in SEQ ID NO:59, and CDR1, CDR2 and CDR3 domains of the light chain variable region having the sequence set forth in SEQ ID NO:60.

In some aspects, the methods of the disclosure comprise: (a) an anti-LAG-3 antibody comprising a heavy chain variable region CDR1, CDR2, and CDR3 comprising the sequence set forth in SEQ ID NO:51, SEQ ID NO:52, and SEQ ID NO:53, respectively, and a light chain variable region CDR1, CDR2, and CDR3 comprising the sequence set forth in SEQ ID NO:54, SEQ ID NO:55, and SEQ ID NO:56, respectively, and (b) an anti-PD-1 antibody comprising a heavy chain variable region CDR1, CDR2, and CDR3 comprising the sequence set forth in SEQ ID NO:61, SEQ ID NO:62, and SEQ ID NO:63, respectively, and a light chain variable region CDR1, CDR2, and CDR3 comprising the sequence set forth in SEQ ID NO:64, SEQ ID NO:65, and SEQ ID NO:66, respectively.

In some aspects, the methods of the disclosure comprise: (a) an anti-LAG-3 antibody comprising heavy and light chain variable regions comprising the sequences set forth in SEQ ID NOs:47 and 49, respectively, and (b) an anti-PD-1 antibody comprising heavy and light chain variable regions comprising the sequences set forth in SEQ ID NOs:59 and 60, respectively.

In some aspects, the methods of the disclosure comprise: (a) an anti-LAG-3 antibody comprising heavy and light chain variable regions comprising the sequences set forth in SEQ ID NOs:48 and 50, respectively, and (b) an anti-PD-1 antibody comprising heavy and light chain variable regions comprising the sequences set forth in SEQ ID NOs:59 and 60, respectively.

In some aspects, the methods of the disclosure comprise: (a) an anti-LAG-3 antibody comprising heavy and light chains comprising the sequences set forth in SEQ ID NOs:43 and 45, respectively, and (b) an anti-PD-1 antibody comprises heavy and light chains comprising the sequences as set forth in SEQ ID NOs:57 and 58, respectively.

In some aspects, the methods of the disclosure comprise: (a) an anti-LAG-3 antibody comprising heavy and light chains comprising the sequences set forth in SEQ ID NOs:44 and 46, respectively, and (b) an anti-PD-1 antibody comprises heavy and light chains comprising the sequences as set forth in SEQ ID NOs:57 and 58, respectively.

Provided herein is a method of treating a human subject afflicted with lung cancer, the method comprising administering to the subject: (a) an anti-LAG-3 antibody, and (b) an anti-PD-1 antibody.

In some aspects, the method further comprises administering to the subject a PDCT.

Provided herein is a method of treating a human subject afflicted with Stage IV or recurrent NSCLC that has a squamous histology, the method comprising administering to the subject: (a) an anti-LAG-3 antibody, (b) an anti-PD-1 antibody, and (c) a PDCT.

Provided herein is a method of treating a human subject afflicted with Stage IV or recurrent NSCLC that has a non-squamous histology, the method comprising administering to the subject: (a) an anti-LAG-3 antibody, (b) an anti-PD-1 antibody, and (c) a PDCT.

The anti-LAG-3 antibody and the anti-PD-1 antibodies can be administered at any of the doses or combinations of doses described herein.

In some aspects, the dose of the anti-LAG-3 antibody is 80 mg.

In some aspects, the dose of the anti-LAG-3 antibody is 160 mg.

In some aspects, the dose of the anti-LAG-3 antibody is 360 mg.

In some aspects, the dose of the anti-LAG-3 antibody is 480 mg.

In some aspects, the dose of the anti-LAG-3 antibody is 720 mg.

In some aspects, the dose of the anti-LAG-3 antibody is 800 mg.

In some aspects, the dose of the anti-LAG-3 antibody is 960 mg.

In some aspects, the dose of the anti-PD-1 antibody is 200 mg.

In some aspects, the dose of the anti-PD-1 antibody is 240 mg.

In some aspects, the dose of the anti-PD-1 antibody is 360 mg.

In some aspects, the dose of the anti-PD-1 antibody is 480 mg.

In some aspects, the dose of the anti-LAG-3 antibody is 80 mg and the dose of the anti-PD-1 antibody is 240 mg.

In some aspects, the dose of the anti-LAG-3 antibody is 160 mg and the dose of the anti-PD-1 antibody is 480 mg.

In some aspects, the dose of the anti-LAG-3 antibody is 360 mg and the dose of the anti-PD-1 antibody is 360 mg.

In some aspects, the dose of the anti-LAG-3 antibody is 480 mg and the dose of the anti-PD-1 antibody is 480 mg.

In some aspects, the dose of the anti-LAG-3 antibody is 720 mg and the dose of the anti-PD-1 antibody is 360 mg.

In some aspects, the dose of the anti-LAG-3 antibody is 800 mg and the dose of the anti-PD-1 antibody is 200 mg.

In some aspects, the dose of the anti-LAG-3 antibody is 960 mg and the dose of the anti-PD-1 antibody is 480 mg.

Provided herein is a method of treating a human subject afflicted with lung cancer, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, and (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

Provided herein is a method of treating a human subject afflicted with lung cancer, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

Provided herein is a method of treating a human subject afflicted with Stage IV or recurrent NSCLC that has a squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 200 mg/m² of paclitaxel, wherein the method is a first line therapy.

Provided herein is a method of treating a human subject afflicted with Stage IV or recurrent NSCLC that has a squamous histology, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 200 mg/m² of paclitaxel, wherein the method is a first line therapy.

Provided herein is a method of treating a human subject afflicted with Stage IV or recurrent NSCLC that has a squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 100 mg/m² of albumin-bound paclitaxel, wherein the method is a first line therapy.

Provided herein is a method of treating a human subject afflicted with Stage IV or recurrent NSCLC that has a squamous histology, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 6 mg/mL·min, and (ii) a dose of about 100 mg/m² of albumin-bound paclitaxel, wherein the method is a first line therapy.

Provided herein is a method of treating a human subject afflicted with Stage IV or recurrent NSCLC that has a non-squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 5 mg/mL·min or about 6 mg/mL·min, and (ii) a dose of about 500 mg/m² of pemetrexed, wherein the method is a first line therapy.

Provided herein is a method of treating a human subject afflicted with Stage IV or recurrent NSCLC that has a non-squamous histology, the method comprising administering to the subject: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of carboplatin for a target area under the concentration-time curve of about 5 mg/mL·min or about 6 mg/mL·min, and (ii) a dose of about 500 mg/m² of pemetrexed, wherein the method is a first line therapy.

Provided herein is a method of treating a human subject afflicted with Stage IV or recurrent NSCLC that has a non-squamous histology, the method comprising administering to the subject: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, (b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14, (c) a PDCT comprising: (i) a dose of about 75 mg/m² of cisplatin, and (ii) a dose of about 500 mg/m² of pemetrexed, wherein the method is a first line therapy.

Provided herein is a method of treating a human subject afflicted with Stage IV or recurrent NSCLC that has a non-squamous histology, the method comprising administering to the subject: (a) a dose of about 720 mg of an anti-LAG-3 antibody comprising CDR1, CDR2, and CDR3 domains of the heavy chain variable region having the sequence set forth in SEQ ID NO:3, and CDR1, CDR2, and CDR3 domains of the light chain variable region having the sequence set forth in SEQ ID NO:4, (b) a dose of about 360 mg of an anti-PD-1 antibody comprising CDR1, CDR2, and CDR3 domains of the heavy chain variable region having the sequence set forth in SEQ ID NO:13, and CDR1, CDR2, and CDR3 domains of the light chain variable region having the sequence set forth in SEQ ID NO:14, (c) a PDCT comprising: (i) a dose of about 75 mg/m² of cisplatin, and (ii) a dose of about 500 mg/m² of pemetrexed, wherein the method is a first line therapy.

In some aspects, the anti-LAG-3 antibody and the anti-PD-1 antibody are administered about once every three weeks. In some aspects, the anti-LAG-3 antibody and the anti-PD-1 antibody are administered on Day 1 of every three-week cycle.

In some aspects, the anti-LAG-3 antibody is administered intravenously for about 30 minutes.

In some aspects, the anti-PD-1 antibody is administered intravenously for about 30 minutes.

In some aspects, the composition comprising an anti-LAG-3 antibody and an anti-PD-1 antibody is administered intravenously for about 30 minutes.

In some aspects, the anti-LAG-3 antibody and the anti-PD-1 antibody are administered intravenously from a single intravenous bag for about 30 minutes.

In some aspects, a PDCT is administered every three weeks. In some aspects, the PDCT is administered in a three week cycle for up to about 4 cycles.

In some aspects, the anti-LAG-3 antibody and the anti-PD-1 antibody are administered prior to a PDCT.

In some aspects, the PDCT comprises carboplatin and paclitaxel. In some aspects, paclitaxel is administered for about 180 minutes on Day 1 of each cycle followed by carboplatin administered intravenously for about 30 minutes on Day 1 of each cycle. In some aspects, the NSCLC has a squamous histology.

In some aspects, the PDCT comprises carboplatin and albumin-bound paclitaxel. In some aspects, albumin-bound paclitaxel is administered for about 30 minutes on Days 1, 8, and 15 of each cycle followed by carboplatin administered intravenously for about 30 minutes on Day 1 of each cycle. In some aspects, the NSCLC has a squamous histology.

In some aspects, the PDCT comprises carboplatin and pemetrexed. In some aspects, pemetrexed is administered for about 10 minutes on Day 1 of each cycle followed by carboplatin administered intravenously for about 30 minutes on Day 1 of each cycle. In some aspects, pemetrexed is administered at a maintenance dose alone or in combination with the anti-LAG-3 and anti-PD-1 antibodies in subjects who have stable disease or a response following administration of the PDCT for about 4 three-week cycles. In some aspects, the maintenance dose of pemetrexed is 500 mg/m². In some aspects, the maintenance dose is administered on Day 1 of a three-week cycle. In some aspects, the maintenance dose continues until disease progression or unacceptable toxicity. In some aspects, the NSCLC has a non-squamous histology.

In some aspects, the PDCT comprises cisplatin and pemetrexed. In some aspects, pemetrexed is administered for about 10 minutes on Day 1 of each cycle followed by cisplatin administered intravenously for about 30 minutes on Day 1 of each cycle. In some aspects, pemetrexed is administered at a maintenance dose alone or in combination with the anti-LAG-3 and anti-PD-1 antibodies in subjects who have stable disease or a response following administration of the PDCT for about 4 cycles. In some aspects, the maintenance dose of pemetrexed is 500 mg/m². In some aspects, the maintenance dose is administered on Day 1 of a three-week cycle. In some aspects, the maintenance dose continues until disease progression or unacceptable toxicity. In some aspects, the NSCLC has a non-squamous histology.

II.B.3.a.ii. Anti-PD-L1 Antibodies

Anti-PD-L1 antibodies that are known in the art can be used in the methods of the disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-7 production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

Anti-PD-L1 antibodies that can be used in the methods of the disclosure include BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IVFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), ICO 36, FAZ053 (Novartis), and CK-301 (Checkpoint Therapeutics; see Gorelik et al., AACR:Abstract 4606 (April 2016)).

Anti-PD-L1 antibodies that can be used in the methods of the disclosure also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. In certain aspects, the antibodies that cross-compete for binding to human PD-L1 with, or bind to the same epitope region as, any anti-PD-L1 antibody disclosed herein, e.g., atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies that can be used in the methods of the disclosure also include antigen-binding portions of any of the above full-length antibodies.

Anti-PD-L1 antibodies that can be used in the methods of the disclosure are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

In some aspects, an anti-PD-L1 antibody is substituted for the anti-PD-1 antibody in any of the methods disclosed herein.

In some aspects, the anti-PD-L1 antibody is a full-length antibody.

In some aspects, the anti-PD-L1 antibody is a monoclonal, human, humanized, chimeric, or multispecific antibody. In some aspects, the multispecific antibody is a DART, a DVD-Ig, or bispecific antibody.

In some aspects, the anti-PD-L1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-L1 antibody is BMS-936559, atezolizumab, durvalumab, avelumab, STI-1014, CX-072, KN035, LY3300054, BGB-A333, ICO 36, FAZ053, CK-301, or comprises an antigen binding portion thereof.

In some aspects, the PD-L1 antibody is atezolizumab. Atezolizumab is a fully humanized IgG1 monoclonal anti-PD-L1 antibody. In some aspects, atezolizumab is administered as a flat dose of about 800 mg once about every 2 weeks. In some aspects, atezolizumab is administered as a flat dose of about 840 mg once about every 2 weeks.

In some aspects, atezolizumab is administered intravenously at a dose of about 1,200 mg on Day 1 of a three-week cycle.

In some aspects, atezolizumab is administered intravenously at a dose of about 1,200 mg on Day 1 of a three-week cycle, and bevacizumab is administered at a dose of about 15 mg/kg on Day 1 of each cycle.

In some aspects, atezolizumab is administered intravenously at a dose of about 1,200 mg on Day 1 of a three-week cycle for about 4 to about 6 cycles, bevacizumab is administered at a dose of about 15 mg/kg on Day 1 of each cycle, paclitaxel is administered intravenously at a dose of about 200 mg/m² for about 180 minutes on Day 1 of each cycle, and carboplatin is administered intravenously for about 30 minutes in a dose for a target AUC of about 6 mg/mL·min on Day 1 of each cycle.

In some aspects, the PD-L1 antibody is durvalumab. Durvalumab is a human IgG1 kappa monoclonal anti-PD-L1 antibody. In some aspects, durvalumab is administered at a dose of about 10 mg/kg once about every 2 weeks. In some aspects, durvalumab is administered at a dose of about 10 mg/kg once about every 2 weeks for up to 12 months. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the PD-L1 antibody is avelumab. Avelumab is a human IgG1 lambda monoclonal anti-PD-L1 antibody. In some aspects, avelumab is administered as a flat dose of about 800 mg once about every 2 weeks.

II.B.3.b. CTLA-4 Inhibitors

In some aspects, the checkpoint inhibitor a disclosed herein comprises a CTLA-4 inhibitor. In some aspects, the CTLA-4 inhibitor is an anti-CTLA-4 antibody.

Anti-CTLA-4 antibodies that can be used in the methods of the disclosure bind to human CTLA-4 and disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances, or prolongs the activation of such T cells, thereby inducing, enhancing or prolonging an immune response.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 107 M⁻¹, or about 109 M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

Anti-CTLA-4 antibodies that can be used in the methods of the disclosure include ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)).

In some aspects, the anti-CTLA-4 antibody binds specifically to human CTLA-4 and cross-competes for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab.

In some aspects, the antibodies that cross-compete for binding to human CTLA-4 with, or bind to the same epitope region as, any anti-CTLA-4 antibody disclosed herein, e.g., ipilimumab and/or tremelimumab, are monoclonal antibodies. For administration to human subjects, these cross-competing antibodies are chimeric antibodies, engineered antibodies, or humanized or human antibodies.

Anti-CTLA-4 antibodies that can be used in the methods of the disclosure also include antigen-binding portions of any of the above full-length antibodies.

In some aspects, the anti-CTLA-4 antibody is a full-length antibody. In some aspects, the anti-CTLA-4 antibody is a monoclonal, human, humanized, chimeric, or multispecific antibody. In some aspects, the multispecific antibody is a DART, a DVD-Ig, or bispecific antibody.

In some aspects, the anti-CTLA-4 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-CTLA-4 antibody is ipilimumab, tremelimumab, MK-1308, AGEN-1884, or comprises an antigen binding portion thereof.

In some aspects, the anti-CTLA-4 antibody is ipilimumab. Ipilimumab is a fully human, IgG1 monoclonal antibody that blocks the binding of CTLA-4 to its B7 ligands, thereby stimulating T cell activation. In some aspects, ipilimumab is administered at a dose of about 3 mg/kg once about every 3 weeks. In some aspects, ipilimumab is administered at a dose of about 10 mg/kg once about every 3 weeks. In some aspects, ipilimumab is administered at a dose of about 10 mg/kg once about every 12 weeks. In some aspects, the ipilimumab is administered for four doses. In some aspects, ipilimumab is administered on Day 1 of each cycle.

### II.B.4. Therapies for Sensitizing Mutations

In some aspects, a method of the disclosure comprises treatment of a subject with a mutation sensitive to targeted inhibitor therapy such as a sensitizing mutation in a gene such as EGFR, ALK, ROS-1, NTRK, or BRAF. Such methods can further comprise administration of a targeted inhibitor of the mutated genes, including standard of care therapies for subjects having such mutations who are afflicted with NSCLC.

In some aspects, a method of the disclosure comprises a first line therapy for a subject afflicted with advanced or metastatic NSCLC who has a sensitizing EGFR mutation, comprising administering to the subject afatinib (e.g., 40 mg orally once daily), erlotinib (e.g., 150 mg orally once daily), dacomitinib (e.g., 45 mg orally once daily), gefitinib (e.g., 250 mg orally once daily), or osimertinib (e.g., 80 mg orally once daily).

In some aspects, a method of the disclosure comprises a second or third line therapy for a subject afflicted with advanced or metastatic NSCLC who has a sensitizing EGFR mutation, comprising administering to the subject afatinib and cetuximab (e.g., 40 mg afatinib orally once daily on Days 1-14 and cetuximab at 500 mg/m² on Day 1 in 2-week cycles) or osimertinib (e.g., 80 mg orally once daily).

In some aspects, a method of the disclosure comprises a first, second, or third line therapy for a subject afflicted with advanced or metastatic NSCLC who has a sensitizing ALK mutation (e.g., ALK rearrangement), comprising administering to the subject alectinib (e.g., 600 mg orally twice daily), brigatinib (e.g., a 4 week cycle of 90 mg orally once daily on Days 1-7, 180 mg orally once daily on days 8-28 followed by 180 mg orally once daily on Days 29-56), ceritinib (e.g., 450 mg orally once daily), or crizotinib (e.g., 250 mg orally twice daily).

In some aspects, a method of the disclosure comprises a second or third line therapy for a subject afflicted with advanced or metastatic NSCLC who has a sensitizing ALK mutation (e.g., ALK rearrangement), comprising administering to the subject lorlatinib (e.g., 100 mg orally once daily).

In some aspects, a method of the disclosure comprises a first line therapy for a subject afflicted with advanced or metastatic NSCLC who has a sensitizing ROS-1 mutation (e.g., ROS-1 rearrangement), comprising administering to the subject ceritinib (e.g., 450 mg orally once daily), crizotinib (e.g., 250 mg orally twice daily), or entrectinib (e.g., 600 mg orally once daily). In some aspects, a method of the disclosure comprises a standard of care second or third line therapy for a subject afflicted with advanced or metastatic NSCLC who has a sensitizing ROS-1 mutation (e.g., ROS-1 rearrangement), wherein the standard of care therapy comprises administering to the subject lorlatinib (e.g., 100 mg orally once daily).

In some aspects, a method of the disclosure comprises a first, second, or third line therapy for a subject afflicted with advanced or metastatic NSCLC who has a sensitizing BRAF mutation (e.g., BRAF V600E), comprising administering to the subject dabrafenib (e.g., 150 mg orally twice daily), dabrafenib and trametinib (e.g., 150 mg orally twice daily and 2 mg orally once daily trametinib), or vemurafenib (e.g., 960 mg orally once daily).

In some aspects, a method of the disclosure comprises a first, second, or third line therapy for a subject afflicted with advanced or metastatic NSCLC who has a sensitizing NTRK mutation (e.g., NTRK gene fusion), comprising administering to the subject entrectinib (e.g., 600 mg orally once daily) or larotrectinib (e.g., 100 mg orally twice daily).

### III. Pharmaceutical Compositions

Therapeutic agents of the present disclosure can be constituted in a composition, e.g., a pharmaceutical composition containing an inhibitor, antibody, and/or agent as disclosed herein and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible.

In some aspects, the carrier for a composition containing an inhibitor, antibody, and/or agent as disclosed herein is suitable for intravenous, intramuscular, subcutaneous, parenteral, spinal or epidermal administration (e.g., by injection or infusion). In some aspects, the carrier is suitable for non-parenteral, e.g., oral, administration. In some aspects, a subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an antibody with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. In some aspects, the pharmaceutical composition for the present disclosure can further comprise recombinant human hyaluronidase enzyme, e.g., rHuPH20.

Treatment is continued as long as clinical benefit is observed or until unacceptable toxicity or disease progression occurs. Dosage and frequency vary depending on the half-life of the inhibitor, antibody, and/or agent in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients (i.e., inhibitors, antibodies, and/or agents) in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

Provided herein is a pharmaceutical composition comprising an anti-LAG-3 antibody and an anti-PD-1 antibody as described herein at any of the doses or combinations of doses described herein.

In some aspects, the pharmaceutical composition is for treating a human subject with lung cancer as described herein.

In some aspects, a method for treating a human subject with lung cancer as described herein comprises administering a pharmaceutical composition as described herein.

In some aspects, the pharmaceutical composition comprises a dose of relatlimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is nivolumab.

In some aspects, the pharmaceutical composition comprises a dose of favezelimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is pembrolizumab.

In some aspects, the pharmaceutical composition comprises a dose of fianlimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is cemiplimab.

In some aspects, the pharmaceutical composition comprises a dose of ieramilimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is spartalizumab.

In some aspects, the pharmaceutical composition comprises a ratio of anti-LAG-3 antibody to anti-PD-1 antibody of about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1.

In some aspects, the pharmaceutical composition comprises a ratio of anti-LAG-3 antibody to anti-PD-1 antibody of about 1:3.

In some aspects, the pharmaceutical composition comprises a ratio of anti-LAG-3 antibody to anti-PD-1 antibody of about 1:1

In some aspects, the pharmaceutical composition comprises a ratio of anti-LAG-3 antibody to anti-PD-1 antibody of about 2:1.

In some aspects, the pharmaceutical composition comprises a ratio of anti-LAG-3 antibody to anti-PD-1 antibody of about 4:1.

In some aspects, the total amount of anti-LAG-3 and anti-PD-1 antibodies in the pharmaceutical composition is about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 205 mg/mL, about 210 mg/mL, about 215 mg/mL, about 220 mg/mL, about 225 mg/mL, about 230 mg/mL, about 235 mg/mL, about 240 mg/mL, about 245 mg/mL, about 250 mg/mL, about 255 mg/mL, about 260 mg/mL, about 265 mg/mL, about 270 mg/mL, about 275 mg/mL, about 280 mg/mL, about 285 mg/mL, about 290 mg/mL, about 295 mg/mL, about 300 mg/mL, about 305 mg/mL, about 310 mg/mL, about 315 mg/mL, about 320 mg/mL, about 325 mg/mL, about 330 mg/mL, about 335 mg/mL, about 340 mg/mL, about 345 mg/mL, about 350 mg/mL, about 355 mg/mL, about 360 mg/mL, about 365 mg/mL, about 370 mg/mL, about 375 mg/mL, about 380 mg/mL, about 385 mg/mL, about 390 mg/mL, about 395 mg/mL, about 400 mg/mL, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1580 mg, about 1590 mg, about 1600 mg, about 1610 mg, about 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, or about 1780 mg.

In some aspects, the total amount of anti-LAG-3 and anti-PD-1 antibodies in the pharmaceutical composition is about 25 mg/mL.

In some aspects, the total amount of anti-LAG-3 and anti-PD-1 antibodies in the pharmaceutical composition is about 50 mg/mL.

In some aspects, the total amount of anti-LAG-3 and anti-PD-1 antibodies in the pharmaceutical composition is about 150 mg/mL.

In some aspects, the total amount of anti-LAG-3 and anti-PD-1 antibodies in the pharmaceutical composition is about 50 mg.

In some aspects, the total amount of anti-LAG-3 and anti-PD-1 antibodies in the pharmaceutical composition is about 320 mg.

In some aspects, the total amount of anti-LAG-3 and anti-PD-1 antibodies in the pharmaceutical composition is about 640 mg.

In some aspects, the total amount of anti-LAG-3 and anti-PD-1 antibodies in the pharmaceutical composition is about 720 mg.

In some aspects, the total amount of anti-LAG-3 and anti-PD-1 antibodies in the pharmaceutical composition is about 960 mg.

In some aspects, the total amount of anti-LAG-3 and anti-PD-1 antibodies in the pharmaceutical composition is about 1000 mg.

In some aspects, the total amount of anti-LAG-3 and anti-PD-1 antibodies in the pharmaceutical composition is about 1080 mg.

In some aspects, the total amount of anti-LAG-3 and anti-PD-1 antibodies in the pharmaceutical composition is about 1440 mg.

In some aspects, the pharmaceutical composition comprises about 10 mg/mL, about 12.5 mg/mL, about 15 mg/mL, about 17.5 mg/mL, about 20 mg/mL, about 22.5 mg/mL, about 25 mg/mL, about 27.5 mg/mL, about 30 mg/mL, about 32.5 mg/mL, about 35 mg/mL, about 37.5 mg/mL, about 40 mg/mL, about 42.5 mg/mL, about 45 mg/mL, about 47.5 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 7 mg, about 21 mg, about 40 mg, about 70 mg, about 80 mg, about 160 mg, about 200 mg, about 210 mg, about 300 mg, about 400 mg, about 480 mg, about 500 mg, about 600 mg, about 700 mg, about 800 mg, about 900 mg, about 960 mg, about 1000 mg, about 1100 mg, about 1200 mg, or about 1300 mg of an anti-LAG-3 antibody. In some aspects, the pharmaceutical composition comprises about 5 mg/mL, about 10 mg/mL, about 12.5 mg/mL, about 15 mg/mL, about 17.5 mg/mL, about 20 mg/mL, about 22.5 mg/mL, about 25 mg/mL, about 27.5 mg/ml, about 30 mg/mL, about 32.5 mg/mL, about 35 mg/mL, about 37.5 mg/mL, about 40 mg/mL, about 42.5 mg/mL, about 45 mg/mL, about 47.5 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 10 mg, about 40 mg, about 100 mg, about 200 mg, about 240 mg, about 300 mg, about 350 mg, about 360 mg, about 400 mg, or about 480 mg of an anti-PD-1 antibody.

In some aspects, the pharmaceutical composition comprises about 12.5 mg/mL of an anti-LAG-3 antibody and about 37.5 mg/mL of an anti-PD-1 antibody.

In some aspects, the pharmaceutical composition comprises about 20 mg/mL of an anti-LAG-3 antibody and about 5 mg/mL of an anti-PD-1 antibody.

In some aspects, the pharmaceutical composition comprises about 75 mg/mL of an anti-LAG-3 antibody and about 75 mg/mL of an anti-PD-1 antibody.

In some aspects, the pharmaceutical composition comprises about 100 mg/mL of an anti-LAG-3 antibody and about 50 mg/mL of an anti-PD-1 antibody.

In some aspects, the pharmaceutical composition comprises about 80 mg of an anti-LAG-3 antibody and about 240 mg of an anti-PD-1 antibody.

In some aspects, the pharmaceutical composition comprises about 160 mg of an anti-LAG-3 antibody and about 480 mg of an anti-PD-1 antibody.

In some aspects, the pharmaceutical composition comprises about 360 mg of an anti-LAG-3 antibody and about 360 mg of an anti-PD-1 antibody.

In some aspects, the pharmaceutical composition comprises about 480 mg of an anti-LAG-3 antibody and about 480 mg of an anti-PD-1 antibody.

In some aspects, the pharmaceutical composition comprises about 720 mg of an anti-LAG-3 antibody and about 360 mg of an anti-PD-1 antibody.

In some aspects, the pharmaceutical composition comprises about 800 mg of an anti-LAG-3 antibody and about 200 mg of an anti-PD-1 antibody.

In some aspects, the pharmaceutical composition comprises about 960 mg of an anti-LAG-3 antibody and about 480 mg of an anti-PD-1 antibody.

In some aspects, the pharmaceutical composition comprises from about 5 mM to about 50 mM of histidine, from about 50 mM to about 300 mM of sucrose, from about 5 μM to about 1 mM of diethylenetriaminepentaacetic acid (DTPA) or ethylenediaminetetraacetic acid (EDTA), and from about 0.001% to about 1% (w/v) of polysorbate or poloxamer (e.g., polysorbate 80 (PS80), polysorbate 20 (PS20), poloxamer 188 (PX188), or any combination thereof).

In some aspects, the pharmaceutical composition comprises about 20 mM histidine, about 250 mM sucrose, about 50 μM DTPA, and 0.05% PS80.

In some aspects, the pH of the pharmaceutical composition is from about 5 to about 6.5. In some aspects, the pH is about 5.3 to about 6.3. In some aspects, the pH is 5.8. In some aspects, the pH is 5.7.

Provided herein is a vial, syringe, or intravenous bag comprising a pharmaceutical composition as described herein. In some aspects, the disclosure includes an autoinjector comprising a pharmaceutical composition described herein.

In some aspects, a vial comprises a pharmaceutical composition as described herein, and the vial further comprises a stopper and a seal. In some aspects, the total volume in the vial is about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL.

### IV. Kits

Also within the scope of the present invention are kits for treating a human subject with lung cancer comprising any of the antibodies, therapeutic agents, and/or anti-cancer therapies described herein.

Kits typically include a label indicating the intended use of the contents of the kit and instructions for use. The term “label” includes any writing, or recorded material supplied on or with the kit, or which otherwise accompanies the kit.

Provided herein is a kit for treating a human subject afflicted with lung cancer, comprising: (a) a dose of an anti-LAG-3 antibody; (b) a dose of an anti-PD-1 antibody; and (c) instructions for using the anti-LAG-3 antibody and the anti-PD-1 antibody in a method for treating a human subject afflicted with lung cancer.

The anti-LAG-3 antibody and the anti-PD-1 antibodies can be provided at any of the doses or combinations of doses described herein.

In some aspects, the kit comprises a dose of relatlimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is nivolumab.

In some aspects, the kit comprises a dose of favezelimab and a dose of an anti-PD-1 antibody as described herein. In some aspects, the anti-PD-1 antibody is nivolumab, pembrolizumab, cemiplimab, or spartalizumab. In some aspects, the anti-PD-1 antibody is pembrolizumab.

In some aspects, the kit comprises fianlimab and an anti-PD-1 antibody as described herein. In some aspects, the anti-PD-1 antibody is nivolumab, pembrolizumab, cemiplimab, or spartalizumab. In some aspects, the anti-PD-1 antibody is cemiplimab.

In some aspects, the kit comprises ieramilimab and an anti-PD-1 antibody as described herein. In some aspects, the anti-PD-1 antibody is nivolumab, pembrolizumab, cemiplimab, or spartalizumab. In some aspects, the anti-PD-1 antibody is spartalizumab.

In some aspects, the kit comprises a ratio of anti-LAG-3 antibody to anti-PD-1 antibody of about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1.

In some aspects, the kit comprises a ratio of anti-LAG-3 antibody to anti-PD-1 antibody of about 1:3.

In some aspects, the kit comprises a ratio of anti-LAG-3 antibody to anti-PD-1 antibody of about 1:1

In some aspects, the kit comprises a ratio of anti-LAG-3 antibody to anti-PD-1 antibody of about 2:1.

In some aspects, the kit comprises a ratio of anti-LAG-3 antibody to anti-PD-1 antibody of about 4:1.

In some aspects, the total amount of anti-LAG-3 and anti-PD-1 antibodies in the kit is about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 205 mg/mL, about 210 mg/mL, about 215 mg/mL, about 220 mg/mL, about 225 mg/mL, about 230 mg/mL, about 235 mg/mL, about 240 mg/mL, about 245 mg/mL, about 250 mg/mL, about 255 mg/mL, about 260 mg/mL, about 265 mg/mL, about 270 mg/mL, about 275 mg/mL, about 280 mg/mL, about 285 mg/mL, about 290 mg/mL, about 295 mg/mL, about 300 mg/mL, about 305 mg/mL, about 310 mg/mL, about 315 mg/mL, about 320 mg/mL, about 325 mg/mL, about 330 mg/mL, about 335 mg/mL, about 340 mg/mL, about 345 mg/mL, about 350 mg/mL, about 355 mg/mL, about 360 mg/mL, about 365 mg/mL, about 370 mg/mL, about 375 mg/mL, about 380 mg/mL, about 385 mg/mL, about 390 mg/mL, about 395 mg/mL, about 400 mg/mL, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1580 mg, about 1590 mg, about 1600 mg, about 1610 mg, about 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, or about 1780 mg.

In some aspects, the total amount of anti-LAG-3 and anti-PD-1 antibodies in the kit is about 25 mg/mL.

In some aspects, the total amount of anti-LAG-3 and anti-PD-1 antibodies in the kit is about 50 mg/mL.

In some aspects, the total amount of anti-LAG-3 and anti-PD-1 antibodies in the kit is about 150 mg/mL.

In some aspects, the total amount of anti-LAG-3 and anti-PD-1 antibodies in the kit is about 50 mg.

In some aspects, the total amount of anti-LAG-3 and anti-PD-1 antibodies in the kit is about 320 mg.

In some aspects, the total amount of anti-LAG-3 and anti-PD-1 antibodies in the kit is about 640 mg.

In some aspects, the total amount of anti-LAG-3 and anti-PD-1 antibodies in the kit is about 720 mg.

In some aspects, the total amount of anti-LAG-3 and anti-PD-1 antibodies in the kit is about 960 mg.

In some aspects, the total amount of anti-LAG-3 and anti-PD-1 antibodies in the kit is about 1000 mg.

In some aspects, the total amount of anti-LAG-3 and anti-PD-1 antibodies in the kit is about 1080 mg.

In some aspects, the total amount of anti-LAG-3 and anti-PD-1 antibodies in the kit is about 1440 mg.

In some aspects, the kit comprises about 10 mg/mL, about 12.5 mg/mL, about 15 mg/mL, about 17.5 mg/mL, about 20 mg/mL, about 22.5 mg/mL, about 25 mg/mL, about 27.5 mg/mL, about 30 mg/mL, about 32.5 mg/mL, about 35 mg/mL, about 37.5 mg/mL, about 40 mg/mL, about 42.5 mg/mL, about 45 mg/mL, about 47.5 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 7 mg, about 21 mg, about 40 mg, about 70 mg, about 80 mg, about 160 mg, about 200 mg, about 210 mg, about 300 mg, about 400 mg, about 480 mg, about 500 mg, about 600 mg, about 700 mg, about 800 mg, about 900 mg, about 960 mg, about 1000 mg, about 1100 mg, about 1200 mg, or about 1300 mg of an anti-LAG-3 antibody. In some aspects, the kit comprises about 5 mg/mL, about 10 mg/mL, about 12.5 mg/mL, about 15 mg/mL, about 17.5 mg/mL, about 20 mg/mL, about 22.5 mg/mL, about 25 mg/mL, about 27.5 mg/ml, about 30 mg/mL, about 32.5 mg/mL, about 35 mg/mL, about 37.5 mg/mL, about 40 mg/mL, about 42.5 mg/mL, about 45 mg/mL, about 47.5 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 10 mg, about 40 mg, about 100 mg, about 200 mg, about 240 mg, about 300 mg, about 350 mg, about 360 mg, about 400 mg, or about 480 mg of an anti-PD-1 antibody.

In some aspects, the kit comprises about 12.5 mg/mL of an anti-LAG-3 antibody and about 37.5 mg/mL of an anti-PD-1 antibody.

In some aspects, the kit comprises about 20 mg/mL of an anti-LAG-3 antibody and about 5 mg/mL of an anti-PD-1 antibody.

In some aspects, the kit comprises about 75 mg/mL of an anti-LAG-3 antibody and about 75 mg/mL of an anti-PD-1 antibody.

In some aspects, the kit comprises about 100 mg/mL of an anti-LAG-3 antibody and about 50 mg/mL of an anti-PD-1 antibody.

In some aspects, the kit comprises about 80 mg of an anti-LAG-3 antibody and about 240 mg of an anti-PD-1 antibody.

In some aspects, the kit comprises about 160 mg of an anti-LAG-3 antibody and about 480 mg of an anti-PD-1 antibody.

In some aspects, the kit comprises about 360 mg of an anti-LAG-3 antibody and about 360 mg of an anti-PD-1 antibody.

In some aspects, the kit comprises about 480 mg of an anti-LAG-3 antibody and about 480 mg of an anti-PD-1 antibody.

In some aspects, the kit comprises about 720 mg of an anti-LAG-3 antibody and about 360 mg of an anti-PD-1 antibody.

In some aspects, the kit comprises about 800 mg of an anti-LAG-3 antibody and about 200 mg of an anti-PD-1 antibody.

In some aspects, the kit comprises about 960 mg of an anti-LAG-3 antibody and about 480 mg of an anti-PD-1 antibody.

Provided herein is a kit for treating a human subject afflicted with lung cancer, comprising: (a) 360 mg of an anti-LAG-3 antibody; (b) 360 mg of an anti-PD-1 antibody; and (c) instructions for using the anti-LAG-3 antibody and the anti-PD-1 antibody in a method for treating a human subject afflicted with lung cancer.

Provided herein is a kit for treating a human subject afflicted with lung cancer, comprising: (a) 720 mg of an anti-LAG-3 antibody; (b) 360 mg of an anti-PD-1 antibody; and (c) instructions for using the anti-LAG-3 antibody and the anti-PD-1 antibody in a method for treating a human subject afflicted with lung cancer.

Provided herein is a kit for treating a human subject afflicted with lung cancer, comprising: (a) an anti-LAG-3 antibody; (b) an anti-PD-1 antibody; and (c) instructions for preparing each of the antibodies in an amount of 360 mg and using the antibodies in a method for treating a human subject afflicted with lung cancer.

Provided herein is a kit for treating a human subject afflicted with lung cancer, comprising: (a) an anti-LAG-3 antibody; (b) an anti-PD-1 antibody; and (c) instructions for preparing the anti-LAG-3 and anti-PD-1 antibodies in an amount of 720 mg and 360 mg, respectively, and using the antibodies in a method for treating a human subject afflicted with lung cancer.

In some aspects, the anti-LAG-3 and anti-PD-1 antibodies are co-packaged in a single unit dosage form.

In some aspects, the anti-LAG-3 and anti-PD-1 antibodies are packaged as separate unit dosage forms.

In some aspects, 40 mg of the anti-LAG-3 antibody is provided in a unit dosage form.

In some aspects, 80 mg of the anti-LAG-3 antibody is provided in a unit dosage form.

In some aspects, 160 mg of the anti-LAG-3 antibody is provided in a unit dosage form.

In some aspects, 360 mg of the anti-LAG-3 antibody is provided in a unit dosage form.

In some aspects, 480 mg of the anti-LAG-3 antibody is provided in a unit dosage form.

In some aspects, 720 mg of the anti-LAG-3 antibody is provided in a unit dosage form.

In some aspects, 800 mg of the anti-LAG-3 antibody is provided in a unit dosage form.

In some aspects, 960 mg of the anti-LAG-3 antibody is provided in a unit dosage form.

In some aspects, 12.5 mg/mL of the anti-LAG-3 antibody is provided in a unit dosage form.

In some aspects, 20 mg/mL of the anti-LAG-3 antibody is provided in a unit dosage form.

In some aspects, 50 mg/mL of the anti-LAG-3 antibody is provided in a unit dosage form.

In some aspects, 75 mg/mL of the anti-LAG-3 antibody is provided in a unit dosage form.

In some aspects, 100 mg/mL of the anti-LAG-3 antibody is provided in a unit dosage form.

In some aspects, 130 mg/mL of the anti-LAG-3 antibody is provided in a unit dosage form.

In some aspects, 150 mg/mL of the anti-LAG-3 antibody is provided in a unit dosage form.

In some aspects, 175 mg/mL of the anti-LAG-3 antibody is provided in a unit dosage form.

In some aspects, 200 mg/mL of the anti-LAG-3 antibody is provided in a unit dosage form.

In some aspects, 10 mg of the anti-PD-1 antibody is provided in a unit dosage form.

In some aspects, 40 mg of the anti-PD-1 antibody is provided in a unit dosage form.

In some aspects, 100 mg of the anti-PD-1 antibody is provided in a unit dosage form.

In some aspects, 200 mg of the anti-PD-1 antibody is provided in a unit dosage form.

In some aspects, 240 mg of the anti-PD-1 antibody is provided in a unit dosage form.

In some aspects, 360 mg of the anti-PD-1 antibody is provided in a unit dosage form.

In some aspects, 480 mg of the anti-PD-1 antibody is provided in a unit dosage form.

In some aspects, 5 mg/mL of the anti-PD-1 antibody is provided in a unit dosage form.

In some aspects, 10 mg/mL of the anti-PD-1 antibody is provided in a unit dosage form.

In some aspects, 37.5 mg/mL of the anti-PD-1 antibody is provided in a unit dosage form.

In some aspects, 50 mg/mL of the anti-PD-1 antibody is provided in a unit dosage form.

In some aspects, 75 mg/mL of the anti-PD-1 antibody is provided in a unit dosage form.

In some aspects, 100 mg/mL of the anti-PD-1 antibody is provided in a unit dosage form.

In some aspects, 175 mg/mL of the anti-PD-1 antibody is provided in a unit dosage form.

In some aspects, 200 mg/mL of the anti-PD-1 antibody is provided in a unit dosage form.

In some aspects, the unit dosage form comprises from about 5 mM to about 50 mM of histidine, from about 50 mM to about 300 mM of sucrose, from about 5 μM to about 1 mM of diethylenetriaminepentaacetic acid (DTPA) or ethylenediaminetetraacetic acid (EDTA), and from about 0.001% to about 1% (w/v) of polysorbate or poloxamer (e.g., polysorbate 80 (PS80), polysorbate 20 (PS20), poloxamer 188 (PX188), or any combination thereof).

In some aspects, the unit dosage form comprises about 20 mM histidine, about 250 mM sucrose, about 50 μM DTPA, and 0.05% PS80.

In some aspects, the unit dosage form comprises a pH of from about 5 to about 6.5. In some aspects, the pH is about 5.3 to about 6.3. In some aspects, the pH is 5.8. In some aspects, the pH is 5.7.

In some aspects, the unit dosage form is a vial, syringe, or intravenous bag. In some aspects, the unit dosage form is an autoinjector. In some aspects, the unit dosage form is a vial comprising a stopper and a seal. In some aspects, the total volume in the vial is about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL

In some aspects, the kit provides instructions for administering the anti-LAG-3 antibody and/or the anti-PD-1 antibody intravenously for about 30 minutes.

In some aspects, the kit further comprises therapeutic agents for one or more PDCTs as disclosed herein. In some aspects, the therapeutic agents for one or more PDCTs are carboplatin and paclitaxel, carboplatin and albumin-bound paclitaxel, carboplatin and pemetrexed, and/or cisplatin and pemetrexed. In some aspects, the therapeutic agents are carboplatin, cisplatin, paclitaxel, albumin-bound paclitaxel, and pemetrexed.

All of the references cited above, as well as all references cited herein, are incorporated herein by reference in their entireties.

The following examples are offered by way of illustration and not by way of limitation.

### EXAMPLES

### Example 1

### Combination of Anti-LAG-3 and Anti-PD-1 Antibodies with Chemotherapy for Treatment of Lung Cancer

A multi-center, randomized trial (“Study A”) will evaluate the efficacy and safety of the combination of nivolumab plus relatlimab with chemotherapy versus combination of nivolumab with chemotherapy in adults with untreated Stage IV or recurrent non-small cell lung cancer (NSCLC). The study will be carried out in 2 parts: Part 1, a site-and-subject blind dose safety confirmation; and Part 2, a double-blind, randomized, controlled trial.

Another multi-center, randomized trial (“Study B”) will evaluate the efficacy and safety of the combination of nivolumab plus relatlimab with chemotherapy versus combination of pembrolizumab with chemotherapy in adults with untreated Stage IV or recurrent NSCLC.

Patient Inclusion/Exclusion Criteria

Patients will be male and female adults ≥18 years or local age of majority selected based on the following eligibility criteria: (1) histologically confirmed metastatic NSCLC of squamous (SQ) or non-squamous (NSQ) histology with Stage IV A/B (as defined by the 8th International Association for the Study of Lung Cancer Classification) or recurrent disease following multi-modal therapy for locally advanced disease; (2) measurable disease by computed tomography or magnetic resonance imaging per RECIST v1.1 criteria with radiographic tumor assessment performed within 28 days before randomization; (3) no prior systemic anti-cancer treatment given as primary therapy for advanced or metastatic disease; (4) ECOG PS of ≤1 at screening and confirmed prior to randomization; (5) a life expectancy of at least 3 months at the time of first dose; (6) a formalin-fixed paraffin-embedded tissue block containing enough tissue to cut 20 sections or a minimum of 20 unstained slides of tumor tissue from core biopsy, punch biopsy, excisional biopsy, or surgical specimen obtained during screening or prior to enrollment (within 3 months of enrollment if stored at 2-8° C. or within 2 months of enrollment if stored at ambient temperature and with no intervening systemic anti-cancer treatment between time of acquisition and enrollment); and (6) PD-L1 and LAG-3 immunohistochemistry (IHC) results during the screening period prior to randomization (LAG-3 expression on immune cells and PD-L1 expression on tumor cells will be measured using analytically validated assays).

Prior definitive chemoradiation for locally advanced disease is permitted as long as the last administration of chemotherapy or radiotherapy (whichever was given last) occurred at least 6 months prior to enrollment. Prior adjuvant or neoadjuvant chemotherapy for early-stage lung cancer is permitted if completed at least 6 months prior to initiating study treatment. Prior palliative radiotherapy to non-central nervous system (CNS) lesions must have been completed at least 2 weeks prior to treatment. Participants with symptomatic tumor lesions at baseline that may require palliative radiotherapy within 4 weeks of first treatment are strongly encouraged to receive palliative radiotherapy prior to treatment.

Key exclusion criteria will be: (1) women who are pregnant or breastfeeding; (2) participants with EGFR, ALK, or ROS-1 mutations that are sensitive to available targeted inhibitor therapy (all participants with NSQ histology must have been tested for EGFR, ALK or ROS-1 mutation status; participants with NSQ histology and unknown EGFR, ALK, or ROS-1 status are excluded); (3) participants with known BRAF V600E mutations that are sensitive to available targeted inhibitor therapy (participants with unknown or indeterminate BRAF mutation status are eligible); (4) participants with untreated central nervous system metastases; (5) participants with leptomeningeal metastases (carcinomatous meningitis); (6) concurrent malignancy requiring treatment or history of prior malignancy active within 2 years prior to enrollment (i.e., participants with a history of prior malignancy are eligible if treatment was completed at least 2 years before registration and the participant has no evidence of disease); (7) participants with an active, known, or suspected autoimmune disease; (8) prior treatment with an anti-PD-1, anti-PD-L1, anti-PD-L2, anti-LAG-3, or anti-CTLA-4 antibody, or any other antibody or drug specifically targeting T-cell co-stimulation or checkpoint pathways; and (9) participants with history of myocarditis.

### Study A Design

### Part 1—Dose Safety Confirmation

Up to approximately 120 eligible participants will be randomized 1:1 to experimental Arms A or B. The randomization will be stratified by histology (SQ vs NSQ NSCLC).

Arm A: Nivolumab 360 mg administered every three weeks (Q3W)+relatlimab 720 mg Q3W+4 cycles of histology-based PDCT.

Arm B: Nivolumab 360 mg Q3W+relatlimab 360 mg Q3W+4 cycles of histology-based PDCT.

Histology-based PDCT will be as follows:

NSQ: Carboplatin area under the concentration-time curve (AUC) 5 or 6 or cisplatin 75 mg/m²+pemetrexed 500 mg/m² (maintenance with pemetrexed permitted following completion of the cycles of PDCT).

SQ: Carboplatin AUC 6⁺ paclitaxel 200 mg/m² or nab-paclitaxel (i.e., albumin-bound paclitaxel) 100 mg/m².

Nivolumab plus relatlimab will be administered in a site-and-subject blinded manner, whereas PDCT will be administered as open label.

The safety and tolerability of the combination of nivolumab plus relatlimab 720 mg and PDCT will be evaluated and the safety profile confirmed. The relatlimab 360 mg Q3W dose in Arm B will be evaluated to generate additional safety data at this dose level.

After all treated participants have been followed-up for a minimum of 12 weeks, the Part 1 safety data will be evaluated. In addition, the proportion of treatment-related adverse events (TRAEs) leading to discontinuation within 12 weeks of the first dose will be monitored for each arm separately using Bayesian continuous monitoring.

### Part 2—Efficacy and Safety

Part 2 will be a double-blind, randomized, controlled trial that will further evaluate the efficacy and safety of the nivolumab and relatlimab plus chemotherapy combination versus nivolumab plus chemotherapy. Only after the safety of nivolumab plus relatlimab and PDCT has been confirmed in Part 1 of the study can enrollment begin in Part 2 of the study. At this time, participants that are in screening and found to be eligible will be randomized 1:1 into experimental Arm C or control Arm D. Enrollment will end when approximately 400 participants have been randomized. The stratification factors for randomization in Part 2 are PD-L1 level (≥1% vs <1% [including non-quantifiable (NQ)]), LAG-3 expression level (≥1% vs <1% [including NQ]), histology (SQ vs NSQ), and gender (male vs female).

Arm C: Nivolumab 360 mg Q3W+relatlimab 720 mg or 360 mg Q3W+4 cycles of histology based PDCT.

Arm D: Nivolumab 360 mg Q3W+placebo Q3W+4 cycles of histology-based PDCT.

Histology-based PDCT is as described for Part 1 of the study.

All participants will be treated until progression, presence of intolerable toxicities, withdrawal of consent, or study end, whichever comes first. Continuous safety evaluations and tumor assessments will guide the decision to treat a participant with additional cycles of study therapy if the participant has confirmed clinical benefit.

Participants will be allowed to continue study treatment until the first occurrence of any of the following situations: (1) progressive disease defined by RECIST v1.1 unless participants meet criteria for treatment beyond progression; (2) clinical deterioration suggesting that no further benefit from treatment is likely; (3) intolerability to therapy; or (4) participant meets criteria for discontinuation of study treatment.

### Immunotherapy Dosing

Participants will receive masked nivolumab and relatlimab, followed by chemotherapy on Day 1 of every 3-week cycle. In Arms A, B, and C, nivolumab will be co-administered with relatlimab in a single bag IV over 30 minutes. In order to maintain blinding, Arm D participants will receive nivolumab+placebo (dextrose 5% or normal saline solution 0.9) IV also over 30 minutes. At the time of completion of the 4 cycles of chemotherapy, participants who have not experienced disease progression will continue to receive immunotherapy Q3W starting on Day 1 of the following cycle. There will be no dose escalations or reductions of immunotherapy allowed.

### Chemotherapy Dosing

In all 4 study arms, 4 cycles of the histology-based PDCT option selected by the investigator will be administered on Day 1 Q3W. Participants with NSQ histology may also receive optional maintenance therapy with 500 mg/m² pemetrexed alone on Day 1 of each 3-week cycle until disease progression or unacceptable toxicity.

For PDCT with paclitaxel and carboplatin, participants will receive paclitaxel 200 mg/m² as a 180-minute IV infusion followed by carboplatin at a dose of AUC 6 as a 30-minute IV infusion on Day 1 of a 3-week cycle, or at doses per the local prescribing information. The infusion time can follow local institutional standards.

For PDCT with nab-paclitaxel and carboplatin, participants will receive nab-paclitaxel 100 mg/m² as a 30-minute IV infusion on Days 1, 8, and 15 of each 21-day cycle. Carboplatin at a dose of AUC 6 as a 30-minute IV infusion will be administered immediately after nab-paclitaxel on Day 1 of each 3-week cycle or at doses per the local prescribing information. The infusion time can follow local institutional standards.

For PDCT with pemetrexed and cisplatin, participants will receive pemetrexed at a dose of 500 mg/m² as a 10-minute IV infusion on Day 1 with cisplatin at a dose of 75 mg/m² infusion as per local standard practice on Day 1 of a 3-week treatment cycle for up to 4 cycles. Cisplatin will be administered to participants at least 30 minutes following the end of the pemetrexed infusion.

For PDCT with pemetrexed and carboplatin, participants will receive pemetrexed at a dose of 500 mg/m² as a 10-minute IV infusion on Day 1, followed by carboplatin at a dose of AUC 5 or 6 as a 30-minute IV infusion on Day 1 of a 3-week treatment cycle, for up to 4 cycles.

After Cycle 4 of chemotherapy, participants with NSQ histology who have stable disease or response are permitted to receive pemetrexed 500 mg/m² alone as maintenance therapy until disease progression or unacceptable toxicity.

### Study B Design

Study B will be a double-blind, randomized, controlled trial that will further evaluate the safety and efficacy of the nivolumab and relatlimab plus chemotherapy combination versus pembrolizumab plus chemotherapy. Up to approximately 670 eligible participants will be randomized 1:1 to experimental Arm A or control Arm B. The randomization will be stratified by histology (SQ vs NSQ NSCLC), PD-L1 level (≥1% vs <1% [including non-quantifiable (NQ)]), LAG-3 expression level (≥1% vs <1% [including NQ]), and gender (male vs female).

Arm A: Nivolumab 360 mg Q3W+relatlimab 720 mg+4 cycles of histology based PDCT.

Arm B: Pembrolizumab 200 mg Q3W+4 cycles of histology-based PDCT.

Histology-based PDCT is as described for Part 1 of Study A.

All participants will be treated until progression, presence of intolerable toxicities, withdrawal of consent, or for 2 years, whichever comes first. Scans will occur Q6W for 1 year, then Q12W until progression or discontinuation of the study.

Immunotherapy and chemotherapy dosing will be conducted in a manner similar to Study A.

### Example 2

### Clinical Activity of Anti-LAG-3 Antibody in Combination with Anti-PD-1 Antibody in Patients with Lung Cancer

Anti-LAG-3 antibody (relatlimab) in combination with anti-PD-1 antibody (nivolumab) was evaluated as a first line treatment of NSCLC.

A tumor tissue sample was obtained from each patient for determination of LAG-3 expression. Patients were stratified as LAG-3 expressers or non-expressers based on LAG-3 expression in tissue samples of ≥1% or less than 1%, respectively.

Patients were treated with 80 mg of relatlimab once every 2 weeks in combination with 240 mg nivolumab once every 2 weeks.

The best overall response (BOR) summary for all response evaluable subjects is shown in Table 1. The objective response rate (ORR) was defined as the proportion of treated subjects whose BOR was either a complete response (CR) or a partial response (PR) based on blinded independent clinical review (BICR) assessments by RECIST 1.1 Criteria. 2-sided 95% exact confidence intervals were determined by the Clopper-Pearson method.

TABLE 1
Best overall response summary

| | | LAG-3 | | |
|---|---|---|---|---|
| | LAG-3 | Non- | LAG-3 | All |
| | Expressers | Expressers | Evaluable | Subjects |
| Best Overall Response (BOR) (%) | N = 10 | N = 5 | N = 15 | N = 23 |
| Complete Response (CR) | 1 (10.0) | 0 | 1 (6.7) | 1 (4.3) |
| Partial Response (PR) | 5 (50.0) | 1 (20.0) | 6 (40.0) | 6 (26.1) |
| Stable Disease (SD) | 3 (30.0) | 1 (20.0) | 4 (26.7) | 6 (26.1) |
| Stable Disease (≥12 weeks) | 3 (30.0) | 1 (20.0) | 4 (26.7) | 5 (21.7) |
| Progressive Disease (PD) | 1 (10.0) | 1 (20.0) | 2 (13.3) | 7 (30.4) |
| Non-CR/Non-PD | 0 | 0 | 0 | 0 |
| Unable to Determine | 0 | 2 (40.0) | 2 (13.3) | 3 (13.0) |
| Confirmed ORR (A) (%) | 6 (60.0) | 1 (20.0) | 7 (46.7) | 7 (30.4) |
| 95% Confidence Limit | (26.2, 87.8) | (0.5, 71.6) | (21.3, 73.4) | (13.2, 52.9) |
| Confirmed CR + PR + SD (A) (%) | 9 (90.0) | 2 (40.0) | 11 (73.3) | 13 (56.5) |
| 95% Confidence Limit | (55.5, 99.7) | (5.3, 85.3) | (44.9, 92.2) | (34.5, 76.8) |
| Confirmed DCR (12 W) (A) (%) | 9 (90.0) | 2 (40.0) | 11 (73.3) | 12 (52.2) |
| 95% Confidence Limits | (55.5, 99.7) | (5.3, 85.3) | (44.9, 92.2) | (30.6, 73.2) |

(A) = Confirmed Response Only
DCR(12 W) = Disease Control Rate = CR + PR + SD at ≥12 weeks

SEQUENCES
Heavy Chain Amino Acid Sequence;
Anti-LAG-3 mAb (BMS-986016)
SEQ ID NO: 1
QVQLQQWGAGLLKPSETLSLTCAVYGGSFSDYYWNWIRQPPGKGL
EWIGEINHRGSTNSNPSLKSRVTLSLDTSKNQFSLKLRSVTAADT
AVYYCAFGYSDYEYNWFDPWGQGTLVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFLGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVFSCSVMHEALHNHYTQKSLSLSLGK
Light Chain Amino Acid Sequence;
Anti-LAG-3 mAb (BMS-986016)
SEQ ID NO: 2
EIVLTQSPATLSLSPGERATLSCRASQSISSYLAWYQQKPGQAPR
LLIYDASNRATGIPARFSGSGSGTDFTLTISSLEPEDFAVYYCQQ
RSNWPLTFGQGTNLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Heavy Chain Variable Region (VH)
Amino Acid Sequence;
Anti-LAG-3 mAb
(BMS-986016)
SEQ ID NO: 3
QVQLQQWGAGLLKPSETLSLTCAVYGGSFSDYYWNWIRQPPGKGL
EWIGEINHRGSTNSNPSLKSRVTLSLDTSKNQFSLKLRSVTAADT
AVYYCAFGYSDYEYNWFDPWGQGTLVTVSS
Light Chain Variable Region (VL)
Amino Acid Sequence;
Anti-LAG-3 mAb (BMS-986016)
SEQ ID NO: 4
EIVLTQSPATLSLSPGERATLSCRASQSISSYLAWYQQKPGQAPR
LLIYDASNRATGIPARFSGSGSGTDFTLTISSLEPEDFAVYYCQQ
RSNWPLTFGQGTNLEIK
Heavy Chain CDR1 Amino Acid Sequence;
Anti-LAG-3 mAb (BMS-986016)
SEQ ID NO: 5
DYYWN
Heavy Chain CDR2 Amino Acid Sequence;
Anti-LAG-3 mAb (BMS-986016)
SEQ ID NO: 6
EINHRGSTNSNPSLKS
Heavy Chain CDR3 Amino Acid Sequence;
Anti-LAG-3 mAb (BMS-986016)
SEQ ID NO: 7
GYSDYEYNWFDP
Light Chain CDR1 Amino Acid Sequence;
Anti-LAG-3 mAb (BMS-986016)
SEQ ID NO: 8
RASQSISSYLA
Light Chain CDR2 Amino Acid Sequence;
Anti-LAG-3 mAb (BMS-986016)
SEQ ID NO: 9
DASNRAT
Light Chain CDR3 Amino Acid Sequence;
Anti-LAG-3 mAb (BMS-986016)
SEQ ID NO: 10
QQRSNWPLT
Heavy Chain Amino Acid Sequence;
Anti-PD-1 mAb (BMS-936558)
SEQ ID NO: 11
QVQLVESGGGVVQPGRSLRLDCKASGITFSNSGMHWVRQAPGKGL
EWVAVIWYDGSKRYYADSVKGRFTISRDNSKNTLFLQMNSLRAED
TAVYYCATNDDYWGQGTLVTVSSASTKGPSVFPLAPCSRSTSEST
AALGCLVKDYFPEPVTVSWNSGALTSGVHTFPAVLQSSGLYSLSS
VVTVPSSSLGTKTYTCNVDHKPSNTKVDKRVESKYGPPCPPCPAP
EFLGGPSVFLFPPKPKDTLMISRTPEVTCVVVDVSQEDPEVQFNW
YVDGVEVHNAKTKPREEQFNSTYRVVSVLTVLHQDWLNGKEYKCK
VSNKGLPSSIEKTISKAKGQPREPQVYTLPPSQEEMTKNQVSLTC
LVKGFYPSDIAVEWESNGQPENNYKTTPPVLDSDGSFFLYSRLTV
DKSRWQEGNVFSCSVMHEALHNHYTQKSLSLSLGK
Light Chain Amino Acid Sequence;
Anti-PD-1 mAb (BMS-936558)
SEQ ID NO: 12
EIVLTQSPATLSLSPGERATLSCRASQSVSSYLAWYQQKPGQAPR
LLIYDASNRATGIPARFSGSGSGTDFTLTISSLEPEDFAVYYCQQ
SSNWPRTFGQGTKVEIKRTVAAPSVFIFPPSDEQLKSGTASVVCL
LNNFYPREAKVQWKVDNALQSGNSQESVTEQDSKDSTYSLSSTLL
SKADYEKHKVYACEVTHQGLSSPVTKSFNRGEC
Heavy Chain Variable Region (VH)
Amino Acid Sequence;
Anti-PD-1 mAb
(BMS-936558)
SEQ ID NO: 13
QVQLVESGGGVVQPGRSLRLDCKASGITFSNSGMHWVRQAPGKGL
EWVAVIWYDGSKRYYADSVKGRFTISRDNSKNTLFLQMNSLRAED
TAVYYCATNDDYWGQGTLVTVSS
Light Chain Variable Region (VL)
Amino Acid Sequence;
Anti-PD-1 mAb (BMS-936558)
SEQ ID NO: 14
EIVLTQSPATLSLSPGERATLSCRASQSVSSYLAWYQQKPGQAPR
LLIYDASNRATGIPARFSGSGSGTDFTLTISSLEPEDFAVYYCQQ
SSNWPRTFGQGTKVEIK
Heavy Chain CDR1 Amino Acid Sequence;
Anti-PD-1 mAb (BMS-936558)
SEQ ID NO: 15
NSGMH
Heavy Chain CDR2 Amino Acid Sequence;
Anti-PD-1 mAb (BMS-936558)
SEQ ID NO: 16
VIWYDGSKRYYADSVKG
Heavy Chain CDR3 Amino Acid Sequence;
Anti-PD-1 mAb (BMS-936558)
SEQ ID NO: 17
NDDY
Light Chain CDR1 Amino Acid Sequence;
Anti-PD-1 mAb (BMS-936558)
SEQ ID NO: 18
RASQSVSSYLA
Light Chain CDR2 Amino Acid Sequence;
Anti-PD-1 mAb (BMS-936558)
SEQ ID NO: 19
DASNRAT
Light Chain CDR3 Amino Acid Sequence;
Anti-PD-1 mAb (BMS-936558)
SEQ ID NO: 20
QQSSNWPRT
Heavy Chain Amino Acid Sequence;
Anti-LAG-3 mAb (BMS-986016) without
terminal lysine
SEQ ID NO: 21
QVQLQQWGAGLLKPSETLSLTCAVYGGSFSDYYWNWIRQPPGKGL
EWIGEINHRGSTNSNPSLKSRVTLSLDTSKNQFSLKLRSVTAADT
AVYYCAFGYSDYEYNWFDPWGQGTLVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFLGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVFSCSVMHEALHNHYTQKSLSLSLG
Lymphocyte Activation Gene 3 Protein
Amino Acid Sequence (Homo Sapiens,
NP_002277)
SEQ ID NO: 22
MWEAQFLGLLFLQPLWVAPVKPLQPGAEVPVVWAQEGAPAQLPCS
PTIPLQDLSLLRRAGVTWQHQPDSGPPAAAPGHPLAPGPHPAAPS
SWGPRPRRYTVLSVGPGGLRSGRLPLQPRVQLDERGRQRGDFSLW
LRPARRADAGEYRAAVHLRDRALSCRLRLRLGQASMTASPPGSLR
ASDWVILNCSFSRPDRPASVHWFRNRGQGRVPVRESPHHHLAESF
LFLPQVSPMDSGPWGCILTYRDGFNVSIMYNLTVLGLEPPTPLTV
YAGAGSRVGLPCRLPAGVGTRSFLTAKWTPPGGGPDLLVTGDNGD
FTLRLEDVSQAQAGTYTCHIHLQEQQLNATVTLAIITVTPKSFGS
PGSLGKLLCEVTPVSGQERFVWSSLDTPSQRSFSGPWLEAQEAQL
LSQPWQCQLYQGERLLGAAVYFTELSSPGAQRSGRAPGALPAGHL
LLFLILGVLSLLLLVTGAFGFHLWRRQWRPRRFSALEQGIHPPQA
QSKIEELEQEPEPEPEPEPEPEPEPEPEQL
Heavy Chain Amino Acid Sequence;
Anti-LAG-3 mAb (REGN3767)
SEQ ID NO: 23
QVQLVESGGGVVQPGRSLRLSCVASGFTFSSYGMHWVRQAPGKGL
EWVAIIWYDGSNKYYADSVKGRFTISRDNSKNTQYLQMNSLRAED
TAVYYCASVATSGDFDYYGMDVWGQGTTVTVSSASTKGPSVFPLA
PCSRSTSESTAALGCLVKDYFPEPVTVSWNSGALTSGVHTFPAVL
QSSGLYSLSSVVTVPSSSLGTKTYTCNVDHKPSNTKVDKRVESKY
GPPCPPCPAPPVAGPSVFLFPPKPKDTLMISRTPEVTCVVVDVSQ
EDPEVQFNWYVDGVEVHNAKTKPREEQFNSTYRVVSVLTVLHQDW
LNGKEYKCKVSNKGLPSSIEKTISKAKGQPREPQVYTLPPSQEEM
TKNQVSLTCLVKGFYPSDIAVEWESNGQPENNYKTTPPVLDSDGS
FFLYSRLTVDKSRWQEGNVFSCSVMHEALHNHYTQKSLSLSLGK
Light Chain Amino Acid Sequence;
Anti-LAG-3 mAb (REGN3767)
SEQ ID NO: 24
EIVLTQSPATLSLSPGERTTLSCRASQRISTYLAWYQQKPGQAPR
LLIYDASKRATGIPARFSGSGSGTGFTLTISSLEPEDFAVYYCQQ
RSNWPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC
Heavy Chain Variable Region (VH)
Amino Acid Sequence;
Anti-LAG-3 mAb
(REGN3767)
SEQ ID NO: 25
QVQLVESGGGVVQPGRSLRLSCVASGFTFSSYGMHWVRQAPGKGL
EWVAIIWYDGSNKYYADSVKGRFTISRDNSKNTQYLQMNSLRAED
TAVYYCASVATSGDFDYYGMDVWGQGTTVTVSS
Light Chain Variable Region (VL)
Amino Acid Sequence;
Anti-LAG-3 mAb
(REGN3767)
SEQ ID NO: 26
EIVLTQSPATLSLSPGERTTLSCRASQRISTYLAWYQQKPGQAPR
LLIYDASKRATGIPARFSGSGSGTGFTLTISSLEPEDFAVYYCQQ
RSNWPLTFGGGTKVEIK
Heavy Chain CDR1 Amino Acid Sequence;
Anti-LAG-3 mAb (REGN3767)
SEQ ID NO: 27
GFTFSSYG
Heavy Chain CDR2 Amino Acid Sequence;
Anti-LAG-3 mAb (REGN3767)
SEQ ID NO: 28
IWYDGSNK
Heavy Chain CDR3 Amino Acid Sequence;
Anti-LAG-3 mAb (REGN3767)
SEQ ID NO: 29
ASVATSGDFDYYGMDV
Light Chain CDR1 Amino Acid Sequence;
Anti-LAG-3 mAb (REGN3767)
SEQ ID NO: 30
QRISTY
Light Chain CDR2 Amino Acid Sequence;
Anti-LAG-3 mAb (REGN3767)
SEQ ID NO: 31
DAS
Light Chain CDR3 Amino Acid Sequence;
Anti-LAG-3 mAb (REGN3767)
SEQ ID NO: 32
QQRSNWPLT
Heavy Chain Amino Acid Sequence;
Anti-PD-1 mAb (REGN2810)
SEQ ID NO: 33
EVQLLESGGVLVQPGGSLRLSCAASGFTFSNFGMTWVRQAPGKGL
EWVSGISGGGRDTYFADSVKGRFTISRDNSKNTLYLQMNSLKGED
TAVYYCVKWGNIYFDYWGQGTLVTVSSASTKGPSVFPLAPCSRST
SESTAALGCLVKDYFPEPVTVSWNSGALTSGVHTFPAVLQSSGLY
SLSSVVTVPSSSLGTKTYTCNVDHKPSNTKVDKRVESKYGPPCPP
CPAPEFLGGPSVFLFPPKPKDTLMISRTPEVTCVVVDVSQEDPEV
QFNWYVDGVEVHNAKTKPREEQFNSTYRVVSVLTVLHQDWLNGKE
YKCKVSNKGLPSSIEKTISKAKGQPREPQVYTLPPSQEEMTKNQV
SLTCLVKGFYPSDIAVEWESNGQPENNYKTTPPVLDSDGSFFLYS
RLTVDKSRWQEGNVFSCSVMHEALHNHYTQKSLSLSLGK
Light Chain Amino Acid Sequence;
Anti-PD-1 mAb (REGN2810)
SEQ ID NO: 34
DIQMTQSPSSLSASVGDSITITCRASLSINTFLNWYQQKPGKAPN
LLIYAASSLHGGVPSRFSGSGSGTDFTLTIRTLQPEDFATYYCQQ
SSNTPFTFGPGTVVDFRRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Heavy Chain Variable Region (VH)
Amino Acid Sequence;
Anti-PD-1 mAb
(REGN2810)
SEQ ID NO: 35
EVQLLESGGVLVQPGGSLRLSCAASGFTFSNFGMTWVRQAPGKGL
EWVSGISGGGRDTYFADSVKGRFTISRDNSKNTLYLQMNSLKGED
TAVYYCVKWGNIYFDYWGQGTLVTVSS
Light Chain Variable Region (VL)
Amino Acid Sequence;
Anti-PD-1 mAb
(REGN2810)
SEQ ID NO: 36
DIQMTQSPSSLSASVGDSITITCRASLSINTFLNWYQQKPGKAPN
LLIYAASSLHGGVPSRFSGSGSGTDFTLTIRTLQPEDFATYYCQQ
SSNTPFTFGPGTVVDFR
Heavy Chain CDR1 Amino Acid Sequence;
Anti-PD-1 mAb (REGN2810)
GFTFSNFG
SEQ ID NO: 37
Heavy Chain CDR2 Amino Acid Sequence;
Anti-PD-1 mAb (REGN2810)
SEQ ID NO: 38
ISGGGRDT
Heavy Chain CDR3 Amino Acid Sequence;
Anti-PD-1 mAb (REGN2810)
SEQ ID NO: 39
VKWGNIYFDY
Light Chain CDR1 Amino Acid Sequence;
Anti-PD-1 mAb (REGN2810)
SEQ ID NO: 40
LSINTF
Light Chain CDR2 Amino Acid Sequence;
Anti-PD-1 mAb (REGN2810)
SEQ ID NO: 41
AAS
SEQ ID NO: 42
Light Chain CDR3 Amino Acid Sequence;
Anti-PD-1 mAb (REGN2810)
QQSSNTPFT
Heavy Chain Amino Acid Sequence;
Anti-LAG-3 mAb (LAG525)
SEQ ID NO: 43
QVQLVQSGAEVKKPGASVKVSCKASGFTLTNYGMNWVRQARGQRL
EWIGWINTDTGEPTYADDFKGRFVFSLDTSVSTAYLQISSLKAED
TAVYYCARNPPYYYGTNNAEAMDYWGQGTTVTVSSASTKGPSVFP
LAPCSRSTSESTAALGCLVKDYFPEPVTVSWNSGALTSGVHTFPA
VLQSSGLYSLSSVVTVPSSSLGTKTYTCNVDHKPSNTKVDKRVES
KYGPPCPPCPAPEFLGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQ
EEMTKNQVSLTCLVKGFYPSDIAVEWESNGQPENNYKTTPPVLDS
DGSFFLYSRLTVDKSRWQEGNVFSCSVMHEALHNHYTQKSLSLSL
G
Heavy Chain Amino Acid Sequence;
Anti-LAG-3 mAb (LAG525)
SEQ ID NO: 44
QVQLVQSGAEVKKPGASVKVSCKASGFTLTNYGMNWVRQAPGQGL
EWMGWINTDTGEPTYADDFKGRFVFSLDTSVSTAYLQISSLKAED
TAVYYCARNPPYYYGTNNAEAMDYWGQGTTVTVSSASTKGPSVFP
LAPCSRSTSESTAALGCLVKDYFPEPVTVSWNSGALTSGVHTFPA
VLQSSGLYSLSSVVTVPSSSLGTKTYTCNVDHKPSNTKVDKRVES
KYGPPCPPCPAPEFLGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQ
EEMTKNQVSLTCLVKGFYPSDIAVEWESNGQPENNYKTTPPVLDS
DGSFFLYSRLTVDKSRWQEGNVFSCSVMHEALHNHYTQKSLSLSL
G
Light Chain Amino Acid Sequence;
Anti-LAG-3 mAb (LAG525)
SEQ ID NO: 45
DIQMTQSPSSLSASVGDRVTITCSSSQDISNYLNWYLQKPGQSPQ
LLIYYTSTLHLGVPSRFSGSGSGTEFTLTISSLQPDDFATYYCQQ
YYNLPWTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Light Chain Amino Acid Sequence;
Anti-LAG-3 mAb (LAG525)
SEQ ID NO: 46
DIQMTQSPSSLSASVGDRVTITCSSSQDISNYLNWYQQKPGKAPK
LLIYYTSTLHLGIPPRFSGSGYGTDFTLTINNIESEDAAYYFCQQ
YYNLPWTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Heavy Chain Variable Region (VH)
Amino Acid Sequence;
Anti-LAG-3 mAb
(LAG525)
SEQ ID NO: 47
QVQLVQSGAEVKKPGASVKVSCKASGFTLTNYGMNWVRQARGQRL
EWIGWINTDTGEPTYADDFKGRFVFSLDTSVSTAYLQISSLKAED
TAVYYCARNPPYYYGTNNAEAMDYWGQGTTVTVSS
Heavy Chain Variable Region (VH)
Amino Acid Sequence;
Anti-LAG-3 mAb
(LAG525)
SEQ ID NO: 48
QVQLVQSGAEVKKPGASVKVSCKASGFTLTNYGMNWVRQAPGQGL
EWMGWINTDTGEPTYADDFKGRFVFSLDTSVSTAYLQISSLKAED
TAVYYCARNPPYYYGTNNAEAMDYWGQGTTVTVSS
Light Chain Variable Region (VL)
Amino Acid Sequence;
Anti-LAG-3 mAb
(LAG525)
SEQ ID NO: 49
DIQMTQSPSSLSASVGDRVTITCSSSQDISNYLNWYLQKPGQSPQ
LLIYYTSTLHLGVPSRFSGSGSGTEFTLTISSLQPDDFATYYCQQ
YYNLPWTFGQGTKVEIK
Light Chain Variable Region (VL)
Amino Acid Sequence;
Anti-LAG-3 mAb
(LAG525)
SEQ ID NO: 50
DIQMTQSPSSLSASVGDRVTITCSSSQDISNYLNWYQQKPGKAPK
LLIYYTSTLHLGIPPRFSGSGYGTDFTLTINNIESEDAAYYFCQQ
YYNLPWTFGQGTKVEIK
Heavy Chain CDR1 Amino Acid Sequence;
Anti-LAG-3 mAb (LAG525)
SEQ ID NO: 51
NYGMN
Heavy Chain CDR2 Amino Acid Sequence;
Anti-LAG-3 mAb (LAG525)
SEQ ID NO: 52
WINTDTGEPTYADDFKG
Heavy Chain CDR3 Amino Acid Sequence;
Anti-LAG-3 mAb (LAG525)
SEQ ID NO: 53
NPPYYYGTNNAEAMDY
Light Chain CDR1 Amino Acid Sequence;
Anti-LAG-3 mAb (LAG525)
SEQ ID NO: 54
SSSQDISNYLN
Light Chain CDR2 Amino Acid Sequence;
Anti-LAG-3 mAb (LAG525)
SEQ ID NO: 55
YTSTLHL
Light Chain CDR3 Amino Acid Sequence;
Anti-LAG-3 mAb (LAG525)
SEQ ID NO: 56
QQYYNLPWT
Heavy Chain Amino Acid Sequence;
Anti-PD-1 mAb (PDR001)
SEQ ID NO: 57
EVQLVQSGAEVKKPGESLRISCKGSGYTFTTYWMHWVRQATGQGL
EWMGNIYPGTGGSNFDEKFKNRVTITADKSTSTAYMELSSLRSED
TAVYYCTRWTTGTGAYWGQGTTVTVSSASTKGPSVFPLAPCSRST
SESTAALGCLVKDYFPEPVTVSWNSGALTSGVHTFPAVLQSSGLY
SLSSVVTVPSSSLGTKTYTCNVDHKPSNTKVDKRVESKYGPPCPP
CPAPEFLGGPSVFLFPPKPKDTLMISRTPEVTCVVVDVSQEDPEV
QFNWYVDGVEVHNAKTKPREEQFNSTYRVVSVLTVLHQDWLNGKE
YKCKVSNKGLPSSIEKTISKAKGQPREPQVYTLPPSQEEMTKNQV
SLTCLVKGFYPSDIAVEWESNGQPENNYKTTPPVLDSDGSFFLYS
RLTVDKSRWQEGNVFSCSVMHEALHNHYTQKSLSLSLG
Light Chain Amino Acid Sequence;
Anti-PD-1 mAb (PDR001)
SEQ ID NO: 58
EIVLTQSPATLSLSPGERATLSCKSSQSLLDSGNQKNFLTWYQQK
PGQAPRLLIYWASTRESGVPSRFSGSGSGTDFTFTISSLEAEDAA
TYYCQNDYSYPYTFGQGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC
Heavy Chain Variable Region (VH)
Amino Acid Sequence;
Anti-PD-1 mAb
(PDR001)
SEQ ID NO: 59
EVQLVQSGAEVKKPGESLRISCKGSGYTFTTYWMHWVRQATGQGL
EWMGNIYPGTGGSNFDEKFKNRVTITADKSTSTAYMELSSLRSED
TAVYYCTRWTTGTGAYWGQGTTVTVSS
Light Chain Variable Region (VL)
Amino Acid Sequence;
Anti-PD-1 mAb
(PDR001)
SEQ ID NO: 60
EIVLTQSPATLSLSPGERATLSCKSSQSLLDSGNQKNFLTWYQQK
PGQAPRLLIYWASTRESGVPSRFSGSGSGTDFTFTISSLEAEDAA
TYYCQNDYSYPYTFGQGTKVEIK
Heavy Chain CDR1 Amino Acid Sequence;
Anti-PD-1 mAb (PDR001)
SEQ ID NO: 61
TYWMH
Heavy Chain CDR2 Amino Acid Sequence;
Anti-PD-1 mAb (PDR001)
SEQ ID NO: 62
NIYPGTGGSNFDEKFKN
Heavy Chain CDR3 Amino Acid Sequence;
Anti-PD-1 mAb (PDR001)
SEQ ID NO: 63
WTTGTGAY
Light Chain CDR1 Amino Acid Sequence;
Anti-PD-1 mAb (PDR001)
SEQ ID NO: 64
KSSQSLLDSGNQKNFLT
Light Chain CDR2 Amino Acid Sequence;
Anti-PD-1 mAb (PDR001)
SEQ ID NO: 65
WASTRES
Light Chain CDR3 Amino Acid Sequence;
Anti-PD-1 mAb (PDR001)
SEQ ID NO: 66
QNDYSYPYT
Heavy Chain Amino Acid Sequence;
Anti-LAG-3 mAb (MK4280)
SEQ ID NO: 67
QMQLVQSGPEVKKPGTSVKVSCKASGYTFTDYNVDWVRQARGQRL
EWIGDINPNDGGTIYAQKFQERVTITVDKSTSTAYMELSSLRSED
TAVYYCARNYRWFGAMDHWGQGTTVTVSSASTKGPSVFPLAPCSR
STSESTAALGCLVKDYFPEPVTVSWNSGALTSGVHTFPAVLQSSG
LYSLSSVVTVPSSSLGTKTYTCNVDHKPSNTKVDKRVESKYGPPC
PPCPAPEFLGGPSVFLFPPKPKDTLMISRTPEVTCVVVDVSQEDP
EVQFNWYVDGVEVHNAKTKPREEQFNSTYRVVSVLTVLHQDWLNG
KEYKCKVSNKGLPSSIEKTISKAKGQPREPQVYTLPPSQEEMTKN
QVSLTCLVKGFYPSDIAVEWESNGQPENNYKTTPPVLDSDGSFFL
YSRLTVDKSRWQEGNVFSCSVMHEALHNHYTQKSLSLSLGK
Light Chain Amino Acid Sequence;
Anti-LAG-3 mAb (MK4280)
SEQ ID NO: 68
DIVMTQTPLSLSVTPGQPASISCKASQSLDYEGDSDMNWYLQKPG
QPPQLLIYGASNLESGVPDRFSGSGSGTDFTLKISRVEAEDVGVY
YCQQSTEDPRTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Heavy Chain Variable Region (VH)
Amino Acid Sequence;
Anti-LAG-3 mAb
(MK4280)
SEQ ID NO: 69
QMQLVQSGPEVKKPGTSVKVSCKASGYTFTDYNVDWVRQARGQRL
EWIGDINPNDGGTIYAQKFQERVTITVDKSTSTAYMELSSLRSED
TAVYYCARNYRWFGAMDHWGQGTTVTVSS
Light Chain Variable Region (VL)
Amino Acid Sequence;
Anti-LAG-3 Anti-LAG-3 mAb (MK4280)
SEQ ID NO: 70
DIVMTQTPLSLSVTPGQPASISCKASQSLDYEGDSDMNWYLQKPG
QPPQLLIYGASNLESGVPDRFSGSGSGTDFTLKISRVEAEDVGVY
YCQQSTEDPRTFGGGTKVEIK
Heavy Chain CDR1 Amino Acid Sequence;
Anti-LAG-3 mAb (MK4280)
SEQ ID NO: 71
DYNVD
Heavy Chain CDR2 Amino Acid Sequence;
Anti-LAG-3 mAb (MK4280)
SEQ ID NO: 72
DINPNDGGTIYAQKFQE
Heavy Chain CDR3 Amino Acid Sequence;
Anti-LAG-3 mAb (MK4280)
SEQ ID NO: 73
NYRWFGAMDH
Light Chain CDR1 Amino Acid Sequence;
Anti-LAG-3 mAb (MK4280)
SEQ ID NO: 74
KASQSLDYEGDSDMN
Light Chain CDR2 Amino Acid Sequence;
Anti-LAG-3 mAb (MK4280)
SEQ ID NO: 75
GASNLES
Light Chain CDR3 Amino Acid Sequence;
Anti-LAG-3 mAb (MK4280)
SEQ ID NO: 76
QQSTEDPRT
Heavy Chain Amino Acid Sequence;
Anti-PD-1 mAb (MK3475)
SEQ ID NO: 77
QVQLVQSGVEVKKPGASVKVSCKASGYTFTNYYMYWVRQAPGQGL
EWMGGINPSNGGTNFNEKFKNRVTLTTDSSTTTAYMELKSLQFDD
TAVYYCARRDYRFDMGFDYWGQGTTVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFLGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVFSCSVMHEALHNHYTQKSLSLSLGK
Light Chain Amino Acid Sequence;
Anti-PD-1 mAb (MK3475)
SEQ ID NO: 78
EIVLTQSPATLSLSPGERATLSCRASKGVSTSGYSYLHWYQQKPG
QAPRLLIYLASYLESGVPARFSGSGSGTDFTLTISSLEPEDFAVY
YCQHSRDLPLTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Heavy Chain Variable Region (VH) Amino
Acid Sequence;
Anti-PD-1 mAb
(MK3475)
SEQ ID NO: 79
QVQLVQSGVEVKKPGASVKVSCKASGYTFTNYYMYWVRQAPGQGL
EWMGGINPSNGGTNFNEKFKNRVTLTTDSSTTTAYMELKSLQFDD
TAVYYCARRDYRFDMGFDYWGQGTTVTVSS
Light Chain Variable Region (VL) Amino Acid
Sequence;
Anti-PD-1 mAb (MK3475)
SEQ ID NO: 80
EIVLTQSPATLSLSPGERATLSCRASKGVSTSGYSYLHWYQQKPG
QAPRLLIYLASYLESGVPARFSGSGSGTDFTLTISSLEPEDFAVY
YCQHSRDLPLTFGGGTKVEIK
Heavy Chain CDR1 Amino Acid Sequence;
Anti-PD-1 mAb (MK3475)
SEQ ID NO: 81
NYYMY
Heavy Chain CDR2 Amino Acid Sequence;
Anti-PD-1 mAb (MK3475)
SEQ ID NO: 82
GINPSNGGTNFNEKFKN
Heavy Chain CDR3 Amino Acid Sequence;
Anti-PD-1 mAb (MK3475)
SEQ ID NO: 83
RDYRFDMGFDY
Light Chain CDR1 Amino Acid Sequence;
Anti-PD-1 mAb (MK3475)
SEQ ID NO: 84
RASKGVSTSGYSYLH
Light Chain CDR2 Amino Acid Sequence;
Anti-PD-1 mAb (MK3475)
SEQ ID NO: 85
LASYLES
Light Chain CDR3 Amino Acid Sequence;
Anti-PD-1 mAb (MK3475)
SEQ ID NO: 86
QHSRDLPLT

1-220. (canceled)

221. A method of treating a human subject afflicted with a lung cancer, the method comprising administering to the subject a lymphocyte activation gene-3 (LAG-3) antagonist and a platinum doublet chemotherapy (PDCT).

222. The method of claim 221, wherein the LAG-3 antagonist comprises an anti-LAG-3 antibody and/or a soluble LAG-3 polypeptide.

223. The method of claim 222, wherein the anti-LAG-3 antibody is:
(a) a full-length antibody,
(b) a monoclonal, human, humanized, chimeric, or multispecific antibody, or
(c) a F(ab′)₂ fragment, a Fab′ fragment, a Fab fragment, a Fv fragment, a scFv fragment, a dsFv fragment, a dAb fragment, or a single chain binding polypeptide.

224. The method of claim 222, wherein the anti-LAG-3 antibody comprises:
(a) BMS-986016 (relatlimab), IMP731 (H5L7BW), MK-4280 (28G-10, favezelimab), REGN3767 (fianlimab), GSK2831781, humanized BAP050, IMP-701 (LAG-525, ieramilimab), aLAG3(0414), aLAG3(0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGNO2385, IBI-110, EMB-02, IBI-323, LBL-007, ABL501, or an antigen binding portion thereof,
(b) CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4,
(c) a heavy chain variable region CDR1, CDR2, and CDR3 comprising the sequences set forth in SEQ ID NOs:5, 6, and 7, respectively, and a light chain variable region CDR1, CDR2, and CDR3 comprising the sequences set forth in SEQ ID NOs:8, 9, and 10, respectively,
(d) heavy and light chain variable regions comprising the sequences set forth in SEQ ID NOs:3 and 4, respectively,
(e) heavy and light chains comprising the sequences set forth in SEQ ID NOs:1 and 2, respectively, or
(f) heavy and light chains comprising the sequences set forth in SEQ ID NOs:21 and 2, respectively.

225. The method of claim 221, further comprising administering to the subject a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

226. The method of claim 225, wherein the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

227. The method of claim 226, wherein the PD-1 pathway inhibitor comprises an anti-PD-1 antibody, an anti-PD-L1 antibody, a soluble PD-L2 polypeptide, and/or BMS-986189.

228. The method of claim 227, wherein the anti-PD-1 antibody and/or the anti-PD-L1 antibody is:
(a) a full-length antibody,
(b) a monoclonal, human, humanized, chimeric, or multispecific antibody,
(c) a F(ab′)₂ fragment, a Fab′ fragment, a Fab fragment, a Fv fragment, a scFv fragment, a dsFv fragment, a dAb fragment, or a single chain binding polypeptide.

229. The method of claim 227, wherein the anti-PD-1 antibody comprises:
(a) nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or an antigen binding portion thereof,
(b) CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14,
(c) a heavy chain variable region CDR1, CDR2, and CDR3 comprising the sequences set forth in SEQ ID NOs:15, 16, and 17, respectively, and a light chain variable region CDR1, CDR2, and CDR3 comprising the sequences set forth in SEQ ID NOs:18, 19, and 20, respectively,
(d) heavy and light chain variable regions comprising the sequences set forth in SEQ ID NOs:13 and 14, respectively, or
(e) heavy and light chains comprising the sequences set forth in SEQ ID NOs:11 and 12, respectively.

230. The method of claim 221, wherein the lung cancer is a small cell lung cancer or a non-small cell lung cancer (NSCLC).

231. The method of claim 221, wherein the PDCT comprises a platinum agent in combination with a nucleoside analog, an antimetabolite, a taxane, a vinca alkaloid, or a topisomerase inhibitor.

232. The method of claim 221, wherein one or more immune cells in tumor tissue from the subject express LAG-3 and/or one or more tumor cells in tumor tissue from the subject express PD-L1.

233. The method of claim 232, wherein:
(a) at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3, and/or
(b) at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1.

234. A method of treating a human subject afflicted with a lung cancer, the method comprising administering to the subject:
(a) a dose of about 360 mg or 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:4, and
(b) a dose of about 360 mg of an anti-PD-1 antibody comprising CDR1, CDR2 and CDR3 domains of the heavy chain variable region having the sequence set forth in SEQ ID NO:13, and CDR1, CDR2 and CDR3 domains of the light chain variable region having the sequence set forth in SEQ ID NO:14.

235. The method of claim 234, wherein the lung cancer is a small cell lung cancer or a non-small cell lung cancer (NSCLC).

236. The method of claim 234, further comprising administering a PDCT.

237. The method of claim 236, wherein the PDCT comprises a platinum agent in combination with a nucleoside analog, an antimetabolite, a taxane, a vinca alkaloid, or a topisomerase inhibitor.

238. The method of claim 234, wherein the subject is afflicted with Stage IV or recurrent NSCLC that has a squamous histology, the PDCT comprises:
(i) a dose of carboplatin for a target area under the concentration-time curve of about 6 mg/mL·min, and
(ii) a dose of about 200 mg/m² of paclitaxel or about 100 mg/m² of albumin-bound paclitaxel, and
the method is a first line therapy.

239. The method of claim 234, wherein the subject is afflicted with Stage IV or recurrent NSCLC that has a non-squamous histology, the PDCT comprises:
(i) a dose of carboplatin for a target area under the concentration-time curve of about 5 mg/mL·min or about 6 mg/mL·min or about 75 mg/m² of cisplatin, and
(ii) a dose of about 500 mg/m² of pemetrexed, and
the method is a first line therapy.

240. A pharmaceutical composition for treating a lung cancer comprising: (a) 360 mg or 720 mg of an anti-LAG-3 antibody, and (b) 360 mg of an anti-PD-1 antibody.

241. The pharmaceutical composition of claim 240, wherein:
(a) the anti-LAG-3 antibody comprises CDR1, CDR2, and CDR3 domains of the heavy chain variable region having the sequence set forth in SEQ ID NO:3, and CDR1, CDR2, and CDR3 domains of the light chain variable region having the sequence set forth in SEQ ID NO:4, and the anti-PD-1 antibody comprises CDR1, CDR2, and CDR3 domains of the heavy chain variable region having the sequence set forth in SEQ ID NO:13, and CDR1, CDR2, and CDR3 domains of the light chain variable region having the sequence set forth in SEQ ID NO:14,
(b) the anti-LAG-3 antibody comprises a heavy chain variable region CDR1, CDR2, and CDR3 comprising the sequence set forth in SEQ ID NO:5, SEQ ID NO:6, and SEQ ID NO:7, respectively, and a light chain variable region CDR1, CDR2, and CDR3 comprising the sequence set forth in SEQ ID NO:8, SEQ ID NO:9, and SEQ ID NO:10, respectively, and the anti-PD-1 antibody comprises a heavy chain variable region CDR1, CDR2, and CDR3 comprising the sequence set forth in SEQ ID NO:15, SEQ ID NO:16, and SEQ ID NO:17, respectively, and a light chain variable region CDR1, CDR2, and CDR3 comprising the sequence set forth in SEQ ID NO:18, SEQ ID NO:19, and SEQ ID NO:20, respectively,
(c) the anti-LAG-3 antibody comprises heavy and light chain variable regions comprising the sequences set forth in SEQ ID NOs:3 and 4, respectively, and the anti-PD-1 antibody comprises heavy and light chain variable regions comprising the sequences set forth in SEQ ID NOs:13 and 14, respectively,
(d) the anti-LAG-3 antibody comprises heavy and light chains comprising the sequences set forth in SEQ ID NOs:1 and 2, respectively, and the anti-PD-1 antibody comprises heavy and light chains comprising the sequences as set forth in SEQ ID NOs:11 and 12, respectively, or
(e) the anti-LAG-3 antibody comprises heavy and light chains comprising the sequences set forth in SEQ ID NOs:21 and 2, respectively, and the anti-PD-1 antibody comprises heavy and light chains comprising the sequences as set forth in SEQ ID NOs:11 and 12, respectively.

242. A kit for treating a human subject afflicted with a lung cancer comprising: the pharmaceutical composition of claim 240, and instructions for using the anti-LAG-3 antibody and the anti-PD-1 antibody in a method for treating a human subject afflicted with lung cancer.
